(19) United States
(12) Patent Application Publication
(10) Pub. No.: US 2025/0082840 A1
(43) Pub. Date: Mar. 13, 2025

(54) GLUCOSE LEVEL CONTROL SYSTEM SMART CHARGING STATION

(71) Applicant: Beta Bionics, Inc., Concord, MA (US)

(72) Inventors: Himanshu Patel, Concord, MA (US); Michael J. Rosinko, Concord, MA (US); Firas H. Khatib, Concord, MA (US)

(73) Assignee: Beta Bionics, Inc., Concord, MA (US)

(21) Appl. No.: 18/725,245

(22) PCT Filed: Dec. 27, 2022
(86) PCT No.: PCT/US2022/054099

§ 371 Date: Jun. 28, 2024

Related U.S. Application Data
(60) Provisional application No. 63/295,361, filed on Dec. 30, 2021.

(51) Int. Cl. A61M 5/142; A61M 5/172; G16H 20/17; G16H 40/67

(52) U.S. Cl. CPC A61M 5/14244; A61M 5/1723; G16H 20/17; G16H 40/67; A61M 2205/18; A61M 2205/3569; A61M 2205/3592; A61M 2205/8243; A61M 2230/201

(57) ABSTRACT

A smart charging station may be capable of accessing data from an ambulatory medicament device at particular times, such as when the ambulatory medicament device is charging or connected to the charging station. The smart charging station may then communicate the data to another electronic system, such as a remote system. Advantageously, the charging station enables an ambulatory medicament device that may not be capable of communicating with a remote system to transfer ambulatory medicament device data to the remote system. Further, the remote system may communicate with the ambulatory medicament device via the charging station enabling, for example, control parameter updates, software updates, or permission modifications, among other communications with the ambulatory medicament device regardless of whether direct communication with the ambulatory medicament device is possible.

### INCORPORATION BY REFERENCE TO ANY PRIORITY APPLICATIONS

This application claims priority to U.S. Provisional Application No. 63/295,361, filed on Dec. 30, 2021, which is hereby incorporated by reference in its entirety for all purposes herein and made a part of the present specification. Any and all applications for which a foreign or domestic priority claim is identified in the Application Data Sheet as filed with the present application are hereby incorporated by reference under 37 CFR 1.57.

### TECHNICAL FIELD

The present disclosure relates to ambulatory medical devices, such as blood glucose control systems, that provide therapy to a subject.

### BACKGROUND

Sustained delivery, pump driven medicament injection devices generally include a delivery cannula mounted in a subcutaneous manner through the skin of the patient at an infusion site. The pump draws medicine from a reservoir and delivers it to the patient via the cannula. The injection device typically includes a channel that transmits a medicament from an inlet port to the delivery cannula which results in delivery to the subcutaneous tissue layer where the delivery cannula terminates. Some infusion devices are configured to deliver one medicament to a patient while others are configured to deliver multiple medicaments to a patient.

Some pump driven medicament devices use a closed loop control system to control the amount of medicament supplied to a subject. The closed loop control system may determine the amount of medicament to supply based on one or more sensor readings obtained from one or more sensors configured to measure one or more physiological characteristics of the subject.

### SUMMARY

The systems, methods, and devices of this disclosure each have several innovative aspects, no single one of which is solely responsible for all the desirable attributes disclosed herein. Details of one or more implementations of the subject matter described in this specification are set forth in the accompanying drawings and the description below.

In some aspects, the techniques described herein relate to a charging station configured to receive ambulatory medicament device data from an ambulatory medicament device, the charging station including: a charging assembly configured to charge a battery of the ambulatory medicament device; a memory configured to at least store specific computer-executable instructions; a first transceiver configured to at least receive the ambulatory medicament device data from the ambulatory medicament device; a second transceiver configured to at least transmit the ambulatory medicament device data received from the ambulatory medicament device; and a hardware processor in communication with the memory and configured to execute the specific computer-executable instructions to at least: detect a charge connection to the ambulatory medicament device; initiate charging of the ambulatory medicament device using the charging assembly; determine a destination identifier for the ambulatory medicament device data; receive, via the first transceiver, the ambulatory medicament device data from the ambulatory medicament device; and transmit, using the second transceiver, the ambulatory medicament device data to a remote system corresponding to the destination identifier.

In some aspects, the techniques described herein relate to a computer-implemented method of receiving ambulatory medicament device data from an ambulatory medicament device by a charging station including a charging assembly configured to charge a battery of the ambulatory medicament device; a first transceiver configured to at least receive the ambulatory medicament device data from the ambulatory medicament device; and a second transceiver configured to at least transmit the ambulatory medicament device data received from the ambulatory medicament device, the computer-implemented method including: by a hardware processor of the charging station, detecting a charge connection to the ambulatory medicament device; initiating charging of the ambulatory medicament device using the charging assembly; determining a destination identifier for the ambulatory medicament device data; receiving, via the first transceiver, the ambulatory medicament device data from the ambulatory medicament device; and transmitting, using the second transceiver, the ambulatory medicament device data to a remote system corresponding to the destination identifier.

In some aspects, the techniques described herein relate to an ambulatory medicament device configured to transmit ambulatory medicament device data to a charging station, the ambulatory medicament device including: a battery configured to power the ambulatory medicament device; a charging circuit configured to receive power from the charging station and to charge the battery; a memory configured to at least store the ambulatory medicament device data and specific computer-executable instructions; a transceiver configured to at least transmit the ambulatory medicament device data to the charging station; and a hardware processor in communication with the memory and configured to execute the specific computer-executable instructions to at least: detect a charge connection to the charging station; initiate, via the charging circuit, charging of the battery over the charge connection; determine whether the charging station supports a data connection to the ambulatory medicament device; and in response to determining that the charging station supports the data connection: establish the data connection to the charging station; automatically select, without user interaction with a user interface, ambulatory medicament device data using one or more of a plurality of data selection criteria; prepare the ambulatory medicament device data for transmission to the charging station via the data connection; and automatically transmit, without user interaction with the user interface, the ambulatory medicament device data to the charging station enabling communication of the ambulatory medicament device data by the charging station to a remote system separate from the charging station without the ambulatory medicament device establishing communication with the remote system.

In some aspects, the techniques described herein relate to a computer-implemented method of transmitting ambulatory medicament device data to a charging station by an ambulatory medicament device including a charging circuit configured to receive power from the charging station and to charge a battery of the ambulatory medicament device, and a transceiver configured to at least transmit the ambulatory medicament device data to the charging station, the computer-implemented method including: by a hardware processor of the ambulatory medicament device, detecting a charge connection to the charging station; initiating, via the charging circuit, charging of the battery over the charge connection; determining whether the charging station supports a data connection to the ambulatory medicament device; and in response to determining that the charging station supports the data connection: establishing the data connection to the charging station; automatically selecting, without user interaction with a user interface, ambulatory medicament device data using one or more of a plurality of data selection criteria; preparing the ambulatory medicament device data for transmission to the charging station via the data connection; and automatically transmitting, without user interaction with the user interface, the ambulatory medicament device data to the charging station enabling communication of the ambulatory medicament device data by the charging station to a remote system separate from the charging station without the ambulatory medicament device establishing communication with the remote system.

In some aspects, the techniques described herein relate to an ambulatory medicament device configured to modify or enable modification of a control algorithm of the ambulatory medicament device based on configuration data accessed from a configuration chip, the ambulatory medicament device including: a chip reader configured to read data stored on the configuration chip; a memory configured to store specific computer-executable instructions; and a hardware processor in communication with the memory and configured to execute the specific computer-executable instructions to at least: cause the chip reader to emit an electromagnetic pulse; in response to the electromagnetic pulse, receive the configuration data from the configuration chip; decode the configuration data to obtain a value corresponding to a control parameter, wherein the control parameter is used by the control algorithm that generates a dose control signal to cause the ambulatory medicament device to administer a quantity of medicament from a medicament reservoir into a subject; and configure the control parameter based at least in part on the value decoded from the configuration data.

In some aspects, the techniques described herein relate to a computer-implemented method of modifying or enabling modification of a control algorithm of an ambulatory medicament device based on configuration data accessed from a configuration chip using a chip reader of the ambulatory medicament device, the computer-implemented method including: by a hardware processor of the ambulatory medicament device, causing the chip reader to emit an electromagnetic pulse; in response to the electromagnetic pulse, receiving the configuration data from the configuration chip; decoding the configuration data to obtain a value corresponding to a control parameter, wherein the control parameter is used by the control algorithm that generates a dose control signal to cause the ambulatory medicament device to administer a quantity of medicament from a medicament reservoir into a subject; and configuring the control parameter based at least in part on the value decoded from the configuration data.

### BRIEF DESCRIPTION OF THE DRAWINGS

Throughout the drawings, reference numbers are re-used to indicate correspondence between referenced elements. The drawings are provided to illustrate certain aspects of the subject matter described herein and not to limit the scope thereof.

FIG. 1A illustrates a first example of an ambulatory medicament device environment for controlling a glucose level of a subject via an ambulatory medicament pump.

FIG. 1B illustrates a second example of an ambulatory medicament device environment for controlling a glucose level of a subject via an ambulatory medicament pump.

FIG. 1C illustrates a third example of an ambulatory medicament device environment for controlling a glucose level of a subject via an ambulatory medicament pump.

FIG. 2A illustrates a block diagram of a first example of a glucose level control system.

FIG. 2B illustrates a block diagram of a second example of a glucose level control system.

FIG. 2C illustrates a block diagram of a third example of a glucose level control system.

FIG. 2D illustrates a block diagram of a fourth example of a glucose level control system.

FIG. 3 illustrates a block diagram of an example glucose level control system that includes an electronic communications interface.

FIG. 4A illustrates a block diagram of an example of elements of a glucose level control system operating in an online mode.

FIG. 4B illustrates a block diagram of an example of elements of a glucose level control system operating in an offline mode.

FIG. 5A illustrates a block diagram of an example glucose level control system environment that includes a glucose level control system in accordance with certain embodiments.

FIG. 5B illustrates a block diagram of a second example glucose level control system environment in accordance with certain embodiments.

FIG. 5C illustrates a block diagram of a third example glucose level control system environment in accordance with certain embodiments.

FIG. 6 illustrates a block diagram of an example controller in accordance with certain embodiments.

FIG. 7A illustrates an example of a charging station configured to charge and/or communicate with an ambulatory medicament device in accordance with certain embodiments.

FIG. 7B illustrates an example of an ambulatory medicament device being positioned on the charging station of FIG. 7A in accordance with certain embodiments.

FIG. 7C illustrates an example of the ambulatory medicament device being charged by the charging station of FIG. 7A in accordance with certain embodiments.

FIG. 7D illustrates an example of data communication between the ambulatory medicament device and the charging station of FIG. 7A in accordance with certain embodiments.

FIG. 8 illustrates a block diagram of an ambulatory medicament device interacting with a configuration chip and/or a charging station in accordance with certain embodiments.

FIG. 9A illustrates a flowchart of an example charging station based ambulatory medicament device data transfer process in accordance with certain embodiments.

FIG. 9B illustrates a flowchart of an example remote server based data transfer process in accordance with certain embodiments.

FIG. 10 illustrates a flowchart of an example automatic ambulatory medicament device data selection and transfer process in accordance with certain embodiments.

FIG. 11 illustrates a flowchart of an example ambulatory medicament device configuration process in accordance with certain embodiments.

### DETAILED DESCRIPTION

Some embodiments described herein pertain to medicament infusion systems for one or more medicaments and the components of such systems (e.g., infusion pumps, medicament cartridges, cartridge connectors, lumen assemblies, infusion connectors, infusion sets, etc.). Some embodiments pertain to methods of manufacturing infusion systems and components thereof. Some embodiments pertain to methods of using any of the foregoing systems or components for infusing one or more medicaments (e.g., pharmaceutical, hormone, etc.) to a patient. As an exemplary illustration, an infusion system may include an infusion pump, which can include one or more medicament cartridges or can have an integrated reservoir of medicament. An infusion system may include medicament cartridges and cartridge connectors, but not a pump. An infusion system may include cartridge connectors and an infusion pump, but not medicament cartridges. An infusion system may include infusion connectors, a lumen assembly, cartridge connectors, an infusion pump, but not medicament cartridges or an infusion set A blood glucose control system can operate in conjunction with an infusion system to infuse one or more medicaments, including at least one blood glucose control agent, into a subject. Any feature, structure, component, material, step, or method that is described and/or illustrated in any embodiment in this specification can be used with or instead of any feature, structure, component, material, step, or method that is described and/or illustrated in any other embodiment in this specification. Additionally, any feature, structure, component, material, step, or method that is described and/or illustrated in one embodiment may be absent from another embodiment.

Some patients manage their diabetes by injecting insulin, which may be referred to as injection therapy. Other patients use medicament pumps to help manage their diabetes. These medicament pumps may be controlled manually or may be closed-loop systems that autonomously provide medicament therapy. For example, a glucose level control system may operate in a closed loop mode that enables the glucose level control system to automatically determine insulin dosing using a control algorithm and one or more sensor signals received at a sensor interface from one or more sensors. These sensors may include continuous glucose monitoring (CGM) sensors operatively coupled to a subject. The CGM sensors may provide measurements of blood glucose levels of the subject to the blood glucose control system, which may autonomously determine the insulin dose using the measurements.

Glucose level control systems that autonomously determine a quantity of medicament (e.g., insulin or counter-regulatory agent, such as Glucagon) to supply to a patient are becoming more common. The use of glucose level control systems and medicament pumps free the patient from the inconvenience of injection therapy. Further, the automated closed-loop control algorithms are generally more accurate and provide better disease maintenance than other diabetes management options. For example, using test strips to measure glucose levels and performing injection therapy based on the test strips tends to be less accurate as it may not account for previously injected insulin or recent food consumption.

In some cases, the glucose level control system may collect data (which for ease of description and not to limit the type of data may be referred to as “ambulatory medicament device data” herein) associated with operation of the glucose level control system. This ambulatory medicament device data may relate to therapy provided to the subject, operating status of the glucose level control system, user-interaction with the glucose level control system, automated or manual modifications to a control algorithm that controls the medicament therapy and/or operation of the glucose level control system, and/or any other type of information that may be generated or collected by the glucose level control system. It can be useful for maintaining the subject's disease, monitoring the status of the glucose level control system, learning how to treat other people who have the same disease as the subject, generating backup therapy protocols, or for any number of additional reasons to communicate the ambulatory medicament device data to another electronic system, such as an electronic system of the subject, a healthcare provider, a disease researcher, a glucose level control system manufacturer, or any other number of authorized systems associated with authorized users. In some cases, the glucose level control system is part of an ambulatory medicament device that includes the capability of directly communicating with the electronic system regardless of whether the electronic system is local (e.g., within the same physical space or network space as the ambulatory medicament device) or remote (e.g., on a different network than the ambulatory medicament device or accessible via a wide-area network). However, in other cases, the ambulatory medicament device may not be capable of communicating directly with a remote electronic system.

Regardless of whether the glucose level control system is closed-loop, open-loop, or partially closed-loop, it is often necessary to charge a battery used to power the glucose level control system. In some cases, the battery may be charged via charging station.

Embodiments disclosed herein relate to a smart charging station, or a charging station capable of accessing data from the ambulatory medicament device at particular times, such as when the ambulatory medicament device is charging or connected to the charging station, and communicating the data to another electronic system, such as a remote system. Advantageously, the charging station enables an ambulatory medicament device that may not be capable of communicating with a remote system to transfer ambulatory medicament device data to the remote system. Further, in some embodiments, the remote system may communicate with the ambulatory medicament device via the charging station enabling, for example, control parameter updates, software updates, or permission modifications, among other communications.

The ambulatory medicament device may communicate data with the charging station using a communication system that implements a short-range or near-field communication protocol. In some cases, the communication system may be capable of communication with a configuration chip. The configuration chip may communicate configuration data, such as a configuration code, that may be used to configure the ambulatory medicament device. In some cases, the configuration data may be used to perform an initial configuration of the ambulatory medicament device. Alternatively, or in addition, the configuration data may be used to update an existing configuration of the ambulatory medicament device. The configuration data may be used to configure a replacement ambulatory medicament device, to implement control algorithm updates to the ambulatory medicament system, or to otherwise configure or reconfigure the ambulatory medicament system.

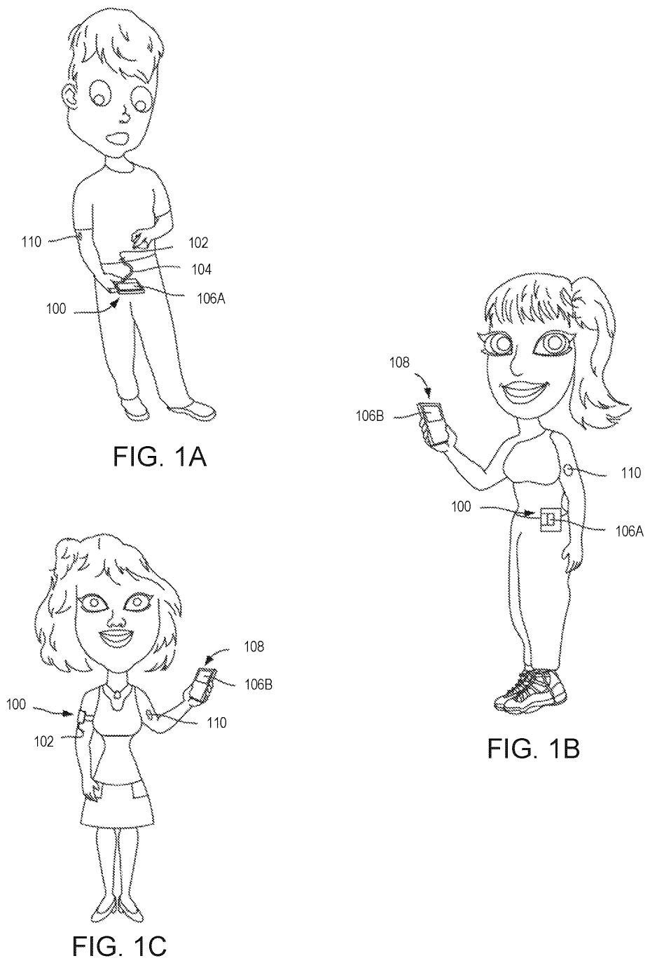

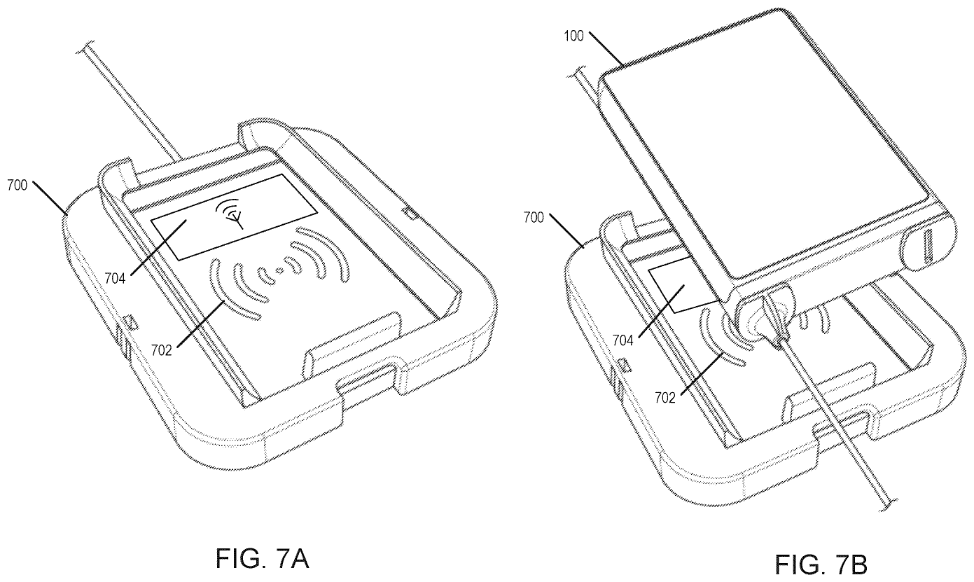

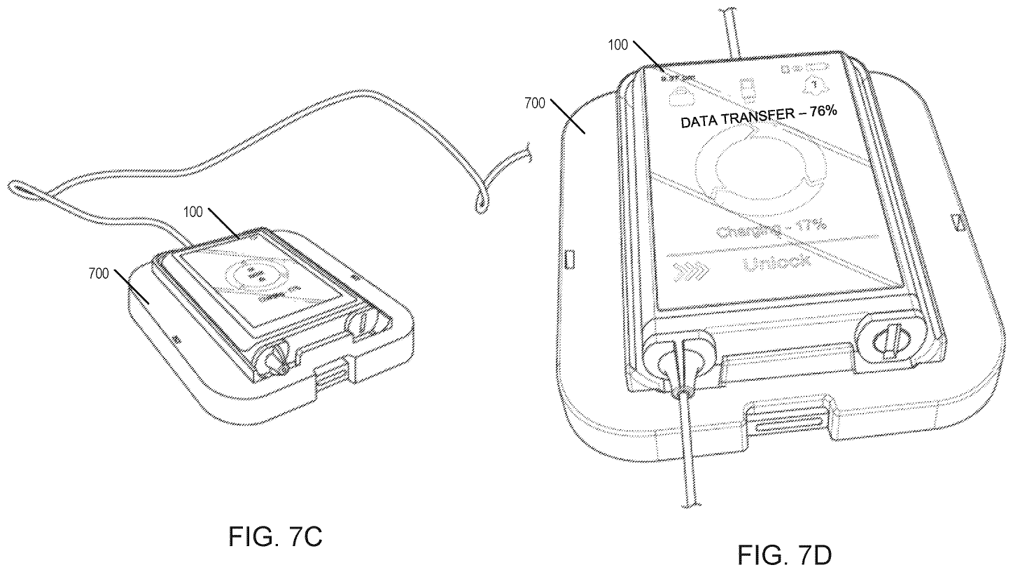

Detailed descriptions and examples of systems and methods according to one or more illustrative embodiments of the present disclosure may be found, at least, in the section entitled Smart Charging Station, and in FIGS. 7A-11 herein. Furthermore, components and functionality for supporting operation of an autonomous glucose level control system that may interact with the charging station may be configured and/or incorporated into the systems and devices described with respect to FIGS. 1-6.

### Infusion System Overview

Some embodiments described herein pertain to medicament infusion systems for one or more medicaments and the components of such systems (e.g., infusion pumps, medicament cartridges, cartridge connectors, lumen assemblies, infusion connectors, infusion sets, etc.). Some embodiments pertain to methods of manufacturing infusion systems and components thereof. Some embodiments pertain to methods of using any of the foregoing systems or components for infusing one or more medicaments (e.g., pharmaceutical, hormone, etc.) to a subject or patient. As an exemplary illustration, an infusion system may include an infusion pump, which can include one or more medicament cartridges or can have an integrated reservoir of medicament. The infusion pump may also be referred to as a medicament pump. An infusion system may include medicament cartridges and cartridge connectors, but not a pump. An infusion system may include cartridge connectors and an infusion pump, but not medicament cartridges. An infusion system may include infusion connectors, a lumen assembly, cartridge connectors, an infusion pump, but not medicament cartridges or an infusion set.

A glucose level control system can operate in conjunction with an infusion system to infuse one or more medicaments, including at least one glucose level control agent, into a subject. The glucose level control agent may include insulin, an insulin analog, a counter-regulatory agent, or any other pharmaceutical or hormone that may affect a subject's glucose level or glucose sensitivity (e.g., metformin, prandin, canagliflozin, etc.).

Any feature, structure, component, material, step, or method that is described and/or illustrated in any embodiment in this specification can be used with or instead of any feature, structure, component, material, step, or method that is described and/or illustrated in any other embodiment in this specification. Additionally, any feature, structure, component, material, step, or method that is described and/or illustrated in one embodiment may be absent from another embodiment.

### Glucose Level Control System Overview

Glucose level control systems are used to control a glucose level (such as, but not necessarily, a blood glucose level) in a subject or patient. In some aspects, the glucose level may comprise a blood glucose level, or a glucose level in other parts or fluids of the subject's body. In some examples, the glucose level may comprise a physiological glucose level of the subject that can be a concentration of glucose in the subject's blood or an interstitial fluid in a part of the subject's body (e.g., expressed in milligram per deciliter (mg/dL or sometimes mg/dl)).

Glucose level control systems (GLCSes), which can also be referred to herein as glucose control systems or glucose level systems, can include a controller configured to generate dose control signals for one or more glucose control agents that can be infused into the subject. Glucose control agents can include regulatory agents that tend to decrease a subject's glucose level, such as insulin and insulin analogs, and counter-regulatory agents that tend to increase a subject's glucose level, such as glucagon or dextrose. In some cases, glucose control agents may include alternative hormones or pharmaceuticals that can affect a subject's glucose level or glucose sensitivity, such as metformin, prandin, canagliflozin, etc. A glucose level control system configured to be used with two or more glucose control agents can generate a dose control signal for each of the agents, or medicaments. In some embodiments, a glucose level control system can generate a dose control signal for an agent even though the agent may not be available for dosing via a medicament pump connected or operatively connected to the subject. Further, in some embodiments, a glucose level control system can generate an indication of a recommended dose of an agent or medicament even though the glucose level control system may not be in communication with or in control of a medicament delivery device (e.g., a medicament pump or medicament pen).

Glucose control agents can be delivered to a subject via subcutaneous injection, via intravenous injection, or via another suitable delivery method. In the case of glucose level control therapy via a medicament pump or an ambulatory medicament pump, subcutaneous injection is most common. An ambulatory medicament pump (AMP) is a type of ambulatory medical device, which is sometimes referred to herein as an ambulatory device, an ambulatory medicament device, a mobile ambulatory device, or an AMD. Ambulatory medical devices include ambulatory medicament pumps and other devices configured to be carried by a subject and to deliver therapy to the subject.

In some embodiments, a glucose level control system can implement one or more algorithms relating to medicament control or glucose level control as discussed herein to provide medicament or glucose level control therapy. In some cases, the glucose level control system may operate, at least in part, without being connected to an ambulatory medicament device. For example, the glucose level control system can provide instructions or output a recommendation of a dose of medicament that directs a user to administer the medicament to provide glucose level control therapy. In some implementations, the user may use, for example, a medicament pen to manually or self-administer the medicament according to the glucose level control system's recommended dose outputs. In some implementations, the user may provide inputs, such as glucose level readings to the glucose level control system. The glucose level control system may generate medicament dose recommendations based at least in part on the user inputs. The user inputs to the glucose level control system may be combined with inputs from other systems or devices, such as sensors as discussed herein. In some implementations, the glucose level control system can provide glucose level control therapy based on user inputs without other system or device inputs.

In some embodiments, the glucose level control system includes a memory that stores specific computer-executable instructions for generating a dose recommendation and/or a dose control signal. The dose recommendation and/or the dose control signal can assist with glucose level control of a subject via medicament therapy. The dose recommendation or dose output of the glucose level control system can direct a user to administer medicament to provide medicament therapy for glucose level control, including manual administration of medicament doses. In additional embodiments, the glucose level control system may include a medicament delivery interface that can interact with a medicament delivery device that delivers at least a portion of the medicament therapy. In further embodiments, the glucose level control system may include a sensor interface that can interact with a sensor configured to generate a glucose level signal corresponding to a glucose level of a subject. The glucose level control system can generate the dose recommendation and/or the dose control signal based at least in part on the glucose level signal. In certain embodiments, the dose recommendation and/or the dose control signal can additionally be based at least in part on values of one or more control parameters. Control parameters can include subject-specific parameters, delivery device-specific parameters, glucose sensor-specific parameters, demographic parameters, physiological parameters, other parameters that can affect the glucose level of the subject, or any combination of one or more of the foregoing.

In some examples, the ambulatory medical device may be an electrical stimulation device, and therapy delivery may include providing electrical stimulation to a subject. An example of an electrical stimulation device is a cardiac pacemaker. A cardiac pacemaker generates electrical stimulation of the cardiac muscle to control heart rhythms. Another example of an electrical stimulation device is a deep brain stimulator to treat Parkinson's disease or movement disorders.

### Example Ambulatory Medicament Device Environment

An ambulatory medicament device (AMD) can be a glucose level control system. The glucose level control system can operate can provide glucose level control via an ambulatory medicament pump connected to a subject. FIGS. 1A-1C illustrate a number of different example configurations for connecting an ambulatory medicament pump to a subject.

FIG. 1A illustrates a first example of an ambulatory medicament device environment for controlling a glucose level of a subject via an ambulatory medicament pump 100. In FIG. 1A, the ambulatory medicament pump 100 is connected to an infusion site 102 using an infusion set 104. The ambulatory medicament pump 100 may have integrated user interface or pump controls 106A that permit a user to view pump data and/or change therapy settings via user interaction with the pump controls 106A, such as via user interface elements. An analyte sensor 110, such as a glucose level sensor or a glucose sensor, generates a glucose level signal that that may be received by a glucose level control system, which may be included as part of the ambulatory medicament pump 100 or may be part of a separate device. The analyte sensor 110 may be a bio sensor. In some variants, the analyte sensor 110 can include an insulin level sensor that can generate an insulin level signal that can be received by the glucose level control system. In some variants, the analyte senor 110 can include a glucose level sensor and/or an insulin level sensor. In some variants, the analyte senor 110 may include a continuous glucose monitor (CGM).

FIG. 1B illustrates a second example of an ambulatory medicament device environment for controlling a glucose level of a subject via an ambulatory medicament pump 100. In FIG. 1B, the ambulatory medicament pump 100 communicates with an external electronic device 108 (such as, for example, a smartphone, tablet, smartwatch, smartglasses, etc.) via a wireless data connection. Like the ambulatory medicament pump ambulatory medicament pump 100, the external electronic device 108 may include pump controls 106B that permit a user to view pump data and/or change therapy settings via user interaction with the pump controls 106B. The pump data may be viewed on a display of the external electronic device 108. Further, at least some of the pump controls 106A can be manipulated via user interaction with user interface elements (e.g., the pump controls 106B) of the external electronic device 108. The analyte sensor 110 (e.g., a glucose level sensor) can also communicate with the ambulatory medicament pump 100 via a wired or wireless data connection. Example user interfaces that can be implemented by one or more of the external electronic devices 108, the ambulatory medicament pump 100, a remote electronic device, and/or other electronic devices are shown and described in International or PCT Publication Nos. WO 2021/067767 and WO 2021/011699, the entire contents of which are hereby incorporated by reference herein and made a part of this specification.

FIG. 1C illustrates a third example of an ambulatory medicament device environment for controlling a glucose level of a subject via an ambulatory medicament pump 100. In FIG. 1C, the ambulatory medicament pump 100 includes an integrated cannula that inserts into the infusion site 102 without a separate infusion set, such as with a patch pump. At least some of the pump controls 106B can be manipulated via user interaction with user interface elements of an external electronic device 108. In some instances, pump controls can be manipulated via user interaction with user interface elements generated by a remote computing environment (not shown), such as, for example, a cloud computing service, that connects to the ambulatory medicament pump 100 via a direct or indirect electronic data connection.

FIGS. 1A-1C illustrate certain non-limiting use cases. It should be understood that other use cases are possible. For example, a glucose level control system of the ambulatory medicament pump 100 may implemented separately from the ambulatory medicament pump 100 itself. In some such cases, the glucose level control system may control the ambulatory medicament pump 100 via an interface, which may be wired or wireless. In some other cases, the glucose level control system may communicate with alternative devices that may be used to facilitate care of the subject. For example, the glucose level control system may communicate with an injection pen or smartpen that a user may use to provide therapy to a subject. As another example, the glucose level control system may communicate with a patch pump that may provide therapy to the subject.

A glucose level control system, which may be part of or may include an ambulatory medicament pump 100 or implemented as a separate system that may be capable of controlling the ambulatory medicament pump 100, may include a user interface configured to provide one or more of therapy information, glucose level information, and/or therapy control elements capable of changing therapy settings via user interaction with user interface controls. For example, the user can provide an indication of an amount of a manual bolus of medicament. The indication may be provided via a user interface of the glucose level control system or from an electronic device remote from the glucose level control system or the ambulatory medicament pump 100. The user interface can be implemented via an electronic device that includes a display and one or more buttons, switches, dials, capacitive touch interfaces, touchscreen interfaces, or other user interface elements. In some embodiments, at least a portion of the user interface is integrated with an ambulatory medicament pump 100 that can be tethered to a body of a subject via an infusion set configured to facilitate subcutaneous injection of one or more glucose level control agents. In certain embodiments, at least a portion of the user interface is implemented via an electronic device separate from the ambulatory medicament pump 100, such as a smartphone

### Example Glucose Level Control System Configurations

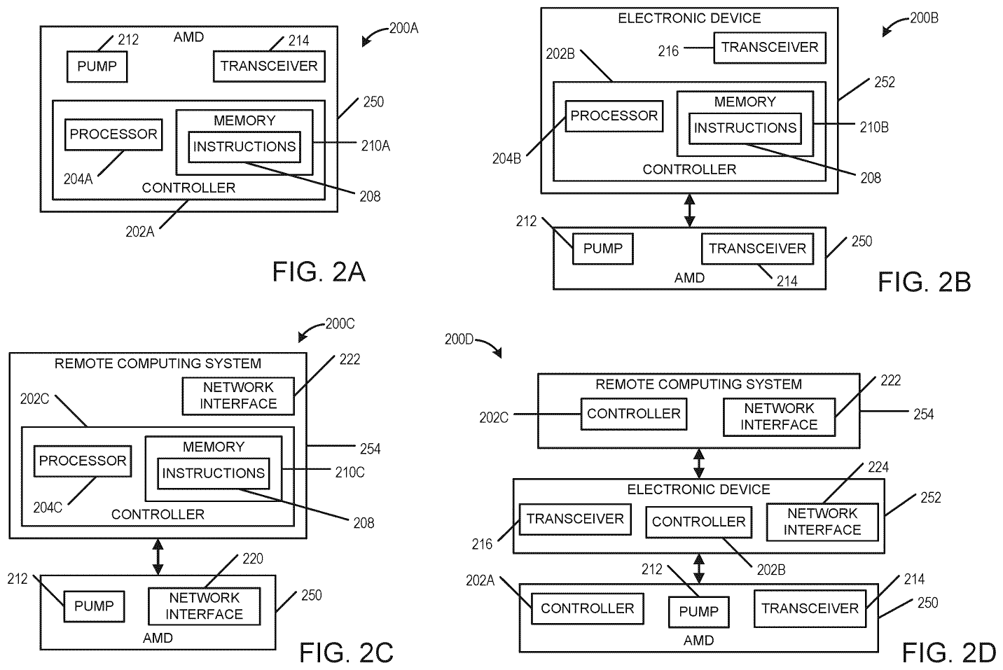

A glucose level control system may be implemented using a number of different configurations that may include a number of different integrated or separate systems. FIGS. 2A-2D illustrate block diagrams of some non-limiting example configurations of a glucose level control system.

FIG. 2A illustrates a block diagram of a first example of a glucose level control system 200A. As illustrated in FIGS. 2A, the glucose level control system 200A can be an ambulatory medicament device 250. The ambulatory medicament device 250 may include a controller 202A. The controller 202A may include an electronic processor 204A and a memory 210A. In some embodiments, the controller 202A may be a separate hardware processor from the electronic processor 204A. Alternatively, the controller 202A may be implemented as software that is executed by the electronic processor 204A.

The memory 210A may be a non-volatile memory, such as flash, a solid-state drive, a hard drive, or any other type of memory that may store instructions 208 executable by the electronic processor 204A. The ambulatory medicament device 250 may further include a medicament pump 212. The medicament pump 212 may include any type of medicament pump that can administer medicament, such as insulin or an insulin analog, to a subject. In some embodiments, the medicament pump 212 may include one or more of the embodiments described with respect to the ambulatory medicament pump 100. When the instructions 208 stored in the memory 210A are executed by the electronic processor 204A, the controller 202A can implement at least a portion of a control algorithm that generates dose control signals for one or more glucose level control agents (e.g., insulin or insulin analogs, or counter-regulatory agents, etc.) based at least in part on time-varying glucose level data corresponding to glucose levels of the subject and/or one or more control parameters. The dose control signals, when delivered to the medicament pump 212, may result in dosing operations to dose medicament that can be used to control the glucose level of a subject.

In some implementations, the ambulatory medicament device 250 may include a transceiver 214. The transceiver 214 may include any type of transceiver that permits or enables wireless communication with another electronic device, such as a router, a smartphone, a laptop, a tablet, a charging station, or any other electronic device that can be configured to communicate with the ambulatory medicament device 250. Using the transceiver 214, the ambulatory medicament device 250 may transmit glucose level data, therapy data (e.g., timing or frequency of medicament delivery), or any other information collected by the ambulatory medicament device 250 to another electronic device.

FIG. 2B illustrates a block diagram of a second example of a glucose level control system 200B. As illustrated in FIG. 2B, in some cases, the glucose level control system 200B can be divided into multiple systems, an ambulatory medicament device 250 and an electronic device 252. The ambulatory medicament device 250 may at least partially be controlled via execution of instructions 208 by the electronic processor 204B of the electronic device 252, which may be separate from an electronic processor 204A of the ambulatory medicament device 250. The electronic device 252 can include a transceiver 216 capable of establishing a wireless connection to the ambulatory medicament device 250 via the transceiver 214 of the ambulatory medicament device 250.

The controller 202B can implement at least a portion of a control algorithm via execution of instructions 208 stored in the memory 210B. When the instructions 208 stored in the memory 210B are executed by the electronic processor 204B, the controller 202B can implement at least a portion of a control algorithm that generates dose control signals for one or more glucose level control agents based at least in part on time-varying glucose level data corresponding to glucose levels of the subject and one or more control parameters. The dose control signals, when delivered to the medicament pump 212, may result in dosing operations that control the glucose level of a subject. The medicament pump 212 may be controlled by at least one pump controller. The pump controller may receive the dose control signals and control the operation of the medicament pump 212 based on the received dose control signals. In some embodiments, the pump controller may be integrated with the medicament pump 212. In various implementations, the controller may be included in the ambulatory medicament device 250, in an electronic device 252 or a remote computing system 254, that are connected to the ambulatory medicament device 250 via wired or wireless communication links. In some embodiments, the dose control signals are transmitted from the transceiver 216 of the electronic device 252 to the transceiver 214 of the ambulatory medicament device 250 over a wired or wireless data connection. The wireless connection may be a short-range wireless connection, such as a Bluetooth® connection, a near-field wireless connection, or any other type of short-range wireless connection. In some cases, the wireless connection may include wide-area communication technologies, such as Wi-Fi or cellular. The ambulatory medicament device 250 may receive the dose control signals over the wireless connection and may provide the dose control signals to the medicament pump 212 to administer medicament to a subject.

In some cases, the electronic device 252 may be a remote system. FIG. 2C illustrates a block diagram of a third example of a glucose level control system 200C that can communicate with a remote computing system 254, such as a cloud or network computing system accessible via a network, such as the Internet. The ambulatory medicament device 250 can be at least partially controlled by instructions 208 executed by an electronic processor 204C of a remote computing system 254. When the instructions 208 stored in a memory 210C of the remote computing system 254 are executed by the electronic processor 204C of the remote computing system 254, the controller 202C can implement at least a portion of a control algorithm that generates dose control signals for one or more glucose level control agents based on time-varying glucose level data corresponding to glucose levels of the subject and one or more control parameters. The dose control signals, when delivered to the medicament pump 212, may result in dosing operations that control the glucose level of a subject. In some embodiments, the dose control signals are transmitted from a network interface 222 of the remote computing system 254 to a network interface 220 of the ambulatory medicament device 250 over an end-to-end wireless data connection. The end-to-end wireless data connection may be any type of wide area network connection. For example, the wide area network may be a cellular network and the end-to-end wireless data connection may be a cellular network connection (e.g., a 4G LTE or 5G network connection). The ambulatory medicament device 250 may receive the dose control signals via the end-to-end wireless data connection and may provide the dose control signals to the medicament pump 212 to administer medicament to a subject.

The ambulatory medicament device 250 and/or the electronic device 252 may include a user interface that provides a cellular network connection activation framework. For example, upon initial use of the ambulatory medicament device 250 and/or the electronic device 252, the user may be guided through steps to activate the ambulatory medicament device 250 and/or the electronic device 252 on the cellular network as part of setting up the ambulatory medicament device 250 and/or the electronic device 252. The ambulatory medicament device 250 and/or the electronic device 252 may include a Subscriber Identity Module (SIM) card or an embedded-SIM (eSIM) that stores information that may be used to identify and authenticate the ambulatory medicament device 250 and/or the electronic device 252 on the cellular network. The SIM card or eSIM may enable the ambulatory medicament device 250 and/or the electronic device 252 to function as an Internet-of-Things (IoT) device that can communicate over a network that supports communication with IoT devices.

The electronic processor 204A, electronic processor 204B, and electronic processor 204C may collectively or individually be referred to as an electronic processor 204. It should be understood that with both a local device, such as the electronic device 252, or a remote device, such as the remote computing system 254, the electronic processor 204 may determine dosing information, which may be provided over a communication connection to the ambulatory medicament device 250. The ambulatory medicament device 250 may generate the dose control signals to control the medicament pump 212 based on the received dosing information. The dose control signals may be generated by a controller or processor included in the ambulatory medicament device 250 as illustrated with respect to the FIG. 2D.

FIG. 2D illustrates a block diagram of a fourth example of a glucose level control system 200D. The glucose level control system 200D may include a plurality of controllers distributed among different elements of the glucose level control system 200D that cooperate to generate a dose control signal to control the medicament pump 212 to administer medicament to a subject. For example, the glucose level control system 200D includes a controller 202A of the ambulatory medicament device 250, a controller 202B of the electronic device 252, and a controller 202C of the remote computing system 254, each of which may function independently or in conjunction with each other to generate dose control signals to control the medicament pump 212.

In the example glucose level control system 200D of FIG. 2D, the remote computing system 254 can transmit or receive data or instructions via the network interface 222, which may establish a network connection with a network interface 224 of the electronic device 252. The network connection may be any through any type of wide area network. For example, the network connection may be a cellular network or any other type of computing network that relies on shared protocols to establish communication between computing systems, such as the remote computing system 254 and the electronic device 252. Further, the electronic device 252 can transmit or receive data or instructions via the transceiver 216, which may establish a communication connection with a transceiver 214 of the ambulatory medicament device 250. The communication connection between the transceivers 214 and 216 may differ from that of the network connection between the network interfaces 222 and 224 in that the communication connection between the transceivers 214 and 216 may be implemented using one or more short-range or near-field communication protocols (e.g., Bluetooth®, Zigbee®, LoRa®, NFC, etc.) while the network connection between the network interfaces 222 and 224 may be implemented using one or more wide-area network protocols (e.g., Fiber-optic communication, cable modem, ISDN, etc.), which may be wired or wireless. In some cases, the wide-area network may include the Internet and/or one or more cellular networks. In certain embodiments, the transceivers 214 and 216 and the network interfaces 222 and 224 may implement the same protocols. In some such cases, the communication connection that may be established between the transceivers 214 and 216 may be of the same type as the network connection between the network interfaces 222 and 224.

In some embodiments, the electronic device 252 can be omitted. In some such cases, the controller 202A and the controller 202C may function independently or in conjunction with each other to generate dose control signals to control the medicament pump 212. In some such embodiments, the ambulatory medicament device 250 may include a network interface (not shown) to support a direct end-to-end wireless data connection, or other network connection, with the remote computing system 254.

The glucose level control system 200A, glucose level control system 200B, glucose level control system 200C, and glucose level control system 200D may collectively or individually be referred to as a glucose level control system 200. Further, in some embodiments, the glucose level control system 200 may be the ambulatory medicament device 250 or may be included as part of the ambulatory medicament device 250. Although illustrated as part of the ambulatory medicament device 250, the medicament pump 212 may be a separate device that is in communication with or capable of being controlled by the ambulatory medicament device 250 or the glucose level control system 200.

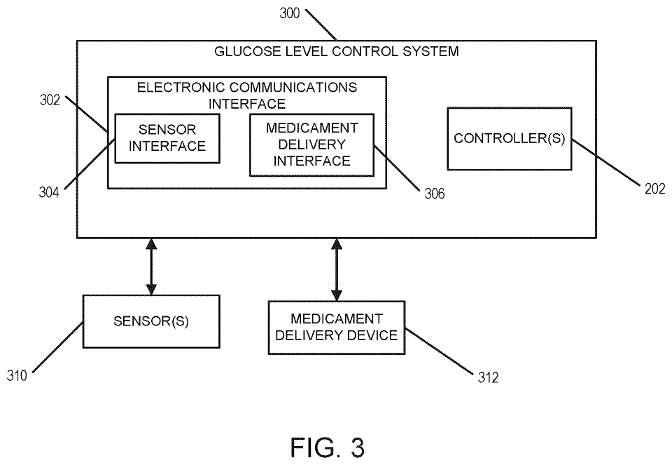

FIG. 3 illustrates a block diagram of an example glucose level control system 300 that includes an electronic communications interface 302. The glucose level control system 300 may include one or more of the embodiments described with respect to any of the glucose level control systems 200.

The electronic communications interface 302 may include any type of circuitry configured to send and receive electronic data from one or more electronic devices. The electronic communications interface 302 may include input/output (I/O) circuitry configured to interface with one or more electronic devices configured to facilitate glucose level control of a subject. For example, the electronic communications interface 302 may include a sensor interface 304 configured to receive a glucose level signal or glucose level data from a glucose level sensor 310, such as a continuous glucose monitor (CGM) sensor. Some CGM sensors may generate glucose level data or a corresponding glucose level signal at fixed measurement intervals, such as at one-minute, five-minute, or ten-minute intervals. The glucose level sensor 310 can be operatively connected to a subject in order to generate a glucose level signal that corresponds to a glucose estimate or measurement of a glucose level of the subject. It should be understood that the sensor interface 304 may interface or communicate with other types of sensors. For example, the glucose level sensor 310 may be replaced with or supplemented by a heart rate monitor or sensor, a motion sensor, a ketone sensor, and/or any other type of physiological sensor or other sensor that can facilitate monitoring a subject's health.

As another example, the electronic communications interface 302 may include a medicament delivery interface 306. The medicament delivery interface 306 may be configured to provide a dose control signal to a medicament delivery device 312. The dose control signal may be generated by a controller 202 (which may represent one or more controllers) based at least in part on a glucose level signal. Alternatively, or in addition, the medicament delivery device 312 may provide an indication of a recommended dose of medicament to the medicament delivery device 312. The medicament delivery device 312 may include any type of device configured to administer (automatically or in response to user-interaction) medicament to a subject. For example, the medicament delivery device 312 may be or may include a medicament pump 212 or an insulin pen.

In some embodiments, the sensor interface 304 may communicate with the glucose level sensor 310, or other sensors, via a short-range wireless connection (e.g., using the transceiver 214). In some embodiments, the sensor can be an insulin level sensor that can detect insulin levels or any other type of analyte sensor The sensor interface 304 can be configured to receive an insulin level signal from the sensor, which can correspond to an insulin level estimate or measurement of the subject (e.g., a concentration of insulin in the subject's blood). The insulin level signal can be used by the one or more controllers discussed herein to generate a dose control signal, which can be provided to the medicament pump 212 via the medicament delivery interface 306. In some embodiments, the sensor can include a glucose level sensor 310 and an insulin sensor.

In some embodiments, the medicament delivery interface 306 may communicate with the medicament delivery device 312 via a short-range wireless connection (e.g., using the transceiver 214). Alternatively, or in addition, the medicament delivery interface 306 may communicate with the medicament delivery device 312 via a local data bus, such as in implementations where the controller 202, the medicament delivery interface 306, and the medicament delivery device 312 are integrated into a single ambulatory medicament device 250. In some cases, the medicament delivery interface 306 may communicate with the medicament delivery device 312 via a wide area network using, for example, the network interface 220.

The controller 202 can be configured to generate a dose control signal using a control algorithm that generates at least one of a basal dose, a correction dose, and/or a food-intake or meal dose. Examples of control algorithms that can be used to generate these doses are disclosed in U.S. Pat. Nos. 7,806,854; 9,833,570; 10,543,313; 10,842,934; 10,940,267; U.S. Patent Publication No. 2021/0213200; and International or PCT Publication No. WO 2012/058694 (referenced herein as the “Controller Disclosures”), the entire contents of which are hereby incorporated by reference herein in their entirety and made a part of this specification. The correction dose can include regulatory (e.g., insulin or an insulin analog) or counter-regulatory agent (e.g., Glucagon) and can be generated using a model-predictive control (MPC) algorithm and/or a biexponential pharmacokinetic (PK) model. Examples of different algorithms and/or models that may be used to generate the correction doses are disclosed in the Controller Disclosures. The basal dose can include regulatory agent and can be generated using a basal control algorithm, such as those disclosed in the Controller Disclosures. The food-intake dose can include regulatory agent and can be generated using a meal control algorithm such as those disclosed in the Controller Disclosures. In some cases, the food-intake dose may be administered in response to any food-intake, food-intake of a particular size, or food-intake associated with particular defined meals. As indicated above, in some cases, the correction dose may include a counter-regulatory agent. However, in other cases, the counter-regulatory agent may be controlled or dosed separately from the correction dose. Additional aspects and improvements for at least some of these controllers are disclosed herein. The dose control signal can be transmitted to a medicament delivery device 312 via the electronic communications interface 302 (e.g., using the medicament delivery interface 306). Alternatively, or in addition, the dose control signal may be transmitted to a medicament pump 212 (or other medicament delivery device 312) via an electrical conductor in implementations where the controller 202 is integrated into the same housing as the medicament pump 212, which may include an infusion motor or other delivery components.

As indicated above, in some cases a glucose level sensor 310 can measure a glucose level of a subject. The control algorithm executed by the controller 202 can determine one or more doses of medicament based at least in part on the glucose level of the subject measured by the glucose level sensor 310 or by an isolated glucose level measurement provided to the controller 202. In some embodiments, the controller 202 may determine a medicament on board value. The medicament on board value may be used by the controller 202 instead of or in addition to the glucose level of the subject to generate a dose control signal using a control algorithm. In other words, in some embodiments, the controller 202 may generate a dose control signal based at least in part on the medicament on board value or the total medicament (e.g., insulin) that is within the subject or that is still active to a minimum degree.

The medicament on board value may correspond to a measurement or an accounting of the total medicament in the subject, in the subject's blood plasma, and/or that is active in the subject's body. The medicament on board value may be obtained by tracking medicament therapy provided to the subject. As different subjects may process medicament (e.g., insulin) differently, the determination of the amount of medicament on board may be a subject-specific determination. Accordingly, in some cases, the medicament on board value may be adjusted based on subject-specific parameters (e.g., weight or sex) or using a learning algorithm that may determine an average time of activity of the medicament for the specific subject using one or more measurements corresponding to the activity of the measurement (e.g., a measure of glucose level at one or more times subsequent to a dose of insulin). Further, as each subject may process medicament differently, in some cases, the medicament on board value may be an estimate of the total medicament within the subject and/or that remains active at a particular point in time. Further, the estimate of medicament on board may be determined based at least in part on total administered medicament, glucose level of the subject, food-intake announcements, activity level, and/or other subject-specific data that may affect the determination of medicament on board.

The medicament on board data value may be based at least in part on one or more basal doses and/or one or more non-basal dose (e.g., a food-intake dose, correction bolus, etc.). In some embodiments, the medicament on board data may correspond with the insulin on board of the subject over a therapy period.

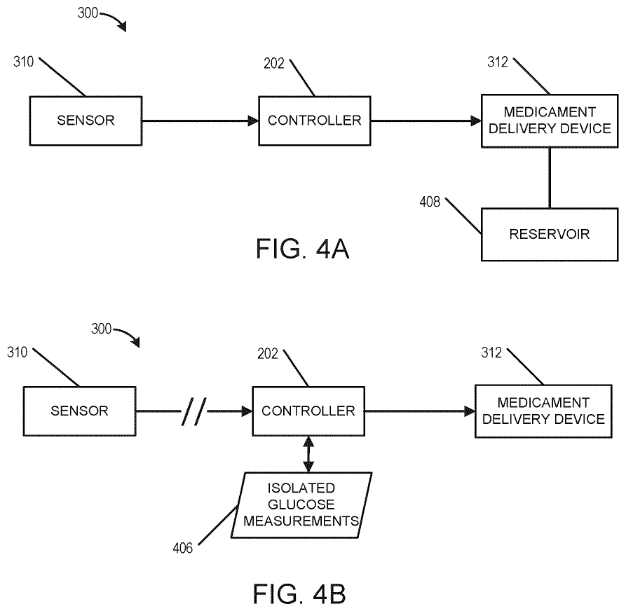

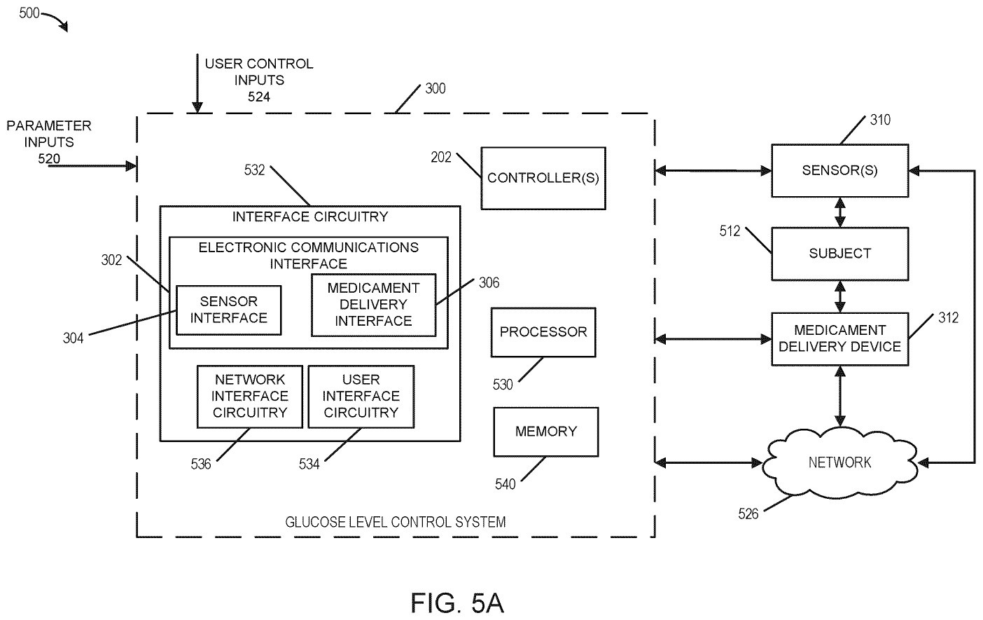

FIG. 4A illustrates a block diagram of an example of elements of a glucose level control system 300 operating in an “online mode.” In the online mode, the controller 202 receives a glucose level signal corresponding to a glucose level of a subject from the glucose level sensor 310. Further, during online mode, a control algorithm implemented by the controller 202 may generate a dose control signal that causes the medicament delivery device 312 to administer a medicament dose. The control algorithm may generate the dose control signal based at least in part on the glucose level signal received from the glucose level sensor 310. Further, the control algorithm may generate the dose control signal based at least in part on control parameters, or values of the control parameters, of the control algorithm. The medicament dose may be a correction dose, a basal dose, or a food-intake dose of medicament. The medicament delivery device 312 may be configured to deliver at least correction doses and basal doses to the subject without substantial or any user intervention when the controller 202 is operating in the online mode. In some examples, the medicament delivery device 312 can include one or more medicament cartridges or can have an integrated reservoir 408 of medicament. The reservoir 408 may be integrated with the medicament delivery device 312. A medicament stored in the reservoir 408 can be delivered to the subject by operation of the medicament delivery device 312. A pump motor of the medicament delivery device 312 can direct medicament for infusion in response to one or more dose control signals as discussed herein. In various embodiments, the operation of the medicament delivery device 312 can be controlled by the controller 202. In some cases, during the online mode, the controller 202 may generate the dose control signal using one or more control schemes described in the Controller Disclosures.

FIG. 4B illustrates a block diagram of an example of elements of a glucose level control system 300 operating in an “offline mode.” In the offline mode, the controller 202 do not receive a glucose level signal from the glucose level sensor 310. The glucose level signal may not be received from the glucose level sensor 310 for a number of reasons (as indicated by the break in the arrow connecting the glucose level sensor 310 block and the controller 202 block in FIG. 4B). For example, the glucose level sensor 310 may not be present or operatively connected to the subject. As another example, the glucose level sensor 310 may be damaged or a communication connection between the glucose level sensor 310 and the controller 202 may have failed or been interrupted. In yet another example, the glucose level sensor 310 may have at least partially become disconnected from the subject. Accordingly, in some cases, the omission of the glucose level signal may be intended and in other cases, the omission of the glucose level signal may be unintended. In other words, in some cases, the glucose level signal may be expected, but not received.

In the offline mode, a control algorithm implemented by the controller 202 may generate a dose control signal that causes the medicament delivery device 312 to administer a medicament dose. However, while the controller 202 may use a glucose level signal received from the glucose level sensor 310 to generate the dose control signal in online mode, in offline mode, the controller 202 may base the dose control signal on an isolated glucose measurement 406 (or a plurality of isolated glucose measurements). The isolated glucose measurements 406 may be based on glucose test strips or other manual measurement technology for obtaining glucose level data corresponding to a glucose level of the subject. In the offline mode, the controller may generate dose control signals using one or more algorithms described in the Controller Disclosures. In the offline mode, the control algorithm may generate a dose control signal that implements correction doses in response to isolated glucose measurements 406 (such as, for example, measurements obtained from the subject using glucose test strips). Alternatively, or in addition, the dose control signals may be generated based at least in part on insulin measurements. Further, the dose control signals may be based on control parameters of the control algorithm. The medicament delivery device 312 may be configured to deliver basal doses to the subject without substantial user intervention and can deliver correction doses to the subject in response to isolated glucose measurements 406 and/or isolated insulin measurements while the controller 202 remains in offline mode. In some embodiments, the controller 202 may operate in “offline mode” during time periods when one or more glucose level signals received from the glucose level sensor 310 are identified as artifacts (e.g., suspected artifacts) as described in PCT Application No. PCT/US2022/027531, the entire contents of which are hereby incorporated by reference in its entirety herein.

Further, as in online mode, during offline mode the control algorithm may generate the dose control signal based at least in part on control parameters, or values of the control parameters, of the control algorithm. Further, as with the online mode, the medicament dose may be a correction dose, a basal dose, or a food-intake dose of medicament. And the medicament delivery device 312 may be configured to deliver at least correction doses and basal doses to the subject without substantial or any user intervention when the controller 202 is operating in the offline mode. However, in contrast to the online mode, additional user intervention may be required to provide the isolated glucose measurements 406 to the controller 202 when operating in the offline mode. In some cases, additional user intervention may not be required during offline mode because, for example, the controller 202 may generate a dose control signal based on glucose level data obtained when the glucose level control system 300 was operating in the online mode or from previously obtained isolated glucose measurements 406. In some such cases, additional user intervention may not be required for a period of time when operating in offline mode. However, after the period of time has elapsed, additional user intervention, such as providing isolated glucose measurements 406 to the controller 202, may be required.

Regardless of whether the glucose level control system 300 is operating in online mode or offline mode, the glucose level control system 300 can operate in an open-loop mode or a closed-loop mode. When operating in the open-loop mode, the glucose level control system 300, using a control algorithm, determines the dose control signal based on one or more inputs to the glucose level control system 300. The one or more inputs may include a glucose level signal and/or one or more isolated glucose measurements 406. Further, the one or more inputs may include an indication of food-intake or characteristics of the food consumed during a food-intake event. In some cases, the one or more inputs may include insulin sensitivity of the subject, characteristics of the insulin absorption of the subject, characteristics of the medicament being administered, or any other characteristics of the subject or medicament that may affect an output of the control algorithm.

When operating in closed-loop mode, the glucose level control system 300, using a control algorithm, determines the dose control signal based on one or more prior outputs of the control algorithm. For example, the dose control signal may be based on prior determined dose control signals or corresponding medicament doses. In some cases, the glucose level control system 300 may determine the dose control signal based on a combination of the prior dose control signals and one or more inputs to the glucose level control system 300, which may include one or more of the inputs described with respect to open-loop mode.

### Example Glucose Level Control System Implementation

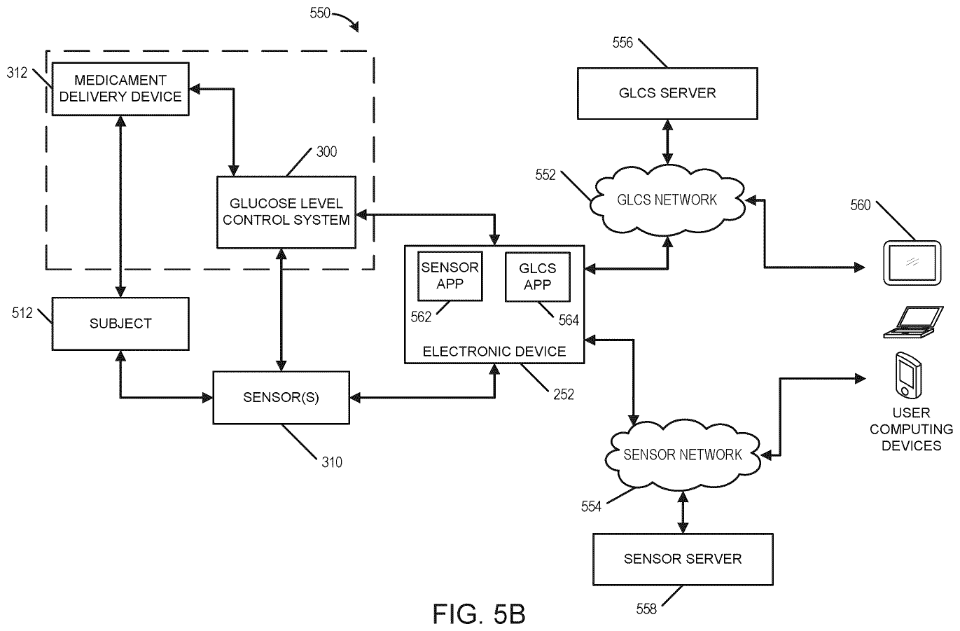

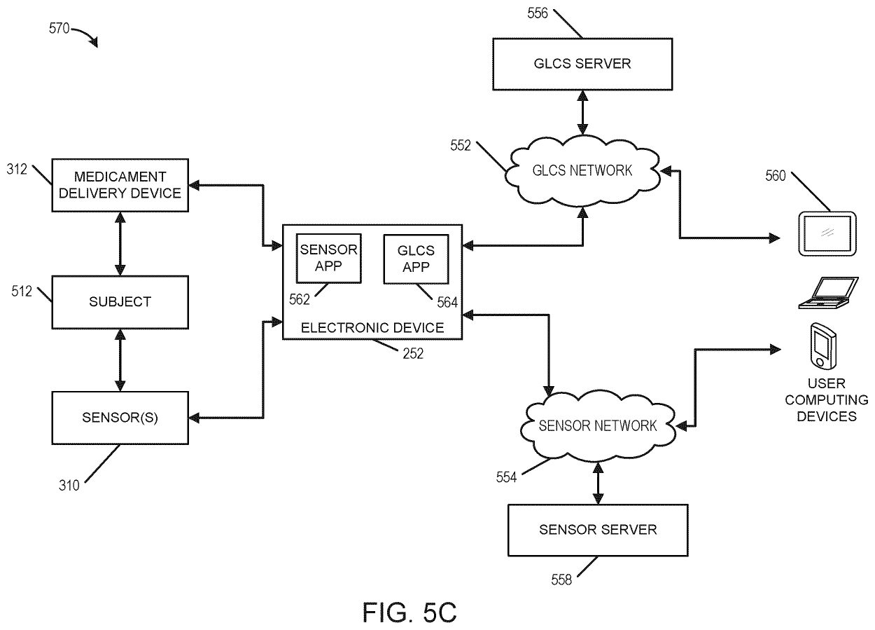

FIG. 5A illustrates a block diagram of an example glucose level control system environment 500 that includes a glucose level control system 300 in accordance with certain embodiments. The glucose level control system 300 can regulate a glucose level (e.g., blood glucose level) of a subject 512. Typically, the subject 512 is a human subject. However, it is possible to adapt the glucose level control system 300 to regulate the glucose level of non-human animals. The glucose level control system 300 may be an example of an automated glucose level regulation system that can regulate the glucose level of the subject 512 with little or no user interaction with the glucose level control system 300. In some cases, a user may interact with the glucose level control system 300 to initialize, setup, or reconfigure the glucose level control system 300, but little to no further user interaction may occur during operation. Alternatively, or in addition, the glucose level control system 300 may be configured to provide manual regulation of the glucose level of the subject. In some cases, the glucose level control system 300 may perform both automatic and manual regulation of the glucose level of the subject. For example, basal and correction doses of medicament may be controlled automatically by the glucose level control system 300 while food-intake doses may be at least partially controlled manually or in response to user interaction with the glucose level control system 300.

The glucose level control system 300 may cause one or more medicament delivery devices 312 to administer one or more doses of insulin to the subject 512. The medicament delivery device 312 may include a medicament pump 212 that can be coupled by a catheter to a subcutaneous space of the subject 512. In some cases, the medicament delivery device 312 may include an insulin pen or medicament patch (e.g., a patch pump), or any other type of device that may administer medicament to the subject 512. As illustrated, the medicament delivery device 312 may be separate, but in communication with the glucose level control system 300. However, in some cases, the medicament delivery device 312 and the glucose level control system 300 may be integrated.

As indicated above, the medicament delivered by the medicament delivery device 312 may be insulin. Alternatively, or in addition, the medicament delivery device 312 may deliver a counter-regulatory agent or hyperglycemic agent, such as glucagon or dextrose, for control of the glucose level under certain circumstances. For the delivery of both insulin and a counter-regulatory agent (e.g., glucagon), the medicament delivery device 312 may be a mechanically driven infusion mechanism having dual cartridges for insulin and the counter-regulatory agent, respectively. In the present description, reference is made to glucagon specifically, but it is to be understood that this is for convenience only and that other counter-regulatory agents (e.g., dextrose) may be used. Similarly, the term “insulin” herein is to be understood as encompassing all forms of insulin-like substances including natural human or animal insulin as well as synthetic insulin in any of a variety of forms (sometimes referred to as “insulin analogs”).

During online and/or autonomous operation of the glucose level control system 300, a glucose level sensor 310 may be operatively coupled to the subject 512. The glucose level sensor 310 may continually sample a glucose level of the subject 512 and the glucose level sensor 310 may be referred to as a continuous glucose monitoring (CGM) sensor. The glucose level sensor 310 may continuously or periodically measure or sense glucose levels of the subject 512 for at least a period of time. The measured or sensed glucose levels of the subject 512 may be blood glucose levels and/or glucose levels of other parts of the subject 512, such as interstitial fluid. Sensing may be accomplished in a variety of ways, generally involving some form of physical coupling between the subject 512 and the glucose level sensor 310. In some embodiments, the glucose level control system 300 may obtain sensor readings from alternative or additional sensors. For example, the glucose level sensor 310 may be supplemented or replaced by additional physiological sensors, such as a heart rate sensor, a motion sensor, or a ketone sensor.

A controller 202 may control operation of the medicament delivery device 312 using a control algorithm. The control algorithm may be implemented or executed as a function of a glucose level signal received from the glucose level sensor 310. Further, the control algorithm may be subject to one or more control parameters that affect the execution and/or output of the control algorithm. In some cases, one or more control parameters may include or may be subject to one or more parameter inputs 520, which may be programmed and/or provided by a user, such as the subject 512, a parent or guardian of the subject 512, or a healthcare provider (e.g., a clinician or doctor). The parameter inputs 520 may include any type of control parameter that can affect implementation or execution of the control algorithm by the controller 202. One example parameter input 520 may include the weight or mass of the subject 512.

In some cases, the glucose level control system 300 can provide effective automated glucose level control without receiving explicit information regarding either meals (or other food-intake) that the subject 512 has ingested or any other “feedforward” information, which may be achieved in part by an adaptive aspect to operation of the controller 202. In other cases, the glucose level control system 300 can use received information regarding either meals (or other food-intake) that the subject 512 ingested, or plans to ingest, or other “feedforward” information to modify control of the glucose level of the subject 512 and/or the delivery of medicament including insulin or counter-regulatory agent.

The controller 202 may be electronic device with control circuitry that provides operating functionality as described herein. In some embodiments, the controller 202 may be implemented using application specific hardware that can execute one or more computer programs including a set of computer instructions. Alternatively, the controller 202 may be implemented using a general-purpose hardware processor configured to execute computer-executable instructions corresponding to one or more computer programs each including respective sets of computer instructions.

In some cases, the glucose level control system 300 may include one or more processors 530, memory 540, and interface circuitry 532. The one or more processors 530, memory 540, and interface circuitry 532 may communicate through a bus (e.g., a set of wires or conductive traces) or other internal communications system that can transfer data or instructions between components of a computer system. The one or more processors 530 may execute one or more sets of computer-executable instructions stored in the memory 540 and configured to control execution of the glucose level control system 300. The one or more processors 530 may control operations and features of the glucose level control system 300 that are separate from the control algorithm configured to maintain or control the glucose level of the subject 512. Alternatively, or in addition, the one or more processors 530 may execute the control algorithm. In such cases, the controller 202 may be integrated with the one or more processors 530 and/or the one or more processors 530 may perform the functionality of the controller 202 and a separate controller 202 may be omitted. The one or more processors 530 may be or may include any type of general purpose processor including, but not limited to a central processing unit (“CPU”), a graphics processing unit (“GPU”), a complex programmable logic device (“CPLD”), a field programmable gate array (“FPGA”), or an application-specific integrated circuit (“ASIC”).

The memory 540 may communicate computer-executable instructions to the one or more processors 530 for execution. The computer-executable instructions may be communicated to the one or more processors 530 via a bus, a cache memory scheme, a direct connection or any other internal communications system. Further, the memory 540 may be further configured to store data associated with operation of the glucose level control system 300 and/or therapy delivered to the subject 512. For example, the memory 540 may store the timing and amount of medicament (e.g., insulin) delivery. The memory 540 can include any type of non-volatile memory and/or volatile memory, such as RAM. The non-volatile memory may include flash memory or solid-state memory. In some cases, the memory 540 may include both main memory (e.g., RAM, ROM, cache, etc.) or primary memory that can store instructions and data actively in use by the one or more processors 530 and long term storage or secondary memory (e.g., magnetic disk memory, optical disk memory, flash, or solid-state memory) that can store applications or data permanently or over a longer term time period than the main memory. While the storage may maintain instructions and data when the glucose level control system 300 is unpowered, the main memory may maintain instructions and data only so long as the glucose level control system 300 is powered.

The interface circuitry 532 can include any type of input/output circuitry that can provide one or more interfaces for interacting with and/or communicating with the glucose level control system 300. For example, the interface circuitry 532 may include the previously described electronic communications interface 302. As previously described, the electronic communications interface 302 may include a sensor interface 304 that can communicate with or receive input from the glucose level sensor 310 and a medicament delivery interface 306 that can communicate with or output commands to the medicament delivery device 312.

The interface circuitry 532 can further include network interface circuitry 536 that enables communication with one or more electronic devices using one or more communication protocols. The interface circuitry 532 can include the transceiver 214, the network interface 220, or any other communication circuitry that enables the glucose level control system 300 to communicate via a wired or wireless connection with an electronic device 252, a remote computing system 254, or any other device. For example, the interface circuitry 532 may include one or more network interface cards and/or wireless radios including, but not limited to, a Bluetooth radio, a Bluetooth Low Energy (BLE) radio, a cellular radio (e.g., a 4G LTE transceiver, a 5G transceiver, or a ND-LTE radio, and the like), an RFID reader, or any other near field, short-range, or wide-area communications circuitry. Further, the interface circuitry 532 may include geolocation hardware, such as a global positioning system (GPS) transceiver. Advantageously, the inclusion of geolocation hardware enables location specific communications, data transfer, and firmware/software upgrades or modifications. Further, the inclusion of geolocation hardware enables location specific control of the glucose level control system 300, such as for detecting the likelihood of exercise (e.g., a swimming facility) that may at least temporarily alter glucose level control therapy. Additionally, the geolocation hardware may be used to facilitate contacting emergency services with location specific information in the event the subject needs help.

As described, the glucose level control system 300 may communicate with one or more of a glucose level sensor 310, a medicament delivery device 312, an electronic device 252, and/or a remote computing system 254, using wired or wireless communication. In some cases, the glucose level control system 300 may, using network interface circuitry 536 and/or the electronic communications interface 302, communicate directly with one or more devices including, but not limited to, the aforementioned devices (e.g., the electronic device 252 or the remote computing system 254). Alternatively, or in addition, the glucose level control system 300 may communicate with the one or more devices via a network 526.

The network 526 may include any type of wired or wireless communication network, including a combination of wired and wireless communication networks. For example, the network 526 may include a local area network (LAN), a wide area network (WAN), a cellular network (e.g., a 4G LTE network, a 5G network, etc.), a wireless LAN (WLAN), an Internet-of-Things (IoT) network, or a combination of networks. Further, in some cases, the network 526 may include the Internet.

The glucose level control system 300 can determine that status information and inputs as discussed herein fail to satisfy one or more safety thresholds. Based on this determination, the glucose level control system 300 or medicament delivery device 312 can automatically generate a distress alert and/or communicate critical events or alarms based on the status information and inputs to the glucose level control system 300 or the medicament delivery device 312. For example, the critical event or alarm may be indicative of a hypoglycemic event or no remaining medicament in the reservoir. The glucose level control system 300 and/or the medicament delivery device 312 can notify the subject, a caretaker, a follower, emergency responders, another user, etc. and/or modify therapy delivery, for example as discussed herein. The glucose level control system 300 and/or the medicament delivery device 312 can track via geolocation data the subject's location and share the location information with a follower and/or emergency personnel.

In some cases, the interface circuitry 532 may include user interface circuitry 534, which may include any circuitry or processors that may output a user interface to a user and/or receive user input from the user via the user interface. The user interface circuitry 534 may receive one or more signals from a processor 530 corresponding to a user interface. The user interface circuitry 534 may control a display to present the user interface to a user based on the one or more signals received from the processor 530. Further, the user interface circuitry 534 may include any circuitry that can receive a signal corresponding to an interaction by a user with a user interface and can provide the signal to the processor 530 and/or controller 202 for further processing.

The user interface circuitry 534 may include any type of output device (e.g., a display screen, audio system, lights, haptic circuitry, etc.) for providing output. Further, the user interface circuitry 534 may include any type of input device (e.g., buttons, dials, keypads, switches, etc.) for receiving input. In some cases, the user interface circuitry 534 may include combined input and output devices, such as a touchscreen controller for controlling a touchscreen or other touch-sensitive device configured to output data and receive input. The touchscreen controller may generate user input signals corresponding to user inputs, such as user control inputs 524. Further, the user interface circuitry 534 can cause user interface screens to be displayed on a display (e.g., a touchscreen display or non-touchscreen display) of the glucose level control system 300. The touchscreen display may accept input via capacitive touch, resistive touch, or other touch-sensitive technology. The user interface circuitry 534 can register the position of one or more touches on a surface of a touch-sensitive display. Further, the user interface circuitry 534 may determine different types of touches or gestures on the touch-sensitive display including, but not limited to, multitouch gestures, taps, multitap inputs (e.g., double or triple taps, etc.), swipes, press and hold interactions, etc.

As described, the user interface circuitry 534 may receive user input in response to interaction with a touchscreen or other touch-sensitive device. Alternatively, or in addition, the user interface circuitry 534 may receive user input via non-touchscreen devices. For example, the glucose level control system 300 may include an alphanumeric keypad (which may include letters, numbers, symbols, or other keys), one or more physical buttons, one or more switches, one or more knobs, or any other type of user interface element that enables a user to interact with the glucose level control system 300. The user interface circuitry 534 may process one or more signals or inputs received via the one or more user interface elements of the glucose level control system 300, or user interface elements of another device (e.g., an electronic device 252) in communication with the glucose level control system 300. In some embodiments, the user interface circuitry 534 may include a sleep and/or wake feature that enables a user to place the glucose level control system 300 into a sleep mode or to wake from a sleep mode. The sleep mode may deactivate a user interface and/or one or more additional features of the glucose level control system 300 enabling a reduction in power consumption and/or preventing inadvertent changes to a configuration of the glucose level control system 300.

In some embodiments, the interface circuitry 532 may include an audio or voice recognition system (e.g., a speaker, a microphone, an interactive voice response (IVR) system, or the like) for receiving an audio or voice command, or other utterance from a user. In some cases, the user interface circuitry 534 may be configured to output via a speaker a sound or voice that may be prestored or generated in real time.

In some implementations, one or more of the interfaces may be provided by the one or more processors 530 or may be provided by the interface circuitry 532 in conjunction with the one or more processors 530. Further, in some cases, one or more of the interfaces (e.g., a user interface) may be provided by or in conjunction with an electronic device 252 or a remote computing system 254. For example, a user (e.g., the subject 512, a parent or guardian, or a clinician or other healthcare provider) may interact with a smartphone, a smartwatch, a tablet, a desktop, or a laptop, and the like, to provide input to the glucose level control system 300 or to access output generated by the glucose level control system 300.

User interaction with the glucose level control system 300 may be performed by the subject 512. Alternatively, or in addition, user interaction may be performed by a user that is separate from the subject 512. For example, a parent or guardian may interact with the glucose level control system 300 on behalf of a child or other subject that may not be capable of configuring the glucose level control system 300 without assistance. In another example, a healthcare provider may assist the subject 512 in configuring the glucose level control system 300 based on subject-specific parameters and/or to help train the subject 512 in maintaining his or her disease.

In some cases, the controller 202 may perform some or all of the functionality of the glucose level control system 300. In some such cases, the processor 530 may be optional or omitted. In other cases, the controller 202 may at least perform automated glucose level control of the subject 512 and/or may generate recommended medicament doses to facilitate manual glucose level control or hybrid glucose level control that includes both automated and manual glucose level control of the subject 512, while one or more separate processors 530 may perform one or more additional operations of the glucose level control system 300. These additional operations may include any features the glucose level control system 300 can provide in addition to the glucose level control provided by the controller 202. For example, the additional operations may include tracking or logging of occurrences of hyperglycemic or hypoglycemic events or risk events, outputting data to a user (e.g., the subject 512, a parent or guardian, or a clinician or other healthcare provider), controlling or initiating communication with another computing system (e.g., the electronic device 252 or the remote computing system 254), regulating access to the glucose level control system 300, or other operations unrelated to operation of a medicament pump 212 or the medicament delivery device 312.

As described above, the controller 202 is capable of operating in an online mode in which the controller 202 receives a glucose level signal corresponding to a glucose level of a subject 512 from the glucose level sensor 310. Alternatively, or in addition, the controller 202 can operate in an offline manner in which the controller 202 does not receive the glucose level of the subject 512 from the glucose level sensor 310. In some such cases, the controller 202 may cause the medicament delivery device 312 to deliver insulin (and potentially glucagon as well) independent of or without receipt of glucose levels from the glucose level sensor 310. In offline mode, the controller 202 may determine medicament doses based at least in part on isolated glucose measurements 406, past glucose level measurements from the glucose level sensor 310, historical therapy data (e.g., past medicament delivery), user input (e.g., food-intake announcements or manual medicament requests), or other control information that may be used to facilitate automatic operation of the glucose level control system 300 for at least a period of time. In some cases, the controller 202 may cause the medicament delivery device 312 to automatically deliver medicament for at least a period of time when operating in the offline mode. Alternatively, or in addition, the controller 202 may output a recommendation of medicament delivery when operating in the offline mode enabling a user to determine whether to deliver the recommended medicament (e.g., insulin, insulin analog, or glucagon) or to confirm whether delivery by the medicament delivery device 312 is to proceed. In some cases, the controller 202 may cause automatic medicament delivery for a period of time and subsequent to the period of time may cease providing automatic delivery of medicament. Cessation of automatic delivery of medicament may occur because, for example, data available for determining medicament delivery may become stale or too old to safely determine medicament dosing.

The offline operating mode of the glucose level control system 300 may be triggered in response to determining that the glucose level sensor 310 needs replacing, is not properly connected to the subject 512, is defective, that a signal is not being received from the glucose level sensor 310, or that the glucose level sensor 310 is absent. Further, in some cases, offline mode may occur in response to a user command, a determination of a lack of available medicament, or a malfunction of the glucose level control system 300. In some embodiments, operation of the glucose level control system 300 may be divided between online periods each including a succession of sampling intervals when a glucose level signal is available, and offline periods each including a succession of sampling intervals when the glucose level signal is either completely or intermittently unavailable. Although “online” and “offline” modes are generally defined by the availability of the glucose level signal, in some cases, offline operation may be user-selected even when a glucose level signal is available to the glucose level control system 300.

In some embodiments, a user (e.g., a subject 512, parent or guardian, healthcare provider, or any other user who may be authorized to help manage therapy of the subject 512, etc.) may provide one or more user control inputs 524 to the glucose level control system 300. The user control inputs 524 may be provided via a user interface generated by the user interface circuitry 534 or a user interface of another device (e.g., an electronic device 252 or remote computing system 254) in wired or wireless communication with the glucose level control system 300. The user control inputs 524 may be provided via a local or remote user interface. In some embodiments, the user interface may resemble that of an insulin pump or similar devices, e.g., by including control buttons for commanding the delivery of a medicament bolus and/or a display. In some embodiments, the glucose level control system 300 may have a wired or wireless interface to a remote device (e.g., an electronic device 252 or a remote computing system 254) that may incorporate a full-function user interface, such as a smartphone, smartwatch, laptop computer, desktop computer, cloud computing service, or other wearable device or computing device. In some embodiments, the wireless interface may provide access to a local area network, such as a personal home network, a company network, or otherwise. Alternatively, or in addition, the wireless interface may provide a direct connection between local devices available to a user (e.g., via Bluetooth or other near field communication technologies). In some cases, the wireless interface may provide access to a wide area network, such as, but not limited to, the Internet. For example, the wireless interface may include a cellular interface that permits access to a network via a 4G or 5G cellular connection. In some cases, the cellular interface may be a low power interface, such as narrowband LTE or other Internet of Things (IoT) interfaces.

As previously described, the one or more parameter inputs 520 may include any input that modifies or corresponds to a control parameter used by the controller 202 to execute a control algorithm for determining whether to administer medicament, the quantity of medicament to administer to the subject 512, and/or the timing of medicament delivery. The user control inputs 524 may include any type of input relating to additional features or control of the glucose level control system 300. In some cases, the user control inputs 524 may include inputs that cause or relate to operation of the controller 202. For example, the user control inputs 524 may include inputs relating to food-intake or a manual request for medicament delivery. Additional non-limiting examples of the user control inputs 524 may include inputs relating to alarm settings, security settings, authorization of one or more users to access data or features of the glucose level control system 300, data delivery configurations, medicament cartridge installation, site changes or setup for connecting the medicament delivery device 312 to the subject 512, user interface preferences, location settings, or any other configuration options of the glucose level control system 300. The glucose level control system 300 can receive user control inputs 524 and/or parameter inputs 520 (e.g., therapy optimization parameters) continuously or periodically via the user interface circuitry 534 (e.g., from user interaction with a user interface of the glucose level control system 300) or the network interface circuitry 536 from the network 526 and/or other healthcare proxy systems or network.

In some cases, a first glucose level control system 300 can transmit (e.g., via the network 526) to a server (e.g., the glucose level control system server 556 of FIG. 5B) historical pump data associated with the delivery of glucose control therapy (e.g., an amount of a medicament delivery, a time of the medicament delivery, etc.) and/or one or more glucose control parameters. Examples of glucose control parameters can include an insulin decay rate constant associated with a decay rate of insulin at a subcutaneous depot of the subject, a clearance time associated with an estimate of an amount of time for a bolus of insulin to be utilized by the subject, an insulin rise rate constant associated with a rise rate of insulin in blood of the subject after a bolus of insulin, a half-life value associated with when a concentration of insulin in blood plasma of the subject reaches half of a maximum concentration in the blood plasma, a weight of the subject, an age of the subject, demographic information associated with the subject, a sensitivity constant (e.g., an indication of a date of diagnosis, which may indicate a newly diagnosed diabetic) associated with the subject's sensitivity to a glucose level or bolus of medicament, a health state the subject (e.g., subject is pregnant or sick), an activity level of subject, a diet of the subject, a basal rate of medicament delivery associated with the subject, a correction factor, a carbohydrate ratio, a glucagon control parameter, and/or any other parameter described herein. The server can transmit to a second glucose level control system 300 (e.g., a replacement glucose level control system 300, such as a replacement ambulatory medicament device 250 and/or replacement electronic device 252) via the network 526 the therapy data and/or one or more glucose control parameters to initialize therapy administration by the second glucose level control system based on the therapy data and/or one or more glucose control parameters from the first glucose level control system.

In some cases, the first and second glucose level control systems 300 can communicate directly with each other (e.g., without the network 526 and/or without an intermediary server) to transmit the therapy data and/or one or more glucose control parameters via near-field communication protocols discussed herein. For example, first and second electronic devices 252 of first and second glucose level control systems 300, respectively, can communicate directly with each to transfer data/parameters via near-field communication protocols. In some cases, the second glucose level control system 300 can receive a configuration code based on the therapy data and/or one or more glucose control parameters to initialize therapy administration by the second glucose level control system 300. In some cases, the first glucose level control system 300 can transfer the therapy data and/or one or more glucose control parameters, including a configuration code, onto a removable data module in communication with first glucose level control system 300. The second glucose level control system 300 can receive the therapy data and/or one or more glucose control parameters, including the configuration code, from the removable data module that is connected to the second glucose level control system 300.

In an offline mode discussed herein, the glucose level sensor 310 may be absent, non-functioning, or not operatively coupled to the subject 512. As such, in the offline mode, the glucose level signal may not be available to control automatic operation. In some cases, a user may provide one or more glucose level measurements to the glucose level control system 300 to facilitate automatic operation of the glucose level control system 300. These measurements may be provided over a particular time period. Alternatively, or in addition, the glucose level control system 300 may use a therapy history and/or a history of prior glucose level control measurements to facilitate automatic operation of the glucose level control system 300 for at least a particular time period.

FIG. 5B illustrates a block diagram of a second example glucose level control system environment 550 in accordance with certain embodiments. As with the glucose level control system environment 500, the glucose level control system environment 550 includes a glucose level control system 300. Further, the glucose level control system environment 550 includes a medicament delivery device 312. The glucose level control system 300 may communicate with the medicament delivery device 312 enabling the glucose level control system 300 to control medicament delivery by the medicament delivery device 312 to the subject 512. Further, the glucose level control system 300 may obtain therapy data (e.g., a size of medicament delivered, status of a medicament cartridge, etc.) from the medicament delivery device 312. As illustrated in glucose level control system environment 550, the glucose level control system 300 and the medicament delivery device 312 may be separate devices. Alternatively, the glucose level control system 300 may be incorporated as part of the medicament delivery device 312 or vice versa, the medicament delivery device 312 may be incorporated as part of the glucose level control system 300. Thus, optionally, the glucose level control system 300 and the medicament delivery device 312 may be replaced by a single system as illustrated by the dashed line box.

The glucose level sensor 310 may be operatively connected to the subject 512 and may measure a glucose level of fluid (e.g., blood or interstitial fluid) of the subject 512. The glucose level sensor 310 may generate a signal corresponding to the measured glucose level and may provide the signal to the glucose level control system 300, which may control the medicament delivery device 312 based at least in part on the sensor signal.

In some embodiments, one or both of the glucose level control system 300 and the glucose level sensor 310 may communicate with an electronic device 252. For example, the glucose level sensor 310 may provide sensor data (e.g., glucose level data) to the electronic device 252. As another example, the glucose level control system 300 may provide to the electronic device 252 historical therapy data corresponding to therapy provided to the subject 512 by the glucose level control system 300 and/or medicament delivery device 312. The electronic device 252 may include any type of computing device that can communicate directly or via a wired or wireless network with the glucose level control system 300. Further, in some cases, the electronic device 252 may include any type of computing device that can execute one or more computer-executable instructions or applications that can control, at least in part, the glucose level control system 300 and/or the medicament delivery device 312. For example, the electronic device 252 may include a smartphone, a smartwatch, a tablet, a laptop, a pair of smartglasses, an application specific controller, and the like.

In some embodiments, the electronic device 252 may host one or more applications that may facilitate accessing data of one or more of the glucose level sensor 310, the glucose level control system 300, and the medicament delivery device 312. Further, the one or more applications may enable the electronic device 252 to at least partially control one or more of the glucose level sensor 310, the glucose level control system 300, and/or the medicament delivery device 312. For example, the electronic device 252 may include a sensor application 562 and/or a glucose level control system (GLCS) application, which may be referred to as a GLCS application 564. In some cases, the sensor application 562 and the GLCS application 564 may be capable of communicating with each other. Alternatively, the sensor application 562 and the GLCS application 564 may be incorporated into a single application. In other words, one application may implement the features described herein of both the sensor application 562 and the GLCS application 564.

The sensor application 562 may include any application that can receive data from the glucose level sensor 310. For example, the sensor application 562 may receive a glucose level signal from the glucose level sensor 310 enabling the sensor application 562 to determine a corresponding glucose level of the subject 512 operatively connected to the glucose level sensor 310. The sensor application 562 may display, or cause to be displayed by the electronic device 252, the data received from the glucose level sensor 310 (e.g., a glucose level signal) and/or data corresponding to the received data (e.g., a glucose level). Further, the sensor application 562 may cause the electronic device 252 to transmit the raw data received from the glucose level sensor 310 and/or data determined from the raw data to a third-party system, such as a sensor server 558. The data may be transmitted to the sensor server 558 via a sensor network 554.

The sensor server 558 may include any system that can receive and/or store data obtained from the sensor application 562. The data may correspond to sensor readings of the glucose level sensor 310 and/or data generated by the sensor application 562 in response to sensor readings by the glucose level sensor 310. For example, the data may include glucose level signals, glucose levels, sensor status information (e.g., errors, length in use, remaining expected usage time, etc.) associated with the glucose level sensor 310, a type or model of the glucose level sensor 310, or any other data that may be obtained by the glucose level sensor 310 and/or associated with operation of the glucose level sensor 310.

The sensor network 554 can include any type of computing or communications network that enables communication between the electronic device 252 and the sensor server 558. In some cases, the sensor network 554 may be or may include a third-party network, such as a network that may be associated with a third-party that manages or maintains the sensor server 558. Alternatively, or in addition, the sensor network 554 may include the Internet and/or the network 526. In some cases, the electronic device 252 may access the sensor network 554 via the network 526 and/or the Internet.

The GLCS application 564 may include any application that can receive data from the glucose level control system 300. The data may include any type of data that may be determined by the glucose level control system 300 including, but not limited to, data associated with therapy provided by the medicament delivery device 312 and data associated with maintaining the disease of the subject 512. For example, the data may include therapy data associated with the timing and quantity of medicament administered to the subject 512. Further, the data may include data associated with the status of the subject 512 and/or the diseases of the subject 512, data associated with the status of the medicament delivery device 312 and/or the glucose level control system 300, and any other data that may be generated or maintained by the glucose level control system 300 and/or the medicament delivery device 312. In some cases, the data may include data obtained from the glucose level sensor 310. In other words, in some cases, the GLCS application 564 may obtain data from the glucose level sensor 310 via the sensor application 562, the glucose level sensor 310 directly, and/or via the glucose level control system 300.

In some cases, the GLCS application 564 may be further configured to control at least in part the glucose level control system 300, which may in turn control the medicament delivery device 312. Alternatively, or in addition, the GLCS application 564 may implement at least some of the features of the glucose level control system 300. In some such cases, the GLCS application 564 may function as the glucose level control system 300. In such cases, the GLCS application 564 may directly control the medicament delivery device 312. In other words, the GLCS application 564 may serve as the glucose level control system 300 and may communicate with the medicament delivery device 312 via the electronic device 252.

Further, the GLCS application 564 may display, or cause to be displayed by the electronic device 252, the data received from the glucose level control system 300 and/or data corresponding to the received data. Moreover, the GLCS application 564 may receive and/or display similar data as the sensor application 562 (e.g., sensor data received from the glucose level sensor 310). In addition, much like the sensor application 562, the GLCS application 564 may cause the electronic device 252 to transmit the raw data received from the glucose level control system 300 and/or data determined from the raw data to a third-party system, such as a GLCS server 556. The data may be transmitted to the GLCS server 556 via a GLCS network 552. In some cases, the sensor application 562 and the GLCS application 564 may be combined or part of the same application. In such cases, the combined application may be capable of communicating with one or both of the GLCS server 556 and the sensor server 558 via the sensor network 554, the GLCS network 552, and/or the network 526.

The GLCS server 556 may include any system that can receive and/or store data obtained from the GLCS application 564. The data may correspond to data generated by the medicament delivery device 312, the glucose level control system 300, the GLCS application 564, the glucose level sensor 310, the sensor application 562 and/or the GLCS application 564. For example, the data may include glucose level signals, glucose levels, sensor status information (e.g., errors, length in use, remaining expected usage time, etc.) associated with the glucose level sensor 310, a type or model of the glucose level sensor 310, therapy data (e.g., timing or quantity of medicament doses), status information associated with the medicament delivery device 312, status information associated with the glucose level control system 300, user interaction history with the glucose level control system 300, the medicament delivery device 312, the glucose level sensor 310, the sensor application 562, or the GLCS application 564, or any other data that may be obtained by the glucose level sensor 310, the medicament delivery device 312, the glucose level control system 300, the sensor application 562, and/or the GLCS application 564.

The sensor network GLCS network 552 can include any type of computing or communications network that enables communication between the electronic device 252 and the sensor application 562. In some cases, the GLCS network 552 may be or may include a third-party network, such as a network that may be associated with a third-party that manages or maintains the GLCS server 556. Alternatively, or in addition, the GLCS server 556 may include the Internet and/or the network 526. In some cases, the electronic device 252 may access the GLCS server 556 via the network 526 and/or the Internet.

In some embodiments, the sensor network 554 and the GLCS network 552 may be the same network. Further, in some cases, the sensor server 558 and the GLCS server 556 may be managed by the same party. Further, one or more of the sensor server 558, the GLCS server 556, the glucose level sensor 310, the medicament delivery device 312, the glucose level control system 300, the electronic device 252, the sensor application 562, and/or the GLCS application 564 may be managed, developed, and/or produced by the same party.

In some cases, the GLCS server 556 and/or the sensor server 558 may provide access to data provided by the electronic device 252 and/or data generated based at least in part on the data provided by the electronic device 252. The data access may be provided to the subject 512 and/or to other users associated with the subject 512 and/or authorized to access data associated with the subject 512. For example, parents, guardians, healthcare providers, or other authorized parties may access the data provided to or generated by the GLCS server 556 or the sensor server 558 using a user computing device 560. The user computing device 560 may include any type of computing device capable of accessing the GLCS server 556 and/or the sensor server 558 via a network, such as the sensor network 554, the GLCS server 556, the network 526, and/or the Internet.

Advantageously, the ability of authorized users (e.g., a parent, guardian, or healthcare provider) to access the GLCS server 556 and/or the sensor server 558 enables the authorized users to help monitor and maintain the disease and/or therapy of the subject 512. Moreover, the authorized users can determine whether the subject 512 requires assistance to help manage the disease of the subject 512.

In some cases, the glucose level control system 300 (e.g., executing the sensor application 562 and/or the GLCS application 564) may periodically transmit data to the GLCS network 552 or the sensor network 554 based on a regular schedule (e.g., every 1, 5, 10, or 20 minutes, once a day, once a week, once a month etc.). Alternatively, or in addition, the data may be transmitted in response to a command (e.g., on demand data transfer) or when the glucose level control system 300 determines it is within a certain location. For example, when the glucose level control system 300 determines it is within a subject's home or at a healthcare provider's office based on a local area network connection or based on a geolocation system (e.g., a global positioning system (GPS)). In some cases, the glucose level control system 300 transmits additional encrypted data on an intermittent basis. Alternatively, or in addition, the glucose level control system 300 transmits additional encrypted data on a continuous basis for at least a time period. The glucose level control system 300 may be configured to transmit data as it is generated, or shortly thereafter, (e.g., in real or near real-time (e.g., within a few millisecond, seconds, or minutes of the data being generated)), or in bulk at specified periods of time.

In some cases, the user computing device 560 may include a follow application. The follow application can allow authorized users to access data provided to or generated by the glucose level control system 300 or the electronic device 252 via the GLCS network 552, the sensor network 554, or the network 526. For example, the follow application can allow authorized users to view in real-time glucose levels/physiological measurements of the subject or doses of medicament (e.g., numerical data or charts/graphs). The follow application executed, for example, on the user computing device 560 can also generate alarms, alerts, or critical events for the authorized user. The follow application can mirror or replicate the display or screen on, for example, the electronic device 252, including mirroring alarms, alerts, or critical events so that, for example, the authorized user sees and has access to the same information on the user computing device 560 as the subject on the electronic device 252. In addition, or alternatively, an authorized user can receive notifications of alarms, alerts, or critical events, such as text messages, or other forms of visual and audio alerts.

The follow application on the user computing device 560 may have different modes that allow the authorized user to select what is replicated or displayed for the authorized user on the user computing device 560. For example, the follow application may replicate the display or screen on the electronic device 252 as-is; or the follow application may generate specific, predetermined alarms, alerts, or critical events or just display glucose levels/physiological measurements of the subject or doses of medicament, which can be set by the authorized user via the follow application. The follow application on the user computing device 560 may have access to or generate therapy reports based on the information received via the GLCS network 552, the sensor network 554, or the network 526. In some cases, the follow application on the user computing device 560 may receive data directly from the electronic device 252 (e.g., without the GLCS network 552, the sensor network 554, or the network 526) via near-field communication protocols discussed herein.

In some cases, user control inputs 524 may be received from a user computing device 560 to, for example, modify control parameters for glucose level control therapy administered or delivered by the medicament delivery device 312. The user control inputs 524 may be received via the follow application or another user interface on the user computing device 560. To provide user access to one or more therapy change controls of a medicament delivery device 312 without putting the health of a subject at risk, access to therapy change controls may be managed using a plurality of safe access levels for the medicament delivery device 312. Each of the safe access levels may correspond to one or more control parameters that can be modified by a user or a subject who is qualified to modify the one or more control parameters and/or is associated with the corresponding safe access level. In some cases, access to the selected control parameters may be allowed by enabling access to the corresponding therapy change controls in the medicament delivery device 312 (e.g., activating or displaying one or more therapy change control elements on a touchscreen user interface on the glucose level control system 300 and/or medicament delivery device 312). The user computing device 560 may communicate with the medicament delivery device 312 via the GLCS network 552, the sensor network 554, or the network 526 to provide user control inputs 524. In some cases, the user computing device 560, such as a remote computing system 254 discussed herein, may be in direct communication with the medicament delivery device 312, such as an ambulatory medicament device 250 discussed herein, (e.g., without the GLCS network 552, the sensor network 554, or the network 526) to provide user control inputs 524 via near-field communication protocols discussed herein.

In some cases, safe access levels may be categorized based on the capacity of a user for modifying control parameters without causing barm or increasing the risk of harmful effects to the subject. For example, safe access levels may be defined based on age, training, experience, and the like. In other cases, safe access levels may be defined or categorized based on a therapy history of the subject. For example, safe access levels may be defined based on certain events or behaviors identified in the therapy data collected in the previous therapy periods when the subject was receiving therapy from the ambulatory medicament device 250. In some cases, one or more safe access levels may be defined based on a desire to modify therapy settings when the medicament delivery device 312 does not provide therapy to human subjects and/or is used to perform investigative research. For example, a safe access level may be dedicated to users who use the medicament delivery device 312 for medical research and another safe access level may be dedicated to users who develop applications for the medicament delivery device 312.

In some cases, the electronic device 252 (e.g., executing the sensor application 562 and/or the GLCS application 564) may periodically transmit data to the GLCS network 552 or the sensor network 554 based on a regular schedule (e.g., every 1, 5, 10, or 20 minutes, once a day, once a week, once a month, etc.). Alternatively, or in addition, the data may be transmitted in response to a command (e.g., on demand data transfer) or when the electronic device 252 determines it is within a certain location. For example, when the electronic device 252 determines it is within a subject's home or at a healthcare provider's office based on a local area network connection or based on a geolocation system (e.g., a global positioning system (GPS)). In some cases, the electronic device 252 transmits additional encrypted data on an intermittent basis. Alternatively, or in addition, the electronic device 252 transmits additional encrypted data on a continuous basis for at least a time period. The electronic device 252 may be configured to transmit data as it is generated, or shortly thereafter, (e.g., in real or near real-time (e.g., within a few millisecond, seconds, or minutes of the data being generated)), or in bulk at specified periods of time. Transmitting the data in bulk at particular time periods may extend battery life, but may provide for less up-to-date analysis. Data can be made available on-demand by keeping the transceiver always on, but this may consume more power. Thus, the scheduling of data transfer may be balanced based on different considerations, such as: (1) power consumption and (2) need to share information with authorized users or systems. The hardware and software of the devices and systems described herein and associated data transfer protocols can be optimized power consumption while meeting the data transfer needs and requirements. The battery size of the various devices of the system can be optimized. For example, with a network integrated pump, it may be more desirable to have a larger or increased battery sized relative to when the system utilizes an electronic device to communicate with the network as discussed herein.

The electronic device 252, glucose level control system 300, and/or user computing device 560 may be used to initiate (e.g., by a user) data downloads and/or firmware/software updates, for example, to the electronic device 252, glucose level control system 300, and/or medicament delivery device 312 as well as to the user computing device 560. In some cases, the data downloads and/or firmware/software updates may be initiated automatically to the electronic device 252, glucose level control system 300, and/or medicament delivery device 312 as well as to the user computing device 560. The electronic device 252, glucose level control system 300, and/or user computing device 560 may be used to initiate (e.g., by a user) data transfer to, for example, customer service and/or manufacturer. In some cases, electronic device 252, glucose level control system 300, and/or user computing device 560 data transfer may include ordering of various disposables used for glucose level control therapy, such as additional supply of medicament, infusion sets, sensors, transmitters, etc.

FIG. 5C illustrates a block diagram of a third example glucose level control system environment 570 in accordance with certain embodiments. In contrast to the glucose level control system environment 550, the glucose level sensor 310 of the level control system environment 570 communicates with the electronic device 252 without communicating with the medicament delivery device 312. In some such cases, the electronic device 252 may communicate information or data (e.g., a glucose level signal or data derived from the glucose level signal) with the medicament delivery device 312 enabling the medicament delivery device 312 to provide therapy based at least in part on the measurements made by the glucose level sensor 310. Alternatively, or in addition, the electronic device 252 using, for example, the GLCS application 564 may generate control signals based at least in part on the glucose level sensor 310 measurements and may transmit the control signals to the medicament delivery device 312 thereby enabling the electronic device 252 to control the medicament delivery device 312 based at least in part on measurements of the glucose level sensor 310. In other words, in certain embodiments, the medicament delivery device 312 may be controlled based at least in part on sensor measurements of the subject 512 by the glucose level sensor 310 without the glucose level sensor 310 communicating directly with the medicament delivery device 312. Advantageously, communication or control via the electronic device 252 enables the use of complex control algorithms or processes to control a medicament delivery device 312 (e.g., a patch pump) that may have limited or no computer processing capabilities.

### Example Controller for a Glucose Level Control System

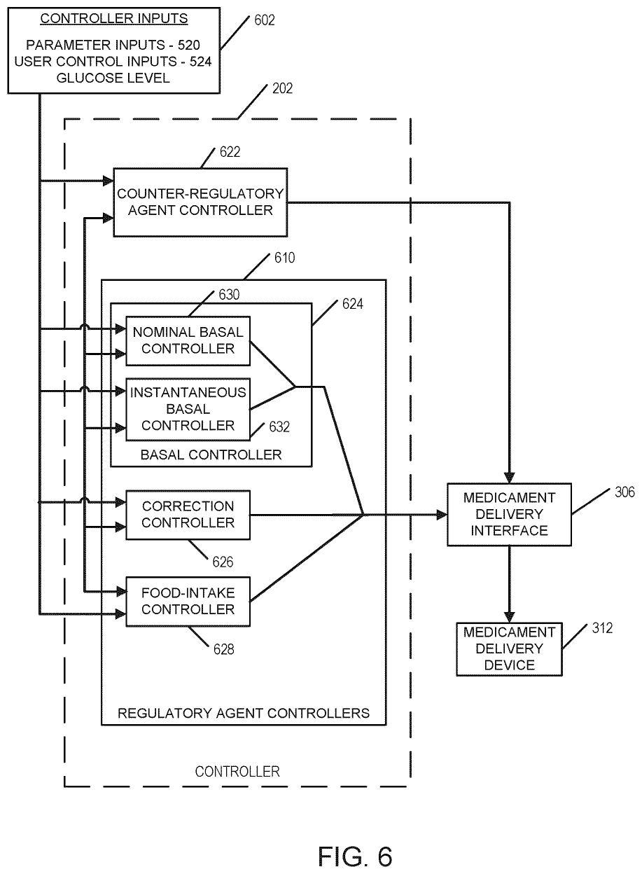

FIG. 6 illustrates a block diagram of an example controller 202 in accordance with certain embodiments. The controller 202 illustrated in FIG. 6 may represent a physical structure of different controllers or processors, or a logical structure that is implemented by one or more physical processors. In other words, a single processor may be used to implement each of the controllers illustrated in FIG. 6, each controller may be implemented by its own processor, or certain processors may implement multiple, but not necessarily all, of the controllers illustrated in FIG. 6 as part of the controller 202. Further, as illustrated in FIG. 6, each of the controllers may be implemented or part of the controller 202. Thus, to simplify discussion and not to limit the present disclosures, the counter-regulatory agent controller 622 and the regulatory agent controllers 610, including the nominal basal controller 630, the instantaneous basal controller 632, the correction controller 626, and the food-intake controller 628, may be referred to as sub-controllers or as sub-controllers of the controller 202. Moreover, although the controllers of FIG. 6 are illustrated as part of (e.g., as sub-controllers) the controller 202, in some implementations, one or more of the controllers may be separate from the controller 202. In cases where each of the sub-controllers are implemented as separate from the controller 202, the controller 202 itself may be omitted. In other words, each sub-controller may be implemented as an independent controller. Further, some or all of the controllers may be implemented by a processor 530, by the controller 202, or by a combination of the one or more processors 530 and the controller 202.

In the illustrated example, the controller 202 may include four separate controllers. However, it should be understood that more or fewer controllers are possible. In some cases, the controller 202 may be a bihormonal controller capable of controlling the administering of multiple hormones and/or medicaments. For example, some of the sub-controllers of the controller 202 may be regulatory agent controllers 610 while one or more of the controllers may include a counter-regulatory agent controller 622 (e.g., a glucagon controller). The regulatory agent controllers 610 may be configured to prevent or reduce the occurrence or risk of hyperglycemia by causing regulatory agent to be administered by the medicament delivery device 312. The regulatory agent controllers 610 may include a basal controller 624, a correction controller 626, and a food-intake controller 628.

The basal controller 624 may include a controller that can regulate basal insulin delivery. The basal insulin delivery may comprise a regular or periodic delivery of insulin that attempts to maintain a stable or steady glucose level of the subject 512. Basal insulin is often used to maintain the glucose level of the subject 512 outside periods of food-intake or increased activity times (e.g., exercise). Basal insulin may be delivered relatively frequently (e.g., once every five minutes, once an hour, etc.) or relatively less frequently (e.g., once a day, twice a day, etc.). The frequency with which basal insulin is delivered may be based at least in part on the type of insulin delivered. For example, long-acting insulin (LAI) may be delivered less frequently than fast-acting insulin (FAI).

The basal controller 624 may include a nominal basal controller 630 and an instantaneous basal controller 632. The nominal basal controller 630 may control administering of a basal dose over time based on a nominal basal rate. The nominal basal rate may be slowly adapted over time (e.g., on the order of a day). Further, the nominal basal rate may be based on a weight of the subject 512 and/or other subject-specific characteristics. In some cases, irrespective of basal rate adaption, the nominal basal rate may differ during different times of the day. For example, there may be one nominal basal rate during the day and another during the night. As another example, there may be a different nominal basal rate every 6 or 8 hours. Each of these nominal basal rates may be adapted over a longer time period (e.g., on the order of a day) enabling longer-term changes in a subject's insulin needs.

The instantaneous basal controller 632 may adjust the basal dose on a shorter time scale (e.g., every 5-minutes, on the order of an hour, etc.). The instantaneous basal controller 632 may adjust each basal dose at a discrete time interval (e.g., every 5-minutes or every hour). In some cases, the instantaneous basal controller 632 may adjust or adapt the basal dose centered around the nominal basal rate or a nominal basal dose determined based at least in part on the nominal basal rate.

The correction controller 626 may cause regulatory agent (e.g., insulin or insulin analog) to be administered to the subject 512 via the medicament delivery device 312 based at least in part on a glucose level or a change in glucose level of the subject 512. In other words, the correction controller 626 may cause medicament to be delivered to correct a divergence in a desired or target range (e.g., a setpoint range) of the glucose level of the subject 512. This medicament dose may be referred to as a correction dose. In some cases, the delivery of the correction dose may be triggered by the glucose level of the subject 512 exceeding a threshold value (e.g., an upper value of the target range). The correction dose may be selected to modify the glucose level of the subject 512 to be at or within a threshold distance of a setpoint target.

In some cases, the correction controller 626 may implement a model predictive control (MPC) algorithm and may be referred to as a model predictive controller. Alternatively, or in addition, the correction controller 626 may implement a biexponential pharmacokinetic (PK) model as described in some of the Controller Disclosures.

The food-intake controller 628 may cause regulatory agent (e.g., insulin or insulin analog) to be administered to the subject 512 via the medicament delivery device 312 based at least in part on a determination of a food-intake event. In some cases, the food-intake controller 628 may be or may be referred to as a priming insulin controller or a meal controller. The regulatory agent or medicament dose may be referred to as a food-intake dose. The food-intake dose may be a prandial insulin dose, a post-prandial insulin dose, or may be divided into both a prandial and post-prandial insulin dose.

A food-intake event may be associated with the consumption of food and may be determined to occur any time food is consumed. In other cases, a food-intake event may be limited to particular food consumption events, such as meals. Meals may include breakfast, lunch, or dinner, but is not limited to these particular meals. Food-intake events associated with meals may be referred to as meal-intake events. In yet other cases, food-intake events may be associated with the consumption of food of at least a minimum size or quantity of macronutrients. In other words, a snack below a threshold number of calories or carbohydrates may not qualify as a food-intake event while a snack exceeding the threshold number of calories of carbohydrates may quality as a food-intake event, regardless of whether the food-intake event is categorized as a meal.

The food-intake controller 628 may determine whether a food-intake event has occurred, or will occur, based at least in part on a food-intake announcement, which may also be referred to as a meal-announcement. A food-intake announcement may include an interaction by a user with a user interface of the glucose level control system 300 or a device in communication with the glucose level control system 300 that the subject 512 is consuming or will consume a meal. Further, the food-intake announcement may include a qualitative or quantitative indication of a size of the food-intake or of macronutrients of the food-intake. Alternatively, or in addition, the food-intake controller 628 may determine a food-intake event based at least in part on glucose levels of the subject 512.

In some embodiments, food-intake is managed by the correction controller 626. In such embodiments, the correction controller 626 may cause a correction dose of medicament to be administered in response to a food-intake event. Further, in some such embodiments, the food-intake controller 628 may be optional or omitted.

In some embodiments, each of the sub-controllers (e.g., one or more of the regulatory agent controllers 610 and/or the counter-regulatory agent controller 622) of the controller 202 may independently generate an output signal to cause the medicament delivery device 312 to administer medicament. Thus, each sub-controller may provide an output signal to the medicament delivery interface 306. However, as illustrated in FIG. 6, in some cases, two or more of the output signals from two or more of the sub-controllers may be combined to form a medicament (e.g., insulin) dose control signal that is based at least in part on the output of two or more of the two or more sub-controllers. For example, an output signal from the nominal basal controller 630 and an output signal from the instantaneous basal controller 632 may be combined to form a single basal control signal. The basal control signal may be provided to the medicament delivery interface 306, which may cause the medicament delivery device 312 to administer a basal dose of medicament (e.g., insulin or insulin analog) to the subject 512. As another example, the basal control signal may be combined with a correction dose control signal output by the correction controller 626 and/or a food-intake dose control signal output by the food-intake controller 628.

Combining two or more of the control signals may include performing a superposition operation, a merge or join process, and/or a signal aggregation process. Alternatively, or in addition, the controller 202 may receive an output from two or more of the sub-controllers (e.g., the regulatory agent controllers 610 and/or the counter-regulatory agent controller 622) and may generate a signal for output to the medicament delivery interface 306 that is based at least in part on the outputs of each sub-controller. In some cases, the controller 202 may weight the output of each sub-controller equally. In other cases, the controller may weight the output of at least one sub-controller differently from the output of at least one other sub-controller. For example, an output signal from the food-intake controller 628 may be weighted differently than an output signal from the correction controller 626. As another example, an output from the counter-regulatory agent controller 622 may be weighted differently from the output of one or more of the sub-controllers of the regulatory agent controllers 610. In some cases, the output of the counter-regulatory agent controller 622 may be maintained separately from or may supersede an output from the regulatory agent controllers 610.

As mentioned above, the regulatory agent controllers 610 may be configured to prevent or reduce the occurrence or risk of hyperglycemia. Similarly, the counter-regulatory agent controller 622 may be configured to prevent or reduce the occurrence or risk of hypoglycemia. The counter-regulatory agent controller 622 may generate a counter-regulatory agent dose control signal that can be provided to the medicament delivery device 312 to cause the delivery of the counter-regulatory agent (e.g., glucagon) by the medicament delivery device 312. Alternatively, or in addition, the counter-regulatory agent controller 622 may generate a recommendation for a dose of the counter-regulatory agent, which may be output on a display of the glucose level control system 300, an electronic device 252, or a remote computing system 254.

The medicament delivery device 312 may support the delivery of both regulatory and counter-regulatory agent. Alternatively, the medicament delivery device 312 may include two separate delivery devices with one configured to delivery regulatory agent and another configured to delivery counter-regulatory agent. In yet other cases, the medicament delivery device 312 may deliver regulatory agent, and the counter-regulatory agent controller 622 may cause output of a recommendation of counter-regulatory agent when appropriate based at least in part on the glucose level of the subject 512. In yet other embodiments, the medicament delivery device 312 may include two or more delivery devices configured to administer a regulatory agent. For example, the medicament delivery device 312 may include a delivery device configured to administer LAI and not delivery device configured to administer FAI. In yet other cases, the medicament delivery device 312 may be a single delivery device configured to delivery multiple types of regulatory and/or counter-regulatory agents.

The counter-regulatory agent controller 622 may determine to administer a counter-regulatory agent dose or that a counter-regulatory agent dose is recommended based at least in part on a glucose level of the subject 512. In some cases, the counter-regulatory agent dose may be referred to as a correction dose. Thus, in some cases, a correction dose may be a regulatory agent or a counter-regulatory agent. Moreover, in some cases, the operations of the counter-regulatory agent controller 622 may be performed by the correction controller 626 and the counter-regulatory agent controller 622 may be omitted.

As illustrated in FIG. 6, the controller 202 may receive a set of controller inputs 602. The controller inputs 602 may include any inputs that can facilitate the controller 202, or any of the sub-controllers, generating the output signals. For example, the controller inputs 602 may include the one or more parameter inputs 520, the user control inputs 524, a glucose level or glucose level signal received from a glucose level sensor 310, or any other input that can affect operation of the controller 202. Additional or alternative inputs can include inputs from one or more other physiological sensors, such as but not limited to a heart rate sensor, a motion sensor, or a ketone sensor. Further, the controller 202 may provide some or all of the controller inputs 602 to each of the sub-controllers. In some cases, some of the controller inputs 602 are provided to some of the sub-controllers, while other controller inputs 602 are provided to other sub-controllers of the controller 202. Additionally, as illustrated, in some cases one or more of the sub-controllers may communicate with one or more other sub-controllers of the controller 202. For example, the nominal basal controller 630 may communicate with the instantaneous basal controller 632, which may affect the output of one or more of the nominal basal controller 630 or the instantaneous basal controller 632. As another example, the food-intake controller 628 may communicate with the correction controller 626 and/or the counter-regulatory agent controller 622. Communication between the sub-controllers of the controller 202 may be referred to as inter-controller signals. The inter-controller signals may be used to enable two or more of the sub-controllers to work in conjunction with each other and/or to provide information that may be used for that sub-controller's control function.

The controller 202, or any of its sub-controllers (e.g., the regulatory agent controllers 610 and/or the counter-regulatory agent controller 622 may operate in either an online mode or in an offline mode. Further, the controller 202 or any of its sub-controllers may operate in an automated mode or in a manual mode. In the automated mode, the controller 202 may regulate a glucose level of the subject 512 using one or more control algorithms, such as those disclosed in the Controller Disclosures. Further, the controller 202 may operate using adaptive automated control, such as described in the Controller Disclosures. The control algorithms may use one or more glucose level signals received from the glucose level sensor 310 to determine medicament dosing. The controller 202 may execute control methods or algorithms that include control parameters that are mathematically combined with measured and/or predicted glucose level values to generate an output value that is converted (either directly or via additional conditioning) into one or more dose control signals. For example, the controller 202 may implement a generalized predictive control (GPC) method, as described in U.S. Pat. No. 7,806,854, that incorporates a variety of control parameters. The control algorithms may be generally adaptive. For example, the control parameters of the control algorithms may be dynamically adjusted during operation to reflect changing operating circumstances. Further, by monitoring its own operation, one or more of the control algorithms may implement a “learning” aspect that enables the one or more control algorithms to adjust their operation to be more specifically tailored to an individual subject 512, thereby enhancing the control algorithm's effectiveness and reducing or avoiding a need for additional input information about the subject 512, or other user. It should be noted that the one or more parameter inputs 520, and/or one or more user control inputs 524 may form part of the control parameters used by the control algorithms. Additional control parameters may exist as internal parameters according to the specifics of the control algorithm. Selected control parameters, including one or more parameter inputs 520, user control inputs 524, and/or the internal parameters, may be dynamically adjusted to realize the adaptation of the control algorithm.

In certain embodiments, the controller 202 or the sub-controllers thereof may learn from recent past periods of online operation and may use that learning during offline operation. For example, the Controller Disclosures describe several methods that are usable independently or together to operate in offline mode based at least in part on past operations during online mode. One example method automatically calculates the correct size of a correction bolus of insulin at a time of receiving an isolated glucose measurement based at least in part on past online operation. The correction bolus may be administered by the glucose level control system 300 in response to a user control input 524. A second example method automatically calculates the correct size of a meal bolus (or food-intake bolus) of insulin and administers it in response to a user control input 524. Both methods utilize information obtained during past periods of online operation to automatically calculate correct values, freeing the user of a need to make the calculation or provide a correction factor and improving the accuracy of the medicament dose determination.

It should be understood that the controller 202 or one or more of the sub-controllers of the controller 202 may implement one or more of the algorithms described in the Controller Disclosures. For example, in some cases, the controller 202 and/or one or more of the sub-controllers of the controller 202 may implement a model predictive control (MPC) algorithm as described in some of the Controller Disclosures. Alternatively, or in addition, the controller 202 and/or one or more of the sub-controllers of the controller 202 may implement a biexponential pharmacokinetic (PK) model as described in some of the Controller Disclosures. Further, although each of the sub-controllers included in the controller 202 are described as causing medicament (e.g., insulin, insulin analog, glucagon, etc.) to be administered, in some cases, the controller 202 or its sub-controllers may generate a recommendation of a medicament dose, which may be output on a display for presentation to a user or to another device (e.g., an electronic device 252 or a remote computing system 254).

### Smart Charging Station

An ambulatory medicament device may include a battery that powers operation of the device. The battery may be a rechargeable battery. This rechargeable battery may be charged by connecting the ambulatory medicament device to a power supply, such as a wall outlet that connects the ambulatory medicament device to the mains electricity supply of a building. Alternatively, or in addition, a battery of the ambulatory medicament device may be charged by physically or electrically connecting the ambulatory medicament device to a charging station. This charging station may be a smart charging station in that in addition to the charging the ambulatory medicament device, the charging station may facilitate communication of data between the ambulatory medicament device and an electronic system, such as a remote system. Moreover, the charging station may include processing capabilities that can identify data, data destinations (e.g., the remote system), and/or system upgrades for the ambulatory medicament device, among other features.

FIG. 7A illustrates an example of a charging station 700 configured to charge and/or communicate with an ambulatory medicament device 100 in accordance with certain embodiments. The ambulatory medicament device 100 may be or may include a medicament pump. Alternatively, the ambulatory medicament device 100 may be an ambulatory medical device. The charging station 700 may include an inductive or capacitive charging pad or cradle. Although illustrated as a cradle in which the ambulatory medicament device 100 may be cradled or otherwise held in place, it should be understood that the charging station 700 may have a different shape. For example, the charging station 700 may include a completely flat charging surface or may be shaped as a sleeve or jacket configured to accept the ambulatory medicament device 100 into an internal space created by the sleeve or jacket.

The charging station 700 may include a charging element 702 (e.g., an inductive or capacitive charging element) that can transfer power from a power supply (e.g., a mains power supply) to an ambulatory medicament device 100 when the ambulatory medicament device 100 is positioned to at least partially align a corresponding charging element of the ambulatory medicament device 100 with the charging element 702. For example, the charging element 702 may be an inductive pad that is configured to generate a magnetic field when a current is supplied to an inductive coil of the inductive pad. This magnetic field may cause a current to flow in a corresponding inductive coil of the ambulatory medicament device 100 charging a battery of the ambulatory medicament device 100. It should be understood that other wireless charging technology may be used to wirelessly charge a battery of the ambulatory medicament device 100 using the charging station 700. In some embodiments, the charging element 702 of the charging station 700 and the corresponding charging element of the ambulatory medicament device 100 may include a wireless charging interface that conforms to a Qi standard (from the Wireless Power Consortium). Alternatively, or in addition, the charging station 700 and the ambulatory medicament device 100 may support the Rezence standard (from the AirFuel Alliance), the Open Dots standard (from the Open Dots Alliance).

The battery of the ambulatory medicament device 100 may be an internal inductively chargeable battery. In some embodiments, the battery of the ambulatory medicament device 100 is replaceable, and in other embodiments, the battery is not replaceable. In some embodiments, the ambulatory medicament device 100 is water resistant (e.g., up to 5 m, 10 m, 20 m, 50 m, or ranges including and/or spanning the aforementioned values) or waterproof.

Although the charging station 700 is described as capable of wirelessly charging the ambulatory medicament device 100, in some cases, the charging station 700 may charge the battery of the ambulatory medicament device 100 via a wired connection, such as a universal serial bus (USB) connection.

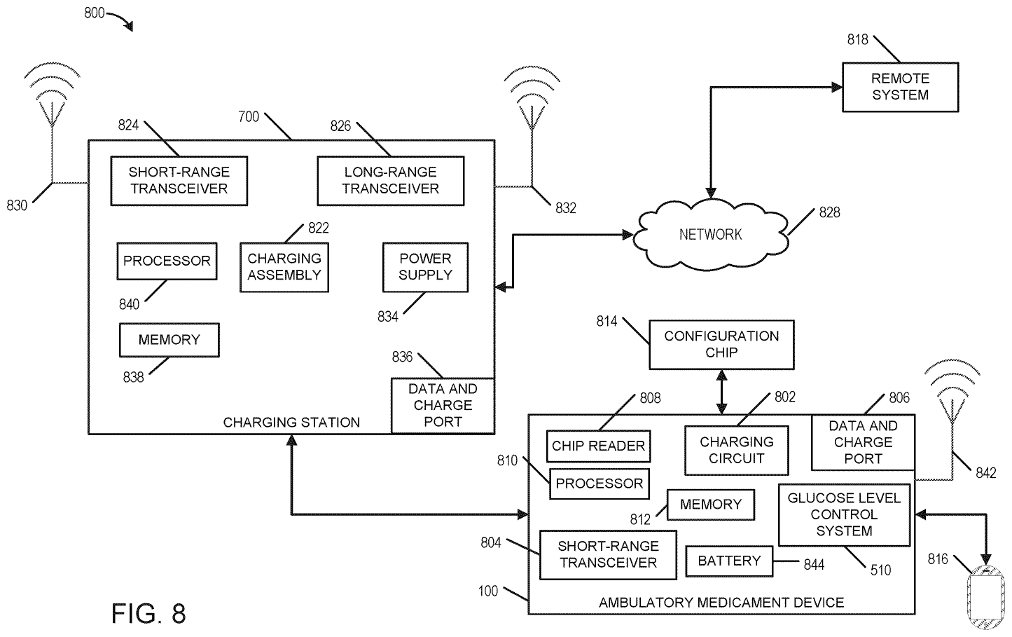

In addition to the charging element 702, the charging station 700 may include a communication system 704. The communication system 704 may include an antenna and a transceiver for wirelessly communicating with the ambulatory medicament device 100. Additional details of the communication system 704 are described below with respect to FIG. 8.

In some embodiments, recharging the ambulatory medicament device 100 may include one or more of the following operations: connecting the charging station 700 to a wall power outlet using a wired connection (e.g., a USB cable and plug); positioning the ambulatory medicament device 100 onto the charging station 700, verifying via a user interface of the ambulatory medicament device 100 and/or the charging station 700 that that the battery of the ambulatory medicament device is charging; and charging the ambulatory medicament device 100 via the charging station 700.

FIG. 7B illustrates an example of the ambulatory medicament device 100 being positioned on the charging station 700 of FIG. 7A in accordance with certain embodiments. As illustrated, the ambulatory medicament device 100 may be positioned within the charging station 700 to align a charging element (not shown) of the ambulatory medicament device 100 with charging element 702 of the charging station 700. Further, aligning the charging element of the ambulatory medicament device 100 with the charging element 702 of the charging station 700 may cause a communication system (not shown) of the ambulatory medicament device 100 to be aligned with the communication system 704 of the charging station 700 enabling communication between the ambulatory medicament device 100 and the charging station 700.

FIG. 7C illustrates an example of the ambulatory medicament device 100 being charged by the charging station 700 of FIG. 7A in accordance with certain embodiments. As illustrated, when placed on the charging station 700, a user interface (e.g., a touchscreen) of the ambulatory medicament device 100 may turn on and display an indication that the ambulatory medicament device 100 is charging and/or the battery charge level. In some embodiments, a light on the charging station 700 may illuminate or turn on while the ambulatory medicament device 100 is charging. When the charging is complete, the light may turn off or blink when it detects the presence of a ambulatory medicament device 100 that is fully charged or not charging. Alternatively, the light may blink when the ambulatory medicament device 100 is not charging or is not fully charged. It should be understood that the use of the light and the light status is just one example of a user interface to inform a user of the charging state of the ambulatory medicament device 100, and other user interfaces or user interface elements are possible.

In some embodiments, if the ambulatory medicament device 100 is not charging, the user can verify that the charging element 702 of the charging station 700 is properly aligned with the charging element of the ambulatory medicament device 100. In some embodiments, the typical time to fully charge a depleted battery may be equal to or less than about 2 hours, 4 hours, or ranges including and/or spanning the aforementioned values.

FIG. 7D illustrates an example of data communication between the ambulatory medicament device and the charging station of FIG. 7A in accordance with certain embodiments. As illustrated, when the ambulatory medicament device 100 is docked with or otherwise connected to the charging station 700, the ambulatory medicament device 100 may transfer data to the charging station 700 or vice versa. As indicated in FIG. 7D, the ambulatory medicament device 100 may display progress of the data transfer. In some cases, the ambulatory medicament device 100 may also output for display an indication of the data being transferred.

FIG. 8 illustrates a block diagram of a computing environment 800 that includes an ambulatory medicament device 100 interacting with a configuration chip 814 and/or a charging station 700 in accordance with certain embodiments. The ambulatory medicament device 100 may include one or more of the previously described embodiments. Further, the ambulatory medicament device 100 may include one or more of the systems illustrated and described with respect to the previous figures. For example, the ambulatory medicament device 100 may include the glucose level control system 510. Additionally, the ambulatory medicament device 100 may include a charging circuit 802, a short-range transceiver 804, a data and charge port 806, a chip reader 808, a processor 810, a memory 812, an antenna 842, and a battery 844.

The charging circuit 802 may include any circuit that can charge the battery 844. In some cases, the charging circuit 802 may include a charging element that can wirelessly receive power from a corresponding charging element 702 of the charging station 700. For example, the charging circuit 802 may include an inductive charging element configured to inductively receive power from the charging station 700. Alternatively, or in addition, the charging circuit 802 may receive power from a wired power connection, such as from the data and charge port 806. Further, the charging circuit 802 may include any kind of circuit that can convert the received power to a form useable to charge the battery 844. For example, the charging circuit 802 may include an AC-to-DC converter configured to convert AC power received from a mains power supply to DC power useable to charge the battery 844. In some cases, the charging station 700 converts the AC power to DC rendering the AC-to-DC converter of the ambulatory medicament device 100 unnecessary or optional.

The short-range transceiver 804 may include any type of transceiver that can send data to or receive data from a charging station 700. The short-range transceiver 804 may implement any type of communication protocol for near-distance or short-distance communication. For example, the short-range transceiver 804 may be a near-field transceiver that implements a near-field communications (NFC) protocol. Alternatively, or in addition, the short-range transceiver 804 may implement a Bluetooth® protocol, a Zigbee protocol, a Z-wave protocol, a Wi-Fi HaLow protocol (also known as IEEE 802.11 ah), or any type of custom short-range protocol. The short-range transceiver 804 may communicate using an antenna 842 that is configured to operate in one or more frequency ranges supported by the short-range transceiver 804. Generally, the short-range transceiver 804 and antenna 842 enable the ambulatory medicament device 100 to communicate with devices that support the same communication protocols as the ambulatory medicament device 100 and are located relatively close to the ambulatory medicament device 100. For example, the communication distance of the ambulatory medicament device 100 may be within ten meters or less, such as with Bluetooth®. In some cases, the communication distance may be shorter, such as 20 cm or less as with NFC. The combination of the short-range transceiver 804 and the antenna 842 may be used to communicate ambulatory medicament device data to the charging station 700 and/or to receive data or software from the charging station 700.

Alternatively, or in addition, the ambulatory medicament device 100 may receive power to charge the battery 844 and/or communicate data with the charging station 700 via the data and charge port 806. The data and charge port 806 may include any type of port that can be used to receive power for charging the battery 844. Further, the data and charge port 806 may include any type of port that can both receive and send data, such as ambulatory medicament device data. For example, the data and charge port 806 may include a universal serial bus (USB) plug or a universal serial bus (USB) port. In some such cases, the short-range transceiver 804 may be a universal serial bus (USB) transceiver that can communicate with the data and charge port 806.

The chip reader 808 may include any type of system that can access data from a configuration chip 814. In some cases, the chip reader 808 may emit electromagnetic energy that can be used to power a passive or semi-passive configuration chip 814. When the configuration chip 814 is brought within a particular distance of the chip reader 808 (e.g., within a few centimeters or less), the electromagnetic energy emitted by the chip reader 808 may provide enough power to cause the configuration chip 814 to transmit data to the chip reader 808. In some cases, the chip reader 808 may be a near-field communication (NFC) chip reader configured to read radio frequency identification (RFID) tags, terahertz frequency identification (TFID) tags, or other NFC tags. Moreover, the configuration chip 814 may be an RFID tag, a TFID tag, or any other type of NFC readable tag. Further, the configuration chip 814 may include any type of chip or tag capable of storing configuration information for the ambulatory medicament device 100. This configuration information may include initial settings for control parameters of a control algorithm used by the glucose control system 510. Alternatively, or in addition, the configuration information may include any information used to configure the ambulatory medicament device 100, such as when to trigger an alarm, what data to log, or what application features to enable, etc.

In some cases, the chip reader 808 may be included as part of the short-range transceiver 804. In other cases, the chip reader 808 may operate in conjunction with the short-range transceiver 804 to enable the ambulatory medicament device 100 to receive or access data from the configuration chip 814.

In some embodiments, the configuration information or data may be obtained from an electronic device other than the configuration chip 814. For example, the configuration information may be provided by a smartphone 816 or other electronic device that may support communication with the short-range transceiver 804 of the ambulatory medicament device 100. In some cases, the configuration chip 814 may be included as part of another electronic device, such as the smartphone 816, a tablet, a smartwatch, smart glasses, a key fob, a portable memory device, or any other type of electronic device that may store information for configuring the ambulatory medicament device 100. Moreover, in some cases, the configuration chip 814 may be included as part of another ambulatory medicament device. In other words, in some cases, an ambulatory medicament device may be used to initially configure, or to modify a configuration, or another ambulatory medicament device 100. Advantageously, using one ambulatory medicament device to configure another ambulatory medicament device may enable the transfer of configuration data between ambulatory medicament devices to personalize a replacement ambulatory medicament device.

The processor 810 can include any type of processor that may operate the ambulatory medicament device 100 by, for example, executing computer-readable instructions stored in the memory 812. The processor 810 may be a general-purpose processor or a special purpose processor specifically designed to operate the ambulatory medicament device 100. Further, the processor 810 may be configured to operate one or more of the chip reader 808, the glucose control system 510, the data and charge port 806, the charging circuit 802, the short-range transceiver 804, or other systems of the ambulatory medicament device 100. In some cases, the processor 810 operates in place of the processor 530 or is replaced by the processor 530.

The memory 812 may include any type of memory capable of storing configuration information for the ambulatory medicament device 100. Further, the memory 812 may store ambulatory medicament device data. Moreover, the memory 812 may include computer-executable instructions capable of being executed by the processor 810 and/or the processor 530. In some cases, the memory 812 operates in place of the memory 540 or is replaced by the memory 540.

The battery 844 may include any type of battery that can power the ambulatory medicament device 100. The battery 844 may be a rechargeable battery capable of being recharged by the charging station 700.

The charging station 700 can include any power supply or charger that can charge the battery 844 of the ambulatory medicament device 100 via a wired or wireless (e.g., inductive or capacitive) connection. The charging station 700 may be connected to a mains plug to obtain an AC power signal from a main electricity supply of a building. The charging station 700 may then use the AC power signal to charge the battery 844 of the ambulatory medicament device 100. The charging station 700 may include a short-range transceiver 824, an antenna 830, a long-range transceiver 826, an antenna 832, a charging assembly 822, a power supply 834, a processor 840, a memory 838, and a data and charge port 836.

The charging assembly 822 may include any charging circuit capable of transferring power to the charging circuit 802 for charging the battery 844. For example, the charging assembly 822 may include a wireless charging circuit or a wireless charging assembly. This wireless charging assembly may include an inductive charging circuit, which may include an inductive coil that can generate an electromagnetic field in response to a received current, which may in turn cause a corresponding inductive coil of the charging circuit 802 to generate a current that may be used to charge the battery 844. As another example, the charging assembly 822 may include a capacitive plate the pairs with a corresponding capacitive plate of the charging circuit 802. The alignment of the capacitive plates may generate a voltage that can charge the battery 844. The charging assembly 822 may include one or more of the embodiments previously described with respect to the charging circuit 802. Further, the charging assembly 822 may include any type of wireless charging protocol such as Qi, the Rezence standard, or the Open Dots standard, etc.

The short-range transceiver 824 may include any type of short-range transceiver that can implement at least some of the same near-field or short-range communications protocol as the short-range transceiver 804 of the ambulatory medicament device 100. In some cases, the short-range transceiver 824 may implement additional or other near-field or short-range communications protocols enabling the charging station 700 to support communication with different ambulatory medicament devices. Some non-limiting examples of communication protocols that may be supported by the short-range transceiver 824 include near-field communication, Wi-Fi HaLow (also known as IEEE 802.11ah), Zigbee, Z-wave, Bluetooth®, Bluetooth Low Energy (BLE), etc. Further, the short-range transceiver 824 may include one or more of the embodiments described with respect to the short-range transceiver 804. The short-range transceiver 824 in conjunction with the antenna 830 can communicate data with the ambulatory medicament device 100. The antenna 830 may be configured to support the same transmission frequencies as the antenna 842. The short-range transceiver 824 may be configured to receive ambulatory medicament device data from the short-range transceiver 804 of the ambulatory medicament device 100. Further the short-range transceiver 824 may be configured to transmit data (e.g., updated control parameters, software updates, etc.) to the ambulatory medicament device 100.

The long-range transceiver 826 may include any type of transceiver capable of communicating with a wide-area network, a cellular network, or any other type of network that includes communication with network or computing systems that are typically more than a few meters away from the long-range transceiver 826. In some cases, the long-range transceiver 826 may communicate with electronic devices over the Internet. The long-range transceiver 826 in conjunction with the antenna 832 may communicate with a remote system 818 over a network 828. The long-range transceiver 826 may support communication protocols capable of transmitting over a longer distance than communication protocols supported by the short-range transceiver 824 and/or the short-range transceiver 804. For instance, the long-range transceiver 824 may be a wide-area network transceiver or a cellular network transceiver. Thus, while the ambulatory medicament device 100 may communicate ambulatory medicament device data, using the short-range transceiver 804, when positioned within a range supported by a short-range or near-field protocol (e.g., 10 meters or less, 3 centimeters or less, etc.), the charging station 700 can communicate the ambulatory medicament device data, using the long-range transceiver 826 over ranges supported by wide-area communication or cellular communication protocols (e.g., WiFi, 4G LTE, 5G, etc.).

Generally, the short-range transceiver 824 and the long-range transceiver 826 support different communication protocols. However, in some cases, the short-range transceiver 824 and the long-range transceiver 826 may support at least some of the same communication protocols. In some embodiments, the short-range transceiver 824 and the long-range transceiver 826 may be combined. In such cases, the combined transceiver may support both short-range communications with the ambulatory medicament device 100 and long-range communications with the remote system 818. Similarly, the antenna 830 and the antenna 832 may be combined with the combined antenna supporting frequencies supported by the short-range transceiver 824, the long-range transceiver 826, or the combined transceiver. In some cases, the ambulatory medicament device 100 may communicate with the charging station 700 using the same protocols and/or communication technology that the charging station 700 uses to communicate with the network 828. In such cases, the charging station 700 may include a single transceiver (e.g., the long-range transceiver 826).

The data and charge port 836 may be configured to establish a charge and/or data connection with the corresponding data and charge port 806 of the ambulatory medicament device 100. The data and charge port 836 may include a universal serial bus plug or a universal serial bus port. In some such cases, the short-range transceiver 824 may be a universal serial bus transceiver that is in communication with the data and charge port 836. The charge and/or data connection may be established by connecting a physical wire or cable (e.g., a USB cable) between the data and charge port 836 and the data and charge port 806. Thus, in some embodiments, the ambulatory medicament device data may be communicated over a wired connection between the charging station 700 and the ambulatory medicament device 100. The ambulatory medicament device data may be communicated over the network 828 to the remote system 818 using a wireless connection (e.g., using the long-range transceiver 826) or a wired connection to a network device (e.g., a router).

Whether using a wired or wireless connection, the connection established between the charging station 700 and the ambulatory medicament device 100 may be a secure connection. The secure connection may be established using asymmetric encryption or any other type of encryption. In some cases, the charging station 700 and the ambulatory medicament device 100 may exchange public keys enabling the asymmetric encryption. Further, the charging station 700 and the ambulatory medicament device 100 may use encryption keys to generate a shared secret that may be used to secure traffic between the ambulatory medicament device 100 and the charging station 700. In some cases, the ambulatory medicament device 100 may encrypt the ambulatory medicament device data such that the remote system 818 may decrypt the data, but the charging station 700 may not be able to decrypt the data.

The power supply 834 may include any circuitry that can receive power from a wall outlet or other power source to power the charging station 700. Further, the power received by the power supply 834 may be supplied to the charging assembly 822 to charge the ambulatory medicament device 100. The power supply 834 may include circuitry that converts the received power into a form useable for charging the ambulatory medicament device 100. For example, the power supply 834 may up or down convert the voltage. As another example, the power supply 834 may convert the power from AC to DC.

The processor 840 can include any type of processor that may operate the charging station 700, for example, executing computer-readable instructions stored in the memory 838. The processor 840 may be a general-purpose processor or a special purpose processor specifically designed to operate the charging station 700. Further, the processor 840 may be configured to operate one or more of the short-range transceiver 824, the long-range transceiver 826, the data and charge port 836, the charging assembly 822, or other systems of the charging station 700. In some cases, the processor 840 includes one or more of the embodiments described with respect to the processor 810.

The memory 838 may include any type of memory capable of storing configuration information for the charging station 700. Further, the memory 838 may store ambulatory medicament device data, software or applications (e.g., upgrades, patches, etc.) to be transmitted to the ambulatory medicament device 100, configuration data for the ambulatory medicament device 100, or any other type of data that may be communicated to the ambulatory medicament device 100. In addition, the memory 838 may store any data received from the ambulatory medicament device 100 that is to be transmitted to a remote system 818. For example, the memory 838 may receive ambulatory medicament device data from the ambulatory medicament device 100 and transmit it to the remote system 818 (e.g., a server of a healthcare provider). Moreover, the memory 838 may include computer-executable instructions capable of being executed by the processor 840. In some cases, the memory 838 may include one or more of the embodiments described with respect to the memory 812.

In some implementations, the memory 838 may store ambulatory medicament device data, or other data, at least until a connection to the remote system 818 is available. Once the connection is available (and in some cases, once authorization/authentication is established), the ambulatory medicament device data, or other data, may be transmitted to the remote system 818. The data may be stored indefinitely or until a command is received. Alternatively, once the data has been transmitted, it may be deleted from the memory 838 and the charging station 700. In other cases, the data may remain for a period of time, such as until the memory 838 is overwritten, until a fixed time period has passed, or until a delete command is received, etc.

The memory 838 may store data to be transmitted to the ambulatory medicament device 100, such as software patches, feature upgrades, user access control information, or configuration data. The memory 838 may store the data at least until a connection to the ambulatory medicament device 100 is available (and in some cases, until authorization/authentication is established). As with the data to be transmitted to the remote system 818, the memory 838 may delete the data after transmission or may store the data to be transmitted to the ambulatory medicament device 100 indefinitely or for a fixed period of time as described above. As described herein, the data may include any type of data. For example, the data may include an application update to an application executing on the ambulatory medicament device 100; an updated control parameter or control parameter value for the ambulatory medicament device 100; an access permissions update to access permissions of a user of the ambulatory medicament device 100; medicament device data from a second ambulatory medicament device (e.g., to enable replacement of the an ambulatory medicament device while maintaining at least some subject-specific data); or any other type of data that may be transmitted to or from the ambulatory medicament device 100.

The memory 838 may include any type of memory. For example, the memory 838 may include secondary or long-term memory (e.g., a hard disk, a flash drive, solid state drive, etc.), primary short-term memory (e.g., RAM, etc.), virtual memory, cache, or any other type of memory. In some cases, the memory 838 may serve as a buffer to at least temporarily store the data as it is being received from the ambulatory medicament device 100 or the remote system 818, or as it is being transmitted to the ambulatory medicament device 100 or the remote system 818.

As described above, the charging station 700 may not only charge the ambulatory medicament device 100 but may also be configured to facilitate communication between an ambulatory medicament device 100 and a remote system 818. Advantageously, the charging station 700 enables an ambulatory medicament device 100 that may not be capable of directly communicating over a wide area network to transmit data to a system that is beyond the communication range of the short-range transceiver 804.

The remote system 818 may include any system that may be configured to receive from or provide data to an ambulatory medicament device 100. For example, the remote system 818 may be a cloud or network storage provider system, a healthcare server of a healthcare provider, or any other system capable of receiving, storing, logging, or processing ambulatory medicament device data from one or more ambulatory medicament devices 100. As another example, the remote system 818 may include any system of a healthcare provider or ambulatory medicament device 100 manufacturer that may make available updates, patches, or other new or different software features or versions of software used to control the ambulatory medicament device 100 or otherwise provide features of the ambulatory medicament device 100 to a subject or user.

### Example Charging Station Based Ambulatory Medicament Device Data Transfer Process

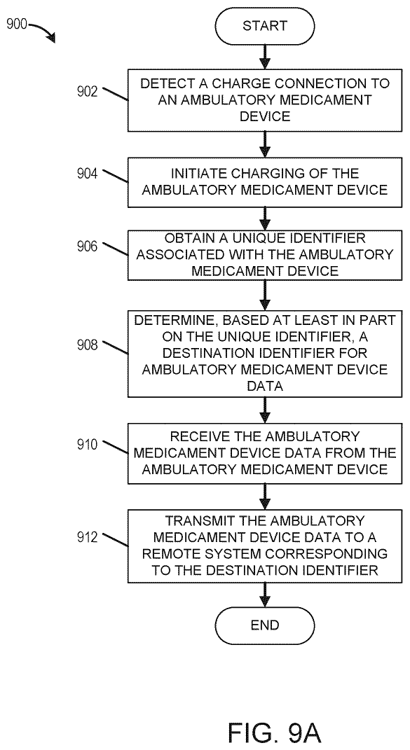

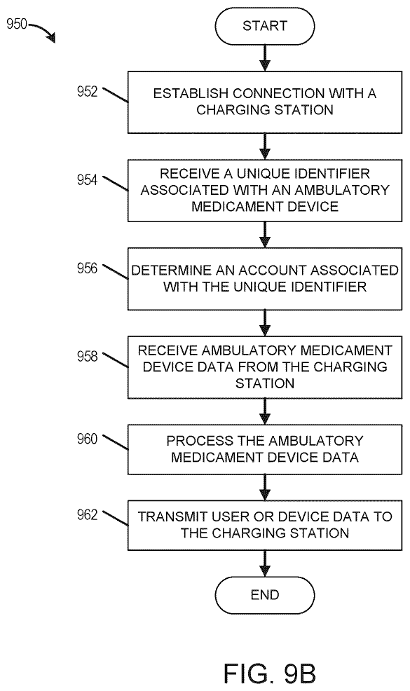

FIG. 9A illustrates a flowchart of an example charging station based ambulatory medicament device data transfer process 900 in accordance with certain embodiments. The process 900 can be implemented by any system that can charge an ambulatory medicament device 100 and that can communicate ambulatory medicament device data between the ambulatory medicament device 100 and a remote system 818. The process 900, in whole or in part, can be implemented by, for example, a charging station 700, a processor 840, a charging assembly 822, a short-range transceiver 824, a long-range transceiver 826, or a data and charge port 836, among others. Although any number of systems, in whole or in part, can implement the process 900, to simplify discussion, the process 900 will be described with respect to particular systems.

The process may begin at block 902 where, for example, the processor 840 using, for example, the charging assembly 822 or the data and charge port 836, detects a charge connection to an ambulatory medicament device 100. This charge connection may be a wired or wireless charging connection. For example, the charge connection may be via the data and charge port 836, which may include a USB port or other physical port. As another example, the charge connection may be via an inductive or capacitive charge connection. The charge connection may include any type of wireless charging standard including, but not limited to, the Wireless Power Consortium's Qi standard or the Power Matter Alliance's PMA standard.

Detecting the charge connection may include any process for determining that an electrical connection with the ambulatory medicament device 100 has been established. In some cases, the block 902 may include receiving a status of the battery 844 and/or a request to charge the battery 844 from the charging station 700. Further, detecting the charge connection may include determining that a circuit has been formed or completed between the charging assembly 822 and the charging circuit 802. For example, the block 902 may include determining that an inductor coil of the charging circuit 802 has been aligned, or sufficiently aligned, with a corresponding inductor coil of the charging assembly 822 enabling the charging assembly 822 to cause a current to flow in the charging circuit 802. Alternatively, the block 902 may include determining that a signal has been generated by a sensor (not shown) of the charging station 700 indicating sufficient proximity of the ambulatory medicament device 100 to charge the battery 844 of the ambulatory medicament device 100. In some cases, the block 902 may include detecting activation or interaction with a user interface element. For example, detecting the charge connection may include detecting that a button has been pressed by placement of the ambulatory medicament device 100 and/or by a user.

At block 904, the charging assembly 822 initiates charging of the battery 844 of the ambulatory medicament device 100. Initiating charging of the battery 844 may include permitting electricity to flow between the charging station 700 and the ambulatory medicament device 100. Permitting electricity to flow may include the charging assembly 822 causing a current to flow in the charging circuit 802 of the ambulatory medicament device 100. In some cases, an indication of the charge status of the battery 844 may be received by the charging station 700. If it is determined that the battery 844 is charged to a particular threshold (e.g., fully charged), the charging station 700 may cease charging the ambulatory medicament device 100. Alternatively, the charging station 700 may permit a trickle charge to flow between the charging station 700 and the ambulatory medicament device 100 to maintain the charge level of the battery 844 until the ambulatory medicament device 100 is removed from the charging station 700 without over-charging the battery 844. As described above, the battery 844 may be wirelessly charged by completing a circuit between the charging assembly 822 and the charging circuit 802. However, in some cases, wired charging may occur by connecting the data and charge port 836 and the data and charge port 806 using a charge cable (e.g., a USB cable).

At block 906, the processor 840 obtains a unique identifier associated with the ambulatory medicament device 100. The unique identifier may include any information that uniquely identifies the ambulatory medicament device 100 and/or a subject that uses the ambulatory medicament device 100. For example, the unique identifier may be a serial number or a network communication identifier (e.g., a media access control (MAC) address) of the ambulatory medicament device. As another example, the unique identifier may be a subject identifier (e.g., social security number, unique username, etc.) associated with the subject that receives medicament therapy from the ambulatory medicament device 100.

In certain embodiments, the processor 840 may register the ambulatory medicament device 100 with the charging station 700 based on the obtained unique identifier. Registering the ambulatory medicament device 100 may include receiving subject identifying information from a subject that uses the ambulatory medicament device 100. Further, registering the ambulatory medicament device 100 may include receiving permissions associated with ambulatory medicament device 100 or the subject associated with the ambulatory medicament device 100. For example, the subject may indicate whether the charging station 700 is permitted to receive ambulatory medicament device data, or the type of ambulatory medicament device data the charging station 700 is permitted to receive from the ambulatory medicament device 100 when in communication with the charging station 700. In some cases, registering the ambulatory medicament device 100 may include accessing an electronic device of a user (e.g., the subject or a guardian of the subject) to confirm that the charging station 700 is authorized to access ambulatory medicament device data. Further, registering the ambulatory medicament device 100 may include providing the charging station 700 with account information or access to the remote system 818 or an account of a subject at the remote system 818.

In some cases, the charging station 700 establishes a data connection with the ambulatory medicament device 100. This data connection may be a wireless connection between the short-range transceiver 824 and the short-range transceiver 804. Alternatively, the data connection may be a wired connection via a wire or cable (e.g., USB cable) connecting the data and charge port 836 to the data and charge port 806.

In some cases, the unique identifier is received upon establishing the data connection. Alternatively, or in addition, the unique identifier may be received as part of the data connection process. For example, the charging station 700 may receive the unique identifier as part of a communication handshake process with the ambulatory medicament device 100. The charging station 700 may use the unique identifier received from the ambulatory medicament device 100 to determine whether to permit a data connection to the ambulatory medicament device 100. Alternatively, or in addition, the charging station 700 may provide a unique identifier of the charging station 700 to the ambulatory medicament device 100 enabling the ambulatory medicament device 100 to determine whether to permit a data connection.

At block 908, the processor 840 determines, based at least in part on the unique identifier, a destination identifier for the ambulatory medicament device data. The destination identifier may include an identification of the remote system 818. Alternatively, or in addition, the destination identifier may include an identification of an account at the remote system 818. The account may be associated with a subject that receives medicament therapy from the ambulatory medicament device 100. Alternatively, or in addition, the account may be associated with a user (e.g., parent, guardian, healthcare provider, etc.) that helps care for the subject.

In some cases, the destination identifier is determined by accessing data stored at the memory 838 that is associated with the unique identifier. For example, if the ambulatory medicament device 100 has been previously registered with the charging station 700, the charging station 700 may identify account information for accessing an account at the remote system 818 based on stored account information at the memory 838 that is associated with the unique identifier or the registered ambulatory medicament device 100.

Alternatively, or in addition, the destination identifier may be received from the ambulatory medicament device 100 with the unique identifier or in place of the unique identifier. In some such cases, the operations associated with the block 908 may be optional or omitted. Further, in some such cases, the destination identifier may be unnecessary because, for example, data packets received from the ambulatory medicament device 100 include an address of the remote system 818 as a destination for the data packets. Moreover, the destination packets may include account information for the subject. In other words, in some cases, the charging station 700 may serve as a router or pass-through device that directs data packets received from the ambulatory medicament device 100 without accessing the data stored in the data packets or without accessing more data than necessary to route the data packets to remote system 818.

In some cases, the operations associated with the blocks 906 and/or 908 may be optional or omitted. For example, in some cases, a destination (e.g., a remote system 818) for the ambulatory medicament device data, or other data, may be selected by a user, previously determined (e.g., during a registration or other configuration process), or predefined by a provider or manufacturer of the charging station 700. Further, in some cases, the ambulatory medicament device data may be sanitized of user-identifying information. In some such cases, the data may be transmitted to a remote system 818 whose access information (e.g., IP address, etc.) is stored at or otherwise determined by the charging station 700 without determining an identifier and/or unique identifier.

At block 910, the processor 840 receives the ambulatory medicament device data from the ambulatory medicament device. The block 910 may include using the short-range transceiver 824 to receive the ambulatory medicament device data via the antenna 830.

The ambulatory medicament device data may include any type of data that can be measured, detected, or predicted based on therapy provided by the ambulatory medicament device 100 and/or user-interaction with the ambulatory medicament device 100 or a user-interface of the ambulatory medicament device 100. For example, the ambulatory medicament device data may include clinical data corresponding to medicament therapy provided by the ambulatory medicament device 100 to the subject, such as an amount of medicament (e.g., insulin or counter-regulatory agent) administered, a timing of medicament administered, subject glucose level, an effect on glucose level of administered medicament, etc. As another example, the ambulatory medicament device data may include device data corresponding to the operation of the ambulatory medicament device 100, such as alarm data, medicament reservoir data, malfunction data, up-time data, data associated with access to a sensor (e.g., a continuous glucose monitor sensor), battery status, software version data, etc. As yet another example, the ambulatory medicament device data may include user interface data corresponding to user interaction with the ambulatory medicament device 100, such as meal announcements, responses to alarms, ambulatory medicament device 100 settings changes, data access times, therapy pauses (e.g., when removing for swimming), etc. Some types of data may be classified in different or multiple ambulatory medicament device data categories. For example, meal announcements may also be included as part of clinical data.

In some embodiments, the processor 840 may select ambulatory medicament device data to access and/or the processor 810 may select ambulatory medicament device data to transmit based at least in part on one or more of a plurality of data selection criteria. This data selection criteria may include any criteria for determining data to access or to provide to a remote system 818. For example, the data selection criteria may include: a determination that an application update for an application executing on the ambulatory medicament device 100 is available at the remote system 818. This application update may be a release of updated software or access to new features of the ambulatory medicament device 100. In some cases, the software update may be pre-downloaded to the charging station 700 for uploading to the ambulatory medicament device 100 upon connection by the ambulatory medicament device 100 with the charging station 700. In some cases, the determination that the application update is available may be based on accessing software version data from the ambulatory medicament device 100 or model data for the ambulatory medicament device 100.

As another example, the data selection criteria may include a determination that an updated control parameter having a control parameter value different from one stored on the ambulatory medicament device 100 is available. This new parameter value may be a value at or obtained from the remote system 818. Another data selection criteria may include a determination that access to an application feature of the application is permitted, such as due to the addition of a counter-regulatory agent cartridge to the ambulatory medicament device 100. In some cases, the application feature may be permission to access additional controls, such as long-acting insulin control. These additional controls may be granted in response to determining that the subject satisfies safe-access criteria. Examples of embodiments of an ambulatory medicament device 100 that may include safe access controls for permitting or restricting access to particular features of the ambulatory medicament device 100 that may be combined with features of the present disclosure are described in U.S. Provisional Application No. 63/169,112, and in International Application No. PCT/US21/72742, the disclosures of which are each hereby incorporated by reference in their entirety herein for all purposes.

Another example of data selection criteria may include a determination that an alarm has occurred at the ambulatory medicament device. The ambulatory medicament device data accessed may be any data associated with alarm, such as clinical data or device data recorded within a threshold time range of the alarm occurrence. Similarly, a determination that device data of the ambulatory medicament device 100 indicates occurrence of a malfunction at the ambulatory medicament device 100 may be used as data selection criteria to determine data to access from the ambulatory medicament device 100.

In some cases, the data selection criteria may be based on a determination that a time period has elapsed, such as a quantity of time since the last access of medicament device data. In some cases, the data selection criteria may be based on a determination that a time (e.g., a current time) is a scheduled data transfer time. For example, the data may be selected to include all data between a prior scheduled data transfer time and a current scheduled data transfer time. The scheduled data transfer time may be any particular time and may be set once or repeated any number of times. For example, the scheduled data transfer time may be each day at midnight, every Saturday night, or any other desired time.

The data selection criteria may also include a determination that a request for the ambulatory medicament device data has been received. In some cases, the data selection criteria may include a determination that clinical data does not satisfy expected clinical data. The expected clinical data may be based on a control algorithm, demographics of the subject (e.g., gender or age), physiological characteristics of the subject (e.g., weight, pre or post puberty, comorbidities, etc.), or any other factor that may impact expected or predicted data relating to the management of the subject's disease. A determinization that measured clinical data (e.g., a measured glucose level) is more than a threshold different from expected clinical data (e.g., an expected glucose level) may be used as a data selection criterion for determining what ambulatory medicament device data, if any, to select for transmission to the remote system 818.

The data selection criteria may further include a determination that user interface data does not satisfy an expected user interaction profile. The user interaction profile may include any type of profile of user interactions with the ambulatory medicament device 100. For example, the user interaction profile may include a history of user interactions with the ambulatory medicament device 100 and/or a prediction of expected user interactions in particular circumstances based at least in part on the history of user interactions. For example, ambulatory medicament device data may be selected based on a determination that recent meal announcements do not reflect expected meal announcements for the subject. As another example of user interface data not satisfying an expected user interaction profile, ambulatory medicament device data may be selected based on a determination that user responses to output alarms do not match expected user interface data when the alarms occur.

In some cases, the data selection criteria may include a determination that a backup therapy protocol has been generated or modified. For example, a determination of an update to a backup therapy protocol may result in the backup therapy protocol being transmitted to the remote system 818. In some cases, the backup therapy protocol may be stored at the memory 838 of the charging station 700 enabling access by a local user via a user interface (not shown) of the charging station 700 or by another (e.g., a replacement) ambulatory medicament device.

As described in the preceding paragraphs, there are a number of different data selection criteria that may be used by the charging station 700 or the ambulatory medicament device 100 to identify what ambulatory medicament device data, if any, to select and to communicate from the ambulatory medicament device 100 to the charging station 700 to enable, in some cases, the transmission of the ambulatory medicament device data to the remote system 818.

At block 912, the processor 840 transmits the ambulatory medicament device data to a remote system 818 corresponding to the destination identifier. The block 912 may include using the long-range transceiver 826 to transmit the ambulatory medicament device data via the antenna 832.

In some embodiments, the block 912 may include encoding the ambulatory medicament device data to obtain encoded data before transmitting the ambulatory medicament device data. In such cases, the processor 840 may transmit the encoded ambulatory medicament device data to the remote system 818 corresponding to the destination identifier. Encoding the ambulatory medicament device data may include encapsulating the ambulatory medicament device data in one or more transmit packets addressed to the remote system 818. Encapsulating the data may further include breaking the data is particular sized data chunks and wrapping the data chunks with network address information used to direct transmission of the data packets. Further, encoding the ambulatory medicament device data may include formatting the ambulatory medicament device data that enables or optimizes transmission of the data over a wide area network or a cellular network. Formatting the ambulatory medicament device data may include dividing the data into particular sized data chunks. In some cases, encoding the ambulatory medicament device data may include encrypting the data.

In some embodiments, the block 912 may include using account access information associated with a subject that receives medicament therapy from the ambulatory medicament device 100. This account access information may be used to access an account of the subject at the remote system 818. At least some of the account access information may be received from the ambulatory medicament device 100 as part of the operations performed at one or more of the blocks 906-910. In some cases, the account access information may be accessed from the memory 838. For example, in cases where the account access information has been previously received as part of a registration process or during a prior performance of the process 900, the account access information may be accessed from the memory 838 using, for example, the unique identifier obtained at the block 906 to identify the account access information from the memory 838. In some cases, the account access information may be separately provided as part of a user interaction with the charging station 700, such as during an initial registration process of the ambulatory medicament device 100 with the charging station 700. The user interaction may be directly with a user interface of the charging station 700 or via a user interface of the ambulatory medicament device 100 or another electronic device (e.g., a smartphone) capable of communicating with the charging station 700 and/or the ambulatory medicament device 100. The account access information may include information that identifies the remote system 818, an account at the remote system 818, and/or one or more users permitted to access the ambulatory medicament device data at the remote system 818. Further, transmitting the ambulatory medicament device data may include associating the ambulatory medicament device data with an account at the remote system 818, such as an account associated with or reference by the account access information.

The process 900 may be performed each time the ambulatory medicament device 100 is positioned with respect to the charging station 700 in a manner that permits charging of the battery 844 of the ambulatory medicament device 100. Alternatively, or in addition, the process 900 may be performed at set times or after a particular amount of time has elapsed since prior performance of the process 900. For example, assuming the battery 844 requires charging once a week, the process 900 may be performed once a week when the ambulatory medicament device 100 is electrically connected to the charging station 700. Alternatively, although the ambulatory medicament device 100 may be charged once a week, the process 900 may be performed once a month. In some cases, at least some of the process 900 is performed each time a charge connection is detected, while some of the process 900 is performed at particular times. As another example, the ambulatory medicament device 100 may be charged each time it is connected to the charging station 700, but data transfer may occur only when the charging station 700 has a connection to the network 828. In some cases, ambulatory medicament device data may be received by the charging station 700 regardless of access to the network 828. In some such cases, the ambulatory medicament device data may be stored at the memory 838 until at least such time as when the charging station 700 can access the network 828. In other cases, the ambulatory medicament device data is deleted from the memory 838 after a set period of time regardless of whether it has been transmitted to the remote system 818.

In some cases, the charging station 700 requires permissions to access ambulatory medicament device data. Thus, a charging station 700 that has not been granted access to an ambulatory medicament device 100 may charge a battery of the ambulatory medicament device 100 but may not be permitted to access ambulatory medicament device data of the ambulatory medicament device 100. For example, at the block 906 the charging station 700 may receive a unique identifier of an ambulatory medicament device 100 and may determine based at least in part on the unique identifier that the ambulatory medicament device 100 is not registered with the charging station 700. The charging station 700 may continue to charge the ambulatory medicament device 100 but the remainder of the process 900 may be omitted. Alternatively, or in addition, a user may be prompted to register the ambulatory medicament device 100 with the charging station 700. If the user elects to register the ambulatory medicament device 100 with the charging station 700 and grant the charging station 700 permission to access ambulatory medicament device data, the remainder of the process 900 may proceed after registration of the ambulatory medicament device 100.

In some embodiments, a remote system 818 may perform a similar or corresponding process as the process 900 to transfer data or software updates to the ambulatory medicament device 100 via the charging station 700 or to establish a connection with the charging station 700 to receive data provided by the ambulatory medicament device 100. FIG. 9B illustrates one such non-limiting example process. FIG. 9B illustrates a flowchart of an example remote server based data transfer process 950 in accordance with certain embodiments. The process 950 can be implemented by any system that can communicate with a charging station 700 to obtain data from an ambulatory medicament device 100 and/or to provide data or software updates to the ambulatory medicament device 100. The process 950, in whole or in part, can be implemented by, for example, a remote system 818, a charging station 700, a processor 840, a charging assembly 822, a short-range transceiver 824, a long-range transceiver 826, or a data and charge port 836, among others. Although any number of systems, in whole or in part, can implement the process 950, to simplify discussion, the process 950 will be described with respect to particular systems.

The process 950 may begin at block 952 where, for example, the remote system 818 establishes a connection with a charging station 700. It should be understood that either the 818/or the charging station 700 may initiate establishment of the connection. Further, the type of connection is not limited and may be a wireless or wired connection. In some cases, the connection may be established through a network, such as a cellular network and/or the Internet.

At block 954, the remote system 818 receives a unique identifier associated with an ambulatory medicament device 100. In some cases, the unique identifier is associated with the subject or a user. The block 954 may include one or more of the embodiments previously described with respect to the block 906.

At block 956, the remote system 818 determines an account associated with the unique identifier. The account may be associated with the ambulatory medicament device 100, the subject, or a user (e.g., such as a parent, guardian, or healthcare provider). The block 956 may include one or more of the embodiments previously described with respect to the block 908.

As with the blocks 906 and 908, in some cases, the operations associated with the blocks 954 and/or 956 may be optional or omitted. For example, in some cases, ambulatory medicament device data is not associated with a particular subject or ambulatory medicament device 100 but is instead sanitized of identifying information and collected for research purposes. In some such cases, the operations associated with identifying the account may be omitted. As another example, a patch may be pushed out to all ambulatory medicament devices. In such cases, the identity of the ambulatory medicament device 100 may be unnecessary. Alternatively, it may be desirable to obtain the unique identifier of the ambulatory medicament device 100 to track which devices have been updated.

At block 958, the remote system 818 receives ambulatory medicament device data from the charging station 700. In some cases, an identity of an associated ambulatory medicament device 100 may be received with the data. In other cases, the identity of the ambulatory medicament device 100 may be excluded or already determined at the block 954. In some cases, the ambulatory medicament device data may be associated with a particular time period. For example, the data may be associated with a day, a week, or a time period occurring since a previous data transfer operation.

At block 960, the remote system 818 processes the ambulatory medicament device data. Processing the ambulatory medicament device data may include any type of process that may be performed with respect to ambulatory medicament device data. Some nonlimiting examples of operations that may be performed at the block 960 may include storing the ambulatory medicament device data, associating the ambulatory medicament device data with an account of the subject and/or a user (e.g., a healthcare provider), aggregating the ambulatory medicament device data with other data or the subject and/or with data of one or more other subjects, analyzing the data, generating an alarm based on the data, transmitting the data to a target destination, filtering or sanitizing the data, and the like. In some cases, the operations associated with the blocks 958 and/or 960 may be optional or omitted. For example, in some cases, the remote system 818 may not receive data, but may instead transmit data.

At block 962, the remote system 818 transmits user or device data to the charging station 700. The user data may include ambulatory medicament device data that can be used to configure an ambulatory medicament device 100. Advantageously, transmitting the ambulatory medicament device data to the charging station 700 enables the charging station 700 to configure an ambulatory medicament device 100. In some cases, the process 900, the process 950 and/or a combination of the process 900 and the process 950 can be used to obtain data from one ambulatory medicament device and use it to help configure another ambulatory medicament device. The device data can include configuration data for an ambulatory medicament device 100 and/or a control algorithm executed by the ambulatory medicament device 100. In some cases, the block 962 can be optional or omitted. For example, the remote system 818 may receive data but may not transmit data to the charging station 700.

### Example Automatic Ambulatory Medicament Device Data Selection and Transfer Process

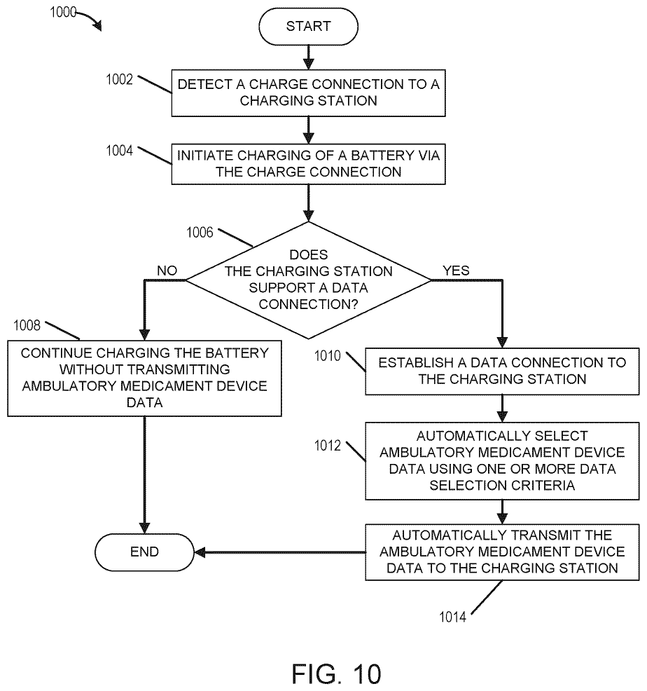

FIG. 10 illustrates a flowchart of an example automatic ambulatory medicament device data selection and transfer process 1000 in accordance with certain embodiments. The process 1000 can be implemented by any system that can automatically select ambulatory medicament device data and transfer a copy of the ambulatory medicament device data to a charging station 700 enabling the charging station 700 to provide the ambulatory medicament device data to a remote system 818. The process 1000, in whole or in part, can be implemented by, for example, an ambulatory medicament device 100, a processor 810, a charging circuit 802, a data and charge port 806, a glucose control system 510, or a short-range transceiver 804, among others. Although any number of systems, in whole or in part, can implement the process 1000, to simplify discussion, the process 1000 will be described with respect to particular systems.

The process may begin at block 1002 where, for example, the processor 810 using, for example, the charging circuit 802 or the data and charge port 806, detects a charge connection to a charging station 700. Detecting the charge connection may include any process for determining that an electrical connection with the charging station 700 has been established. In some cases, detecting the charge connection may include receiving power from the charging station 700. The power may be received in response to the ambulatory medicament device 100 transmitting a request for power to the charging station 700. Alternatively, or in addition, the block 1002 may include receiving a request for status of the battery 844 from the charging station 700. Advantageously, by requesting power transfer before charging the battery 844, overcharging can be prevented or reduced. The block 1002 may include one or more of the embodiments described with respect to the block 902.

At block 1004, the charging circuit 802 initiates charging of the battery 844 via the charge connection. Initiating charging of the battery 844 may include permitting electricity to flow between the charging station 700 and the ambulatory medicament device 100. Alternatively, or in addition, initiating charging of the battery 844 may include permitting received electricity to flow to the battery 844. Controlling the flow of electricity to the battery 844 may be used to prevent damage to the battery 844 due to overcharging. The block 1004 may include one or more of the embodiments previously described with respect to the block 904.

At decision block 1006, the processor 810 determines whether the charging station 700 supports a data connection. Determining whether charging station 700 supports a data connection may include determining whether the charging station 700 includes a transceiver (e.g., the short-range transceiver 824 and/or the long-range transceiver 826) or an antenna (e.g., the antenna 830 or the antenna 832). The determination of whether the charging station 700 supports a data connection may be based on whether the charging station 700 responds to a connection request from the ambulatory medicament device 100. Alternatively, or in addition, the processor 810 may determine that the charging station 700 supports a data connection in response to receipt of a connection request or other data from the charging station 700.

In some cases, determining whether the charging station 700 supports a data connection may include obtaining a unique identifier (e.g., serial number, MAC address, etc.) of the charging station 700. The processor 810 may determine based on the unique identifier of the charging station 700 whether the charging station 700 has permission or is authorized to access ambulatory medicament device data from the ambulatory medicament device 100 (e.g., whether the ambulatory medicament device 100 has been registered with the charging station 700 and/or vice versa). Determining whether the charging station 700 is authorized to access the ambulatory medicament device data and/or to establish a data connection may include determining whether the unique identifier is stored at the memory 812 of the ambulatory medicament device 100 or is associated with permissions at the memory 812 that are associated with permitting the data connection with the charging station 700 or the access of ambulatory medicament device data by the charging station 700. Thus, in some cases, the decision block 1006 may include determining whether the charging station 700 is capable of supporting a data connection and/or is permitted to establish a data connection with the ambulatory medicament device 100.

If it is determined at the decision block 1006 that the charging station 700 does not support a data connection, at block 1008, the charging circuit 802 continues charging the battery 844 without the processor 810 transmitting ambulatory medicament device data. Determining that the charging station 700 does not support a data connection may include determining that the charging station 700 is not capable of a data connection or is not permitted to establish a data connection with the ambulatory medicament device 100. Advantageously, by permitting charging regardless of data connection capabilities, an ambulatory medicament device of a user that is not registered with the charging station 700 may use the charging station 700 to charge its battery. Thus, for example, a guest can charge an ambulatory medicament device without potentially sensitive or private data being accessed from the ambulatory medicament device. Moreover, while the ambulatory medicament device 100 may not be registered with the charging station 700 and thus, not able to establish a data connection with the charging station 700, the ambulatory medicament device 100 may be registered with a different or a second charging station and may be able to transmit ambulatory medicament device data over a data connection established with the second charging station. In other words, the ambulatory medicament device of the guest may establish a data connection with the guest's charging station the next time the ambulatory medicament device is connection to the charging station owned by the guest.

If it is determined at the decision block 1006 that the charging station 700 does support a data connection, at block 1010, the processor 810 establishes a data connection to the charging station 700. Establishing the data connection may include registering with the charging station 700. Registering the ambulatory medicament device 100 with the charging station 700 may include providing the charging station 700 a unique identifier associated with the ambulatory medicament device 100 and/or a subject that receives therapy from the ambulatory medicament device 100. This unique identifier may be a serial number, a MAC address, or some other unique identifying number or alphanumeric value associated with the ambulatory medicament device 100. Alternatively, or in addition, the unique identifier may be a username, user identification number (e.g., a social security number), or other unique identifier of the subject, or other user associated with the subject. In some cases, registering the ambulatory medicament device 100 with the charging station 700 may include providing the charging station 700 with access information for accessing the remote system 818, or an account at the remote system 818, on behalf of a subject that receives therapy from the ambulatory medicament device 100. For example, registering the ambulatory medicament device 100 with the charging station 700 may include providing a username and password for an account of the subject at the remote system 818.

Alternatively, the ambulatory medicament device 100 may have been previously registered with the charging station 700. As described above, in some cases, registration with the charging station 700 may be a criterion for determining whether the charging station 700 supports a data connection with the ambulatory medicament device 100.

At block 1012, the processor 810 automatically selects ambulatory medicament device data using one or more data selection criteria. The ambulatory medicament device data may be selected without user interaction with the ambulatory medicament device 100. In some cases, the one or more data selection criteria may include at least some of the data selection criteria described with respect to the process 900. Moreover, the data selection criteria may include: a determination that an alarm has occurred at the ambulatory medicament device; a determination that device data of the ambulatory medicament device indicates occurrence of a malfunction at the ambulatory medicament device; a determination that a time period has elapsed; a determination of a time of a previous transmission of previous ambulatory medicament device data; a determination that a request for the ambulatory medicament device data has been received; a determination that clinical data does not satisfy expected clinical data; a determination that user interface data does not satisfy an expected user interaction profile; or a determination that a backup therapy protocol has been generated or modified.

In some cases, at least one of the data selection criteria may be based at least in part on a prior transmission time corresponding to a prior transmission of prior ambulatory medicament device data to the charging station 700, the remote system 818, or to another charging station. For example, the ambulatory medicament device data selected may be data that has been logged or generated since a prior transmission of the ambulatory medicament device data. The prior transmission time may be stored at the memory 812 enabling the ambulatory medicament device 100 to determine when data was last transmitted. Alternatively, or in addition, the ambulatory medicament device 100 may access the prior transmission time from the charging station 700. In yet other cases, the ambulatory medicament device 100 may send a query request to the charging station 700 to request that the charging station 700 query the remote system 818 to determine the prior transmission time. Advantageously, querying the remote system 818 for the prior transmission time enables the ambulatory medicament device 100 to determine whether a prior transmission was completed successfully, which may be used to select ambulatory medicament device data to include in a current transmission. In some cases, the block 1012 may include one or more of the embodiments described with respect to the block 910.

In some cases, at least one of the data selection criteria may be based at least in part on a determination of prior ambulatory medicament device data transmitted to the charging station 700 or to the remote system 818. For example, the ambulatory medicament device 100 may select ambulatory medicament device data by determining prior transmitted ambulatory medicament device data. The prior ambulatory medicament device data may be determined from a log or communication record stored, for example, at the memory 812. Alternatively, or in addition, the prior ambulatory medicament device data may be determined by transmitting a query to the charging station 700 to receive an indicator corresponding to at least some of the prior ambulatory medicament device data. This indicator may identify previously transmitted data and/or confirm successful transmission of previously transmitted data to the charging station 700 and/or the remote system 818.

At block 1014, the processor 810 automatically transmits the ambulatory medicament device data to the charging station 700. The transmission of the ambulatory medicament device data may occur without user interaction with the ambulatory medicament device 100. Transmitting the ambulatory medicament device data to the charging station 700 may include transmitting a destination identifier corresponding to the remote system 818 to the charging station 700. In some cases, the destination identifier may include an identification of an account at the remote system 818. The account may be associated with a subject that receives medicament therapy from the ambulatory medicament device 100. Further, the identification of the account may include account access information enabling the charging station 700 to access the account on behalf of the ambulatory medicament device 100 and/or the subject. For example, the identification of the account may include a username, password, or other account access information. The account access information may be stored at the memory 812. The processor 810 may access the account access information and transmit it to the charging station 700 enabling the charging station 700 to access the remote system 818. Although the account access information may be included as part of the destination identifier, in some cases, the account access information may be transmitted to the charging station 700 independent of the destination identifier. For example, in some cases, the destination may be known or previously registered with the charging station 700. In some such cases, the account access information may be provided to enable the charging station 700 to access the account of the subject at the remote system 818.

In some cases, the block 1014 may include encoding the ambulatory medicament device data. In such cases, the processor 810 may transmit the encoded ambulatory medicament device data to the charging station 700. Encoding the ambulatory medicament device data may include encapsulating the ambulatory medicament device data in one or more transmit packets addressed to the remote system 818. By encapsulating the transmit packets or data packets with the address of the remote system 818, the ambulatory medicament device data can be transmitted to the remote system 818 via the charging station 700 without the charging station 700 accessing the underlying data included in the encapsulated data packets thereby, improving privacy and security of the ambulatory medicament device data. Further, encoding the ambulatory medicament device data may include formatting the ambulatory medicament device data for transmission over the data connection. For example, the ambulatory medicament device data may be converted to a string of values understood by an application at the remote system 818 that reduces the size of the data. As another example, the processor 810 may chunk the ambulatory device data into particular sized data packets to facilitate transmission of the data. As previously described, in some cases, the charging station 700 may packetize or encode the ambulatory medicament device data. In such cases, the ambulatory medicament device 100 may provide access to the ambulatory medicament device data without encoding the data. In some embodiments, encoding the ambulatory medicament device data may include encrypting or otherwise securing the data using, for example, asymmetric encryption or a shared secret.

The process 1000 may be performed each time the ambulatory medicament device 100 is positioned with respect to the charging station 700 in a manner that permits charging of the battery 844 of the ambulatory medicament device 100. Alternatively, or in addition, the process 1000 may be performed at set times or after a particular amount of time has elapsed since prior performance of the process 1000. For example, assuming the battery 844 requires charging once a week, the process 1000 may be performed once a week when the ambulatory medicament device 100 is electrically connected to the charging station 700. Alternatively, although the ambulatory medicament device 100 may be charged once a week, the process 1000 may be performed once a month. In some cases, at least some of the process 1000 is performed each time a charge connection is detected, while some of the process 1000 is performed at particular times. As another example, the ambulatory medicament device 100 may be charged each time it is connected to the charging station 700, but data transfer may occur only when the charging station 700 has a connection to the network 828. In some cases, ambulatory medicament device data may be transmitted to the charging station 700 regardless of access to the network 828. This enables the charging station 700 to transmit the ambulatory medicament device data to the remote system 818 when a connection to the network 828 is established even when the ambulatory medicament device 100 is no longer connected to the charging station 700.

In some embodiments, the process 1000 may include preparing the ambulatory medicament device data for transmission before it is transmitted to the charging station 700. Preparing the ambulatory medicament device data for transmission may include the aforementioned encoding of the ambulatory medicament device data. Alternatively, or in addition, preparing the ambulatory medicament device data for transmission may include filtering certain subject identifying information, deduplicating data, chunking the data into particular sized data blocks, tagging the data with time stamps or subject identifying information, or any other process that may facilitate communication of the ambulatory medicament device data and/or processing of the ambulatory medicament device data by the charging station 700 or the remote system 818.

### Example Ambulatory Medicament Device Configuration Process

Typically, when an ambulatory medicament device 100 is manufactured, the control algorithm is not personalized to a particular subject. In some cases, the ambulatory medicament device 100 may be pre-configured by a user (e.g., a doctor, healthcare provider, the subject, or a guardian of the subject) to provide for better maintenance of the subject's disease by entering parameters that are specific to the subject or to a group of people that have the same disease as the subject. For example, a weight, an age, a gender, or clinical data obtained for the subject may be provided to the 100 upon first use or during an initialization procedure. Over time, embodiments of the ambulatory medicament device 100 can adapt or refine the control algorithm to improve maintenance of the subject's disease. Sometimes, the configuration process is repeated to account for physiological changes of the subject or to permit refinement by a healthcare provider of maintenances of the subject's disease. This may require the subject to make an appointment with the doctor, which is not always convenient. Further, if the ambulatory medicament device 100 is replaced, for example, due to availability of a new model or damage to the ambulatory medicament device 100, the process of initializing the ambulatory medicament device 100 or adapting the control algorithm to the subject may need to be repeated.

In certain embodiments, a configuration chip 814 can be configured with configuration data that can be used to facilitate configuration of the ambulatory medicament device 100. This configuration data may be specific to the subject. Moreover, in some cases, a doctor can select or modify the configuration data and store it on the configuration chip 814. This configuration chip 814 may then be provided to the subject, or another user, enabling the subject, or other user, to configure the ambulatory medicament device 100 at his or her convenience. Further, the configuration chip 814 can be used to configure replacement ambulatory medicament devices as needed. Moreover, the configuration chip 814 can be modified as changes in features of the ambulatory medicament device 100 available to the subject are modified (e.g., the addition of counter-regulatory agent control). In some cases, the configuration chip 814 may be programmed or modified with backup therapy protocol data that may be generated by the ambulatory medicament device 100 enabling a replacement ambulatory medicament device to begin with refinements to the control algorithm generated by the prior ambulatory medicament device 100. In some cases, the configuration chip 814 may be configured to trigger a rescues dose, a rapid or fast bolus, or any other dosing of a medicament. Further, the configuration chip 814 may be configured to enable or disable features. For example, the configuration chip 814 may be configured to enable or disable child-lock features that restrict the ability of a child or a subject with an intellectual disability from accessing or modifying features in an unsafe manner.

In some cases, the configuration chip 814 may be configured to initiate or agree to a software update. For example, if a user has agreed to install a new feature (e.g., via a purchased upgrade or otherwise), the user may receive the configuration chip 814. The user may then bring the configuration chip 814 within proximity of the ambulatory medicament device 100 to cause the software upgrade process or feature installation process to begin. The software upgrade may be received from the configuration chip 814, or the ambulatory medicament device 100 may communicate with a remote system 818 to obtain the software upgrade, or other software or features.

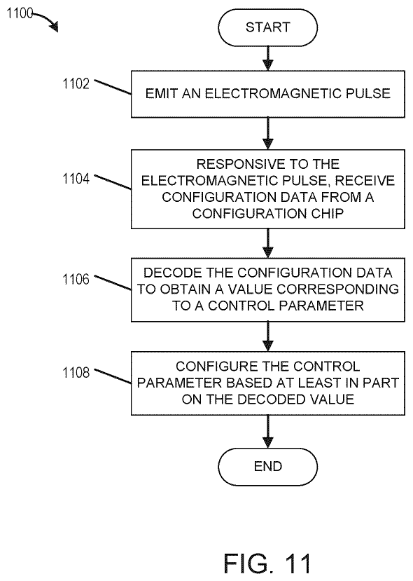

FIG. 11 illustrates a flowchart of an example ambulatory medicament device configuration process 1100 in accordance with certain embodiments. The process 1100 can be implemented by any system that can access configuration data from a configuration chip and configure an ambulatory medicament device based at least in part on the configuration data. The process 1100, in whole or in part, can be implemented by, for example, an ambulatory medicament device 100, a processor 810, a chip reader 808, a short-range transceiver 804, a data and charge port 806, or a glucose control system 510, among others. Although any number of systems, in whole or in part, can implement the process 1100, to simplify discussion, the process 1100 will be described with respect to particular systems.

The process may begin at block 1102 where, for example, the chip reader 808 emits an electromagnetic pulse. The electromagnetic pulse may be part of a discovery or scanning process for identifying a configuration chip 814 or other electronic device, such as the smartphone 816 within communication range of the ambulatory medicament device 100. In some cases, the electromagnetic pulse emission may be replaced with any other discovery or scanning process that may be used to identify a configuration chip 814 or other electronic device in communication range of the ambulatory medicament device 100. In some cases, a user interaction with a user interface of the ambulatory medicament device 100 causes the 808 to emit the electromagnetic pulse. For example, a user may turn on the chip reader 808 or initiate a discovery process by interacting with a user interface of the ambulatory medicament device 100. In some cases, the chip reader 808 may be part of the short-range transceiver 804 capable of performing a configuration chip 814 or other electronic device discovery process. For example, the chip reader 808 and/or the short-range transceiver 804 may be or may include a Bluetooth® chip that is capable of performing a Bluetooth discovery process.

In some cases, the chip reader 808 may emit the electromagnetic pulse in response to a proximity detection signal received in response to the configuration chip 814, or other electronic device, being within a threshold proximity of the chip reader 808. For example, a light sensor or infrared sensor may detect a change in a light or infrared signal. In response to the changing light or infrared signal, the chip reader 808 may be activated to emit the electromagnetic pulse or field. In some cases, the chip reader 808 may be or may include a proximity sensor.

At block 1104, responsive to the electromagnetic pulse, the chip reader 808 receives configuration data from a configuration chip 814. The configuration data may be or may include a configuration code that encodes one or more values corresponding to one or more control parameters. The control parameters may be used by a control algorithm that controls medicament therapy provided to the subject. The control algorithm may generate a dose control signal to cause the ambulatory medicament device 100 to administer a quantity of medicament from a medicament reservoir into a subject based at least in part on glucose level data or other clinical data measured or determined for the subject, and/or based on user input received at the ambulatory medicament device 100. The configuration code may be of a specific length. For example, the configuration code may have a length of 16 numeric or alphanumeric characters. In some cases, the configuration code may be longer or shorter than 16 characters. As another example, the configuration code may have a length of 2 kilobytes or 4 kilobytes. In some cases, the configuration code may be longer or shorter than 2 or 4 kilobytes. In some cases, the length of the configuration code may be limited by the type of chip used for the configuration chip 814. For example, a passive or semi-passive configuration chip 814 may be more limited in data storage capacity than an active configuration chip. Generally, it is desirable to use a passive configuration chip 814 to ensure that configuration chip 814 can be used at any time without concern for available power. However, in some cases, an active configuration chip 814 may be used. Using an active configuration chip 814 may enable the configuration chip 814 to store more data and/or to have increased processing capabilities. In some cases, the active configuration chip 814 may be rechargeable and may be capable of being charged by the charging station 700. Embodiments of generating and using the configuration code are described in more detail in International Application No. PCT/US21/72742, which is incorporated by reference above, and in U.S. Provisional Application No. 63/261,290, the disclosure of which is hereby incorporated by reference in its entirety herein for all purposes.

In some implementations, the configuration data may include at least one of a plurality of encoded dosing parameters. The plurality of encoded dosing parameters may correspond to a set of dosing parameters used by the control algorithm. The dosing parameters may include a correction dosing parameter used by the control algorithm to determine a correction bolus of medicament to administer to the subject. Another dosing parameter may include a food intake dosing parameter used by the control algorithm to determine a food intake bolus size of medicament to administer to the subject. Alternatively, or in addition, the food intake dosing parameter may be used by the control algorithm to determine the distribution of medicament to administer to the subject over time in response to a meal announcement. For example, the food intake dosing parameter may indicate or may be used to determine that 60% of a food intake bolus be administered immediately and 40% be administered within thirty minutes of the food intake meal announcement. As another example, the food intake dosing parameter may indicate or may be used to determine that 100% of a food intake bolus be administered immediately when the food intake announcement is for a small meal or a snack, but that the food intake bolus be distributed over time when the food intake announcement is for a normal or larger meal. The dosing parameters may further include a basal dosing parameter used by the control algorithm to determine a basal rate of medicament to administer to the subject.

As indicated above, the configuration data may include a backup therapy protocol. This backup therapy protocol may be generated by the ambulatory medicament device 100, another ambulatory medicament device previously used by the subject, by a healthcare provider, or by any other source of a backup therapy protocol. Embodiments of generating a backup therapy protocol are described in U.S. Pat. No. 10,960,137 and U.S. Publication No. 2021/0213200, the disclosures of which are hereby incorporated by reference in their entirety herein for all purposes. In some cases, the configuration data may be associated with or specific to the subject. In other cases, the configuration data may be associated with a demographic that includes the subject. For example, the configuration data may be associated with subjects of the same age, gender, weight, puberty status, or other physiological parameters that may affect the status or maintenance of their disease.

At block 1106, the processor 810 decodes the configuration data to obtain a value corresponding to a control parameter. Decoding the configuration data may include decrypting the configuration data. In some cases, decrypting the configuration data may involve using a decryption key generated or determined at the time the configuration chip 814 was programmed. This decryption key may be specific to the subject and may be stored at the memory 812 or at the remote system 818. Advantageously, the use of encryption may prevent the configuration chip 814 from being used to program an ambulatory medicament device not associated with the subject. In some cases, the encryption may be a hash that is based at least in part on a shared hash function or shared value. Further, the processor 810 may verify a checksum of the configuration data prior to or as part of decoding the configuration data. Verifying the checksum may including performing a hash function on at least a portion of the configuration data. The checksum may be used to ensure that the configuration data has not been modified in transit. Further, the checksum may be used to ensure that the configuration data is useable with the ambulatory medicament device 100 or with a particular model or type of ambulatory medicament device.

In some cases, the value may be an initial value for the control parameter. The control parameter may be adapted over a time period based at least in part on medicament therapy administered over the time period. The time period may be a set or defined time period, or it may be an ongoing time period. The control parameter may be adapted using any of the embodiments disclosed in the Controller Disclosures and in the additional disclosures previously incorporated by reference herein. Thus, although the control parameter may be configured based on or set to the value initially, the value of the control parameter may change over time. In some cases, the value may replace an initial value or prior value of the control parameter.

In some embodiments, the processor 810 may verify that a reference code of the ambulatory medicament device 100 matches a reference code of the configuration data prior to configuring the control parameter of the control algorithm. The reference code may be any code that can be used to confirm that the value of the control parameter is associated with or intended for use with the ambulatory medicament device 100 of a particular type or a particular subject or group of subjects. For example, an ambulatory medicament device 100 configured for use with type 1 diabetics may have a first reference code while an ambulatory medicament device 100 associated for use with type 2 diabetics may have a second reference code. If the value of the control parameter received from the configuration chip 814 is intended for ambulatory medicament devices being used by type 1 diabetics, the configuration chip 814 may include the reference code for type 1 diabetics. Conversely, if the value of the control parameter received from the configuration chip 814 is intended for ambulatory medicament devices being used by type 2 diabetics, the configuration chip 814 may include the reference code for type 2 diabetics. Thus, the processor 810 of the ambulatory medicament device 100 may compare its reference code to that of the configuration chip 814 to determine whether the control parameter can or should be configured with the value stored on the configuration chip 814. As another example, the configuration chip 814 may include a reference value or code associated with the subject. If the processor 810 determines that the reference value matches a corresponding reference value or code of the ambulatory medicament device 100 it may be determined that the configuration chip 814 has been configured for the subject. Otherwise, the ambulatory medicament device 100 may determine that the configuration chip 814 is not approved or authorized for use by the subject (e.g., the configuration chip was inadvertently switched with another subject's configuration chip 814). In some cases, the reference code may be used to ensure that the ambulatory medicament device 100 is operating a particular software version or control algorithm before modifying the control parameter.

The reference code may include any type of value that may be used to verify the configuration chip 814 or configuration data stored on the configuration chip 814. For example, the reference code may include at least a portion of a serial number (e.g., a portion associated with a model of the ambulatory medicament device 100), a software version number, or a model number. This reference code may then be compared to a corresponding code stored in the memory 812 of the ambulatory medicament device 100. If the reference code matches the corresponding code of the ambulatory medicament device 100, then it may be determined that the ambulatory medicament device 100 can be modified based on the value obtained from the configuration chip 814.

At block 1108, the processor 810 configures the control parameter based at least in part on the decoded value. As stated above, the control parameter may be used by a control algorithm that controls medicament therapy provided to the subject. The control parameter may be any type of control parameter that can modify the provided medicament therapy. In some cases, the control parameter relates to the delivery of insulin, a counter-regulatory agent (e.g., Glucagon), or any other type of medicament that may be used to treat diabetes or other diseases managed at least in part by an ambulatory medicament device 100.

Configuring the control parameter may include modifying an existing value for the control parameter based on the decoded value. Modifying the existing value may include increasing or decreasing the existing value, selecting an alternative value, or any other process for modifying the control parameter value. In some cases, configuring the control parameter may include setting the control parameter to the decoded value.

In some cases, configuring the control parameter may trigger an immediate action. For example, configuring the control parameter may trigger administering of a rescue dose of the counter-regulatory agent, or a fast or rapid dose of a medicament. In some cases, the triggering of the immediate action may cause additional modifications to a control parameter to account for the effects of the immediate action. In other cases, the control algorithm may implicitly account for the effects of the immediate action by responding to changes in the subject's glucose level and/or medicament on board values. In some cases, configuring the control parameter may modify operation of, or enable modification of, the control algorithm of the ambulatory medicament device 100, which may or may not result in a change or an immediate change of medicament therapy. For example, in some cases, the control parameter change may result in a modification of current or future medicament delivery. In other cases, the control parameter change may result in a modification of a presentation of a user interface but may or may not result in a modification of current or future medicament delivery. The modification of the presentation of the user interface may be cosmetic, or it may be substantive enabling, for example, new features or new feature control. For example, the presentation of the user interface may enable the subject to modify medicament therapy settings not previously available on the ambulatory medicament device 100 or accessible by the user or subject.

Changes to the control parameter based on the decoded value may be permanent or may last at least until a subsequent change reverts or further modifies a value of the control parameter. Alternatively, the change to the control parameter may be valid for a set time period or until a trigger occurs. For example, the change to the control parameter may be configured to last for a day, half a day, a week, until an infusion set site is modified, until a counter-regulatory agent injection occurs, or any other trigger that may result in the control parameter reverting to a prior value or to a new value.

In some embodiments, the configuration chip 814 may be used to unlock features of the ambulatory medicament device 100. For example, the configuration chip 814 may enable one or more features using embodiments of safe access controls described in U.S. Provisional Application No. 63/169,112, and in International Application No. PCT/US21/72742, the disclosures of which are previously incorporated by reference above. As another example, the configuration chip 814 may be used to unlock the ambulatory medicament device 100 using one or more of the embodiments described in U.S. Pat. No. 11,135,365, which is hereby incorporated by reference in its entirety for all purposes herein. For instance, the configuration chip 814 may be configured with a security code that, when accessed by the ambulatory medicament device 100 via the chip reader 808, may unlock access to the ambulatory medicament device 100 enabling a user to modify one or more control parameters of the ambulatory medicament device 100 via a user interface of the ambulatory medicament device 100 or a user interface of an electronic device in communication with the ambulatory medicament device 100. Similarly, the configuration chip 814 may include a configuration code that enables access to one or more features of the ambulatory medicament device 100, which may or may not have been previously accessible by the subject, or a user caring for the subject.

### Additional Embodiments

Near-field communication (NFC) can be used for data transmission and communications. This disclosure identifies some use cases for NFC on an ambulatory pump or a ambulatory medicament device 100.

Both a smart charger (e.g., the charging station 700) and a pump (e.g., an ambulatory medicament device 100) can include an active data communications interface (e.g., NFC, such as STMicro short-range, high-bandwidth communication solution in addition to NFC) on both the pump and the charger. Alternatively, the charger and the pump can use Bluetooth or a wired connection instead of or in addition to NFC. When the pump is placed on a charger, the pump may transmit data to the charger which may have LTE/WiFi or other connection to a cloud server or other remote server and can transmit data to the cloud server. When a visitor or guest uses the charging station, the charging station can reject unknown pumps, or access can be shared to multiple known (e.g., registered with pump serial number) or guest users. Although a visitor pump may be rejected for data communications, charging may still occur.

The pump when placed on the charger can transfers clinical data (therapy deliveries, glucose data, etc.) and engineering data (alarm data, user interactions with pump, etc.) to a remote server via a communications bridge (e.g., a pass-through interface) integrated with the charging base. The charging base can have a Wi-fi transceiver, a wired (e.g., Ethernet) connection, or an LTE transceiver for connecting to the Internet. The charging base can be configured using, e.g., a smartphone app, or directly from the ambulatory medicament device 100 (e.g., by selecting SSID, entering password, adding customer account information, etc.). The charging base can receive software updates and transfer the updates to the pump.

Advantageously, the smart charger may be simpler and cheaper to implement than integrating an LTE transceiver (or other wide-area network transceiver) with the pump. Further, integrating the LTE transceiver with the charging station 700 can reduce power and cost compared to integrating the transceiver with the ambulatory medicament device 100.

Customer account information can be registered with smart charger to allow authentication of the smart charger.

The ambulatory medicament device 100 can communicate with a charge pad (e.g., the charging station 700) via NFC or Bluetooth Low Energy (BLE) and the charge pad can send data to cloud using either LTE or Wi-Fi when the ambulatory medicament device 100 is placed on the charge pad In some cases, the ambulatory medicament device 100 can communicate with a power brick plugged into a wall adapter over BLE and the adapter can communicate with the cloud using LTE or Wi-Fi. The user can have multiple wall adapters in the house.

When using a Wi-Fi interface, the SSID(s) and Password(s) can be programmed into the user interface of the ambulatory medicament device 100. The communication device may for SSIDs, communicate to the ambulatory medicament device 100 and allow the user to select, or the user may enter the SSID (as may be required for a hidden SSID). Additionally, the password may be entered into the ambulatory medicament device 100. The SSID(s) and associated password(s) may be stored in the ambulatory medicament device 100 and/or the communication device or the charging station 700.

Further, a programming fob can be used to configure the ambulatory medicament device 100. The programming fob may be implemented as a passive chip (read-only or read/write) programmed by a manufacturer of the ambulatory medicament device 100 or other authorized user. The passive chip can be provided to a user and can include set-up parameters (e.g., body weight, operating mode (e.g., type 1 diabetes or type 2 diabetes), pump name, basal rates, correction factor, carbohydrate ratio, alarm preferences, setpoint targets, prescription, etc.) that can be programmed by the pump manufacturer, fob manufacturer, healthcare provider, the pump itself, or any other user that can program the fob based on a prescription and/or user inputs, and that can provide the fob to the subject or patient. When a new pump is received, a user can bring the chip or programming fob into proximity of the pump which can read the fob and initialize the pump based on set-up parameters stored on the fob. Additionally, if a pump is factory reset or a replacement pump is received, the chip or programming fob can be used to initialize or re-initialize the pump. In some cases, the programming fob may be read-only. In such cases, once configured, the programming fob cannot be modified. In other cases, the programming fob may be a read/write chip. In some such embodiments, pump state data can be written to the chip when in range of the ambulatory medicament device 100. Personal health data stored on the chip can be encrypted. The use of a read/write chip can be useful for patch pumps that may be replaced frequently. Using the programming fob, each new patch pump can be programmed with subject specific control parameters or an updated control algorithm based on therapy provided to the subject. In some cases, the chip can be integrated with the pump, and transfers of health data or control parameter values can be accomplished by holding two pumps in proximity to each other.

NFC chips may also be used as a passkey, such as to open locked doors. Further, the programming fob can be used in conjunction with a child lock to enable parental controls. The fob can also authenticate or authorize the ambulatory medicament device 100 to update software or enable advanced features. In some cases, the programming fob (or configuration chip 814 as referred to herein) can be attachable to a key ring. In some cases, the configuration chip 814 may be replaced by a cell phone NFC circuit as may be included in smartphone 816, for example. Further, the configuration chip 814 or smartphone 816 may be used as a security mechanism to enable features on the ambulatory medicament device 100.

In some cases, a fob or configuration chip 814 may be provided to a parent or caregiver to authorize a rescue bolus. For example, if a hypoglycemic event occurs, the configuration chip 814 may be brought into range of the ambulatory medicament device 100 to trigger a rescue dose of glucagon. The parent fob can enable safe access level features (e.g., glucagon rescue dose, other commands, or command sequences) that are otherwise unavailable. For example, the ability to adjust a setpoint target range may be unavailable for the ambulatory medicament device 100. However, if it is determined that an authorized user is attempting to adjust the setpoint range, a setpoint range adjustment feature may be made available. This determination may be made, at least in part, by the detection and/or accessing of the parent fob or configuration chip 814. The fob can include a button or other UI element to ensure that commands do not trigger inadvertently.

The passive chip or configuration chip 814 can be programmed by a mobile device (e.g., the smartphone 816) or an ambulatory medicament device 100. In some cases, a removable passive chip can be part of the charging station 700. When the pump is placed on the charger and/or at regular intervals (e.g., once per week) the pump can re-program the configuration chip 814 with or based on the state data of the ambulatory medicament device 100. Further, the configuration chip 814 may be configured with or to include a back-up therapy protocol. When a new pump is placed on the charger, the pump can be initialized by the configuration chip 814.

### Example Embodiments

Some non-limiting example enumerated embodiments are recited as clauses in this section in the form of methods, systems, and non-transitory computer-readable media, without limitation. Except where contradictory, each of the dependent clauses below may depend on any one of the prior clauses in a multiply dependent manner. Further, except where contradictory, any one of the dependent clauses below may depend from a different independent clause listed below.

Clause 1. A charging station configured to receive ambulatory medicament device data from an ambulatory medicament device, the charging station comprising: a charging assembly configured to charge a battery of the ambulatory medicament device; a memory configured to at least store specific computer-executable instructions; a first transceiver configured to at least receive the ambulatory medicament device data from the ambulatory medicament device; a second transceiver configured to at least transmit the ambulatory medicament device data received from the ambulatory medicament device; and a hardware processor in communication with the memory and configured to execute the specific computer-executable instructions to at least: detect a charge connection to the ambulatory medicament device; initiate charging of the ambulatory medicament device using the charging assembly; determine a destination identifier for the ambulatory medicament device data; receive, via the first transceiver, the ambulatory medicament device data from the ambulatory medicament device; and transmit, using the second transceiver, the ambulatory medicament device data to a remote system corresponding to the destination identifier.

Clause 2. The charging station of clause 1, wherein the first transceiver and the second transceiver support different communication protocols.

Clause 3. The charging station of clause 1, wherein the first transceiver comprises a near-field transceiver or a short-range transceiver.

Clause 4. The charging station of clause 3, wherein the first transceiver comprises a near-field communication transceiver, a Wi-Fi HaLow transceiver, a Zigbee transceiver, a Z-wave transceiver, a Bluetooth transceiver, a Bluetooth Low Energy transceiver, or a custom short-range transceiver.

Clause 5. The charging station of clause 1, wherein the second transceiver comprises a wide-area network transceiver or a cellular network transceiver.

Clause 6. The charging station of clause 1, wherein the charging assembly comprises a wireless charging assembly.

Clause 7. The charging station of clause 6, wherein detecting the charge connection comprises detecting a wireless charge connection with the wireless charging assembly.

Clause 8. The charging station of clause 1, wherein the charging assembly comprises an inductive charging circuit configured to inductively charge the ambulatory medicament device.

Clause 9. The charging station of clause 1, wherein the ambulatory medicament device is registered with the charging station.

Clause 10. The charging station of clause 1, wherein the hardware processor is further configured to: select data to send to or receive from the ambulatory medicament device using one or more of a plurality of data selection criteria, wherein the plurality of data selection criteria comprises: a determination that an application update comprising an update to an application executing on the ambulatory medicament device is available; a determination that an updated control parameter comprising a control parameter value different from one stored on the ambulatory medicament device is available; a determination that access to an application feature of the application is permitted; a determination that an alarm has occurred at the ambulatory medicament device; a determination that device data of the ambulatory medicament device indicates occurrence of a malfunction at the ambulatory medicament device; a determination that a time period has elapsed; a determination that a time is a scheduled data transfer time; a determination that a request for the ambulatory medicament device data has been received; a determination that clinical data does not satisfy expected clinical data; a determination that user interface data does not satisfy an expected user interaction profile; and a determination that a backup therapy protocol has been generated or modified.

Clause 11. The charging station of clause 1, wherein the ambulatory medicament device data comprises one or more of clinical data corresponding to medicament therapy provided by the ambulatory medicament device, device data corresponding to operation of the ambulatory medicament device, or user interface data corresponding to user interaction with the ambulatory medicament device.

Clause 12. The charging station of clause 1, wherein the hardware processor is further configured to execute the specific computer-executable instructions to at least obtain a first unique identifier associated with the ambulatory medicament device, wherein the destination identifier is determined based at least in part on the first unique identifier associated with the ambulatory medicament device.

Clause 13. The charging station of clause 12, wherein the first unique identifier comprises a serial number of the ambulatory medicament device.

Clause 14. The charging station of clause 12, wherein the first unique identifier comprises a subject identifier associated with a subject that receives medicament therapy from the ambulatory medicament device.

Clause 15. The charging station of clause 1, wherein the hardware processor is further configured to: detect a charge connection to a second ambulatory medicament device; obtain a second unique identifier associated with the second ambulatory medicament device; determine based at least in part on the second unique identifier that the second ambulatory medicament device is not registered with the charging station; and initiate charging of the second ambulatory medicament device without accessing second ambulatory medicament device data from the second ambulatory medicament device.

Clause 16. The charging station of clause 1, wherein the charging assembly comprises a universal serial bus plug or a universal serial bus port, and wherein the first transceiver comprises a universal serial bus transceiver in communication with the universal serial bus plug or the universal serial bus port.

Clause 17. The charging station of clause 1, wherein the destination identifier comprises an identification of an account at the remote system, and wherein the account is associated with a subject that receives medicament therapy from the ambulatory medicament device.

Clause 18. The charging station of clause 1, wherein the hardware processor is further configured to encode the ambulatory medicament device data to obtain encoded data.

Clause 19. The charging station of clause 18, wherein transmitting the ambulatory medicament device data to the remote system comprises transmitting the encoded data.

Clause 20. The charging station of clause 18, wherein encoding the ambulatory medicament device data comprises encapsulating the ambulatory medicament device data in one or more transmit packets addressed to the remote system.

Clause 21. The charging station of clause 18, wherein encoding the ambulatory medicament device data comprises formatting the ambulatory medicament device data for transmission over a wide area network or a cellular network.

Clause 22. The charging station of clause 1, wherein the hardware processor is further configured to transmit the ambulatory medicament device data to the remote system by at least: using account access information to access an account associated with a subject that receives medicament therapy from the ambulatory medicament device, wherein the account access information is associated with the subject; and associating the ambulatory medicament device data with the account at the remote system.

Clause 23. The charging station of clause 22, wherein the memory is further configured to at least store the account access information.

Clause 24. The charging station of clause 22, wherein the account access information is obtained during a registration process that registers the ambulatory medicament device with the charging station.

Clause 25. The charging station of clause 1, wherein the memory is further configured to store the ambulatory medicament device data at least until a connection to the remote system is available.

Clause 26. The charging station of clause 1, wherein the memory is further configured to store data to transmit to the ambulatory medicament device at least until a data connection to the ambulatory medicament device is established.

Clause 27. The charging station of clause 26, wherein the data comprises: an application update comprising an update to an application executing on the ambulatory medicament device; an updated control parameter comprising a control parameter value different from one stored on the ambulatory medicament device; an access permissions update comprising an update to access permissions of a user of the ambulatory medicament device; or medicament device data from a second ambulatory medicament device.

Clause 28. The charging station of clause 1, wherein the memory is further configured to buffer the ambulatory medicament device data when receiving the ambulatory medicament device data from the ambulatory medicament device data, when transmitting the ambulatory medicament device data to the remote system, or both when receiving the ambulatory medicament device data from the ambulatory medicament device data and when transmitting the ambulatory medicament device data to the remote system.

Clause 29. A computer-implemented method of receiving ambulatory medicament device data from an ambulatory medicament device by a charging station comprising a charging assembly configured to charge a battery of the ambulatory medicament device; a first transceiver configured to at least receive the ambulatory medicament device data from the ambulatory medicament device; and a second transceiver configured to at least transmit the ambulatory medicament device data received from the ambulatory medicament device, the computer-implemented method comprising: by a hardware processor of the charging station, detecting a charge connection to the ambulatory medicament device; initiating charging of the ambulatory medicament device using the charging assembly; determining a destination identifier for the ambulatory medicament device data; receiving, via the first transceiver, the ambulatory medicament device data from the ambulatory medicament device; and transmitting, using the second transceiver, the ambulatory medicament device data to a remote system corresponding to the destination identifier.

Clause 30. The computer-implemented method of clause 29, wherein the first transceiver and the second transceiver support different communication protocols.

Clause 31. The computer-implemented method of clause 29, wherein the first transceiver comprises a near-field transceiver or a short-range transceiver.

Clause 32. The computer-implemented method of clause 31, wherein the first transceiver comprises a near-field communication transceiver, a Wi-Fi HaLow transceiver, a Zigbee transceiver, a Z-wave transceiver, or a custom short-range transceiver.

Clause 33. The computer-implemented method of clause 29, wherein the second transceiver comprises a wide-area network transceiver or a cellular network transceiver.

Clause 34. The computer-implemented method of clause 29, wherein the charging assembly comprises a wireless charging assembly.

Clause 35. The computer-implemented method of clause 34, wherein detecting the charge connection comprises detecting a wireless charge connection with the wireless charging assembly.

Clause 36. The computer-implemented method of clause 29, wherein the charging assembly comprises an inductive charging circuit configured to inductively charge the ambulatory medicament device.

Clause 37. The computer-implemented method of clause 29, wherein the ambulatory medicament device is registered with the charging station.

Clause 38. The computer-implemented method of clause 29, further comprising selecting data to send to or receive from the ambulatory medicament device using one or more of a plurality of data selection criteria, wherein the plurality of data selection criteria comprises: a determination that an application update comprising an update to an application executing on the ambulatory medicament device is available; a determination that an updated control parameter comprising a control parameter value different from one stored on the ambulatory medicament device is available; a determination that access to an application feature of the application is permitted; a determination that an alarm has occurred at the ambulatory medicament device; a determination that device data of the ambulatory medicament device indicates occurrence of a malfunction at the ambulatory medicament device; a determination that a time period has elapsed; a determination that a time is a scheduled data transfer time; a determination that a request for the ambulatory medicament device data has been received; a determination that clinical data does not satisfy expected clinical data; a determination that user interface data does not satisfy an expected user interaction profile; and a determination that a backup therapy protocol has been generated or modified.

Clause 39. The computer-implemented method of clause 29, wherein the ambulatory medicament device data comprises one or more of clinical data corresponding to medicament therapy provided by the ambulatory medicament device, device data corresponding to operation of the ambulatory medicament device, or user interface data corresponding to user interaction with the ambulatory medicament device.

Clause 40. The computer-implemented method of clause 29, further comprising obtaining a first unique identifier associated with the ambulatory medicament device, wherein the destination identifier is determined based at least in part on the first unique identifier associated with the ambulatory medicament device.

Clause 41. The computer-implemented method of clause 40, wherein the first unique identifier comprises a serial number of the ambulatory medicament device.

Clause 42. The computer-implemented method of clause 40, wherein the first unique identifier comprises a subject identifier associated with a subject that receives medicament therapy from the ambulatory medicament device.

Clause 43. The computer-implemented method of clause 29, further comprising: detecting a charge connection to a second ambulatory medicament device; obtaining a second unique identifier associated with the second ambulatory medicament device; determining based at least in part on the second unique identifier that the second ambulatory medicament device is not registered with the charging station; and initiating charging of the second ambulatory medicament device without accessing second ambulatory medicament device data from the second ambulatory medicament device.

Clause 44. The computer-implemented method of clause 29, wherein the charging assembly comprises a universal serial bus plug or a universal serial bus port, and wherein the first transceiver comprises a universal serial bus transceiver in communication with the universal serial bus plug or the universal serial bus port.

Clause 45. The computer-implemented method of clause 29, wherein the destination identifier comprises an identification of an account at the remote system, and wherein the account is associated with a subject that receives medicament therapy from the ambulatory medicament device.

Clause 46. The computer-implemented method of clause 29, further comprising encoding the ambulatory medicament device data to obtain encoded data.

Clause 47. The computer-implemented method of clause 46, wherein transmitting the ambulatory medicament device data to the remote system comprises transmitting the encoded data.

Clause 48. The computer-implemented method of clause 46, wherein encoding the ambulatory medicament device data comprises encapsulating the ambulatory medicament device data in one or more transmit packets addressed to the remote system.

Clause 49. The computer-implemented method of clause 46, wherein encoding the ambulatory medicament device data comprises formatting the ambulatory medicament device data for transmission over a wide area network or a cellular network.

Clause 50. The computer-implemented method of clause 29, wherein said transmitting the ambulatory medicament device data to the remote system comprises: using account access information to access an account associated with a subject that receives medicament therapy from the ambulatory medicament device, wherein the account access information is associated with the subject; and associating the ambulatory medicament device data with the account at the remote system.

Clause 51. The computer-implemented method of clause 50, further comprising accessing the account access information from a memory of the charging station configured to store the account access information.

Clause 52. The computer-implemented method of clause 50, further comprising obtaining the account access information during a registration process that registers the ambulatory medicament device with the charging station.

Clause 53. The computer-implemented method of clause 29, further comprising storing the ambulatory medicament device data in a memory of the charging station at least until a connection to the remote system is available.

Clause 54. The computer-implemented method of clause 29, further comprising receiving data to transmit to the ambulatory medicament device from the remote system.

Clause 55. The computer-implemented method of clause 54, wherein the data comprises: an application update comprising an update to an application executing on the ambulatory medicament device; an updated control parameter comprising a control parameter value different from one stored on the ambulatory medicament device; an access permissions update comprising an update to access permissions of a user of the ambulatory medicament device; or medicament device data from a second ambulatory medicament device.

Clause 56. An ambulatory medicament device configured to transmit ambulatory medicament device data to a charging station, the ambulatory medicament device comprising: a battery configured to power the ambulatory medicament device; a charging circuit configured to receive power from the charging station and to charge the battery; a memory configured to at least store the ambulatory medicament device data and specific computer-executable instructions; a transceiver configured to at least transmit the ambulatory medicament device data to the charging station; and a hardware processor in communication with the memory and configured to execute the specific computer-executable instructions to at least: detect a charge connection to the charging station; initiate, via the charging circuit, charging of the battery over the charge connection; determine whether the charging station supports a data connection to the ambulatory medicament device; and in response to determining that the charging station supports the data connection: establish the data connection to the charging station; automatically select, without user interaction with a user interface, ambulatory medicament device data using one or more of a plurality of data selection criteria; prepare the ambulatory medicament device data for transmission to the charging station via the data connection; and automatically transmit, without user interaction with the user interface, the ambulatory medicament device data to the charging station enabling communication of the ambulatory medicament device data by the charging station to a remote system separate from the charging station without the ambulatory medicament device establishing communication with the remote system.

Clause 57. The ambulatory medicament device of clause 56, wherein the transceiver comprises a near-field transceiver or a short-range transceiver.

Clause 58. The ambulatory medicament device of clause 57, wherein the transceiver comprises a near-field communication transceiver, a Wi-Fi HaLow transceiver, a Zigbee transceiver, a Z-wave transceiver, or a custom short-range transceiver.

Clause 59. The ambulatory medicament device of clause 56, wherein the charging circuit comprises a wireless charging circuit configured to interface with a wireless charging assembly of the charging station.

Clause 60. The ambulatory medicament device of clause 56, wherein the charging circuit comprises an inductive charging circuit configured to inductively receive power from the charging station.

Clause 61. The ambulatory medicament device of clause 56, wherein the hardware processor is further configured to register the ambulatory medicament device with the charging station.

Clause 62. The ambulatory medicament device of clause 61, wherein registering the ambulatory medicament device with the charging station comprises providing to the charging station a unique identifier associated with the ambulatory medicament device or a subject that receives therapy from the ambulatory medicament device.

Clause 63. The ambulatory medicament device of clause 61, wherein registering the ambulatory medicament device with the charging station comprises providing, to the charging station, access information for accessing the remote system on behalf of a subject that receives therapy from the ambulatory medicament device.

Clause 64. The ambulatory medicament device of clause 56, wherein the plurality of data selection criteria comprises: a determination that an alarm has occurred at the ambulatory medicament device; a determination that device data of the ambulatory medicament device indicates occurrence of a malfunction at the ambulatory medicament device; a determination that a time period has elapsed; a determination of a time of a previous transmission of previous ambulatory medicament device data; a determination that a request for the ambulatory medicament device data has been received; a determination that clinical data does not satisfy expected clinical data; a determination that user interface data does not satisfy an expected user interaction profile; and a determination that a backup therapy protocol has been generated or modified.

Clause 65. The ambulatory medicament device of clause 56, wherein the ambulatory medicament device data comprises one or more of clinical data corresponding to medicament therapy provided by the ambulatory medicament device, device data corresponding to operation of the ambulatory medicament device, or user interface data corresponding to user interaction with the ambulatory medicament device.

Clause 66. The ambulatory medicament device of clause 56, wherein the hardware processor is further configured to: detect a charge connection to a second charging station; determine that a data connection to the second charging station is not authorized; and initiate, via the charging circuit, charging of the battery over the charge connection to the second charging station without permitting transmission of the ambulatory medicament device data to the second charging station.

Clause 67. The ambulatory medicament device of clause 56, further comprising a universal serial bus plug or a universal serial bus port, wherein the transceiver comprises a universal serial bus transceiver in communication with the universal serial bus plug or the universal serial bus port, and wherein the charge connection is established via the universal serial bus plug or the universal serial bus port.

Clause 68. The ambulatory medicament device of clause 56, wherein transmitting the ambulatory medicament device data to the charging station further comprises transmitting a destination identifier corresponding to the remote system to the charging station.

Clause 69. The ambulatory medicament device of clause 68, wherein the destination identifier comprises an identification of an account at the remote system, and wherein the account is associated with a subject that receives medicament therapy from the ambulatory medicament device.

Clause 70. The ambulatory medicament device of clause 56, wherein the hardware processor is further configured to encode the ambulatory medicament device data to obtain encoded data.

Clause 71. The ambulatory medicament device of clause 70, wherein transmitting the ambulatory medicament device data to the charging station comprises transmitting the encoded data.

Clause 72. The ambulatory medicament device of clause 70, wherein encoding the ambulatory medicament device data comprises encapsulating the ambulatory medicament device data in one or more transmit packets addressed to the remote system.

Clause 73. The ambulatory medicament device of clause 70, wherein encoding the ambulatory medicament device data comprises formatting the ambulatory medicament device data for transmission over the data connection.

Clause 74. The ambulatory medicament device of clause 56, wherein the hardware processor is further configured to: access account access information associated with an account of a subject at the remote system, wherein the subject receives medicament therapy from the ambulatory medicament device; and transmit the account access information to the charging station enabling the charging station to access the account of the subject at the remote system.

Clause 75. The ambulatory medicament device of clause 74, wherein the memory is further configured to at least store the account access information.

Clause 76. The ambulatory medicament device of clause 56, wherein the hardware processor is further configured to determine that the data connection to the charging station is authorized.

Clause 77. The ambulatory medicament device of clause 76, wherein the data connection to the charging station is established in response to determining that the charging station supports the data connection and that the data connection to the charging station is authorized.

Clause 78. The ambulatory medicament device of clause 76, wherein the hardware processor determines that the data connection to the charging station is authorized by at least: accessing a unique identifier of the charging station; and determining that the unique identifier of the charging station is registered with the ambulatory medicament device.

Clause 79. The ambulatory medicament device of clause 56, wherein the data connection comprises a secure connection using asymmetric encryption.

Clause 80. The ambulatory medicament device of clause 56, wherein at least one of the plurality of data selection criteria is based at least in part on a prior transmission time corresponding to a prior transmission of prior ambulatory medicament device data.

Clause 81. The ambulatory medicament device of clause 80, wherein the prior transmission of prior ambulatory medicament device data is to the remote system.

Clause 82. The ambulatory medicament device of clause 80, wherein the prior transmission of prior ambulatory medicament device data is to the charging station or a second charging station.

Clause 83. The ambulatory medicament device of clause 80, wherein the prior transmission time is stored in the memory of the ambulatory medicament device.

Clause 84. The ambulatory medicament device of clause 80, wherein the hardware processor is further configured to access the prior transmission time from the charging station.

Clause 85. The ambulatory medicament device of clause 80, wherein the hardware processor is further configured to transmit a query request to the charging station to query the remote system for the prior transmission time.

Clause 86. The ambulatory medicament device of clause 56, wherein at least one of the plurality of data selection criteria is based at least in part on a determination of prior ambulatory medicament device data transmitted to the charging station or to the remote system.

Clause 87. The ambulatory medicament device of clause 86, wherein the determination of the prior ambulatory medicament device data transmitted to the charging station or to the remote system is determined by accessing a communication record stored in the memory of the ambulatory medicament device.

Clause 88. The ambulatory medicament device of clause 86, wherein the determination of the prior ambulatory medicament device data transmitted to the charging station or to the remote system is determined by transmitting a query to the charging station to receive an indicator corresponding to at least some of the prior ambulatory medicament device data.

Clause 89. The ambulatory medicament device of clause 88, wherein the indicator comprises a confirmation of successful transmission of the prior ambulatory medicament device data to the remote system.

Clause 90. A computer-implemented method of transmitting ambulatory medicament device data to a charging station by an ambulatory medicament device comprising a charging circuit configured to receive power from the charging station and to charge a battery of the ambulatory medicament device, and a transceiver configured to at least transmit the ambulatory medicament device data to the charging station, the computer-implemented method comprising: by a hardware processor of the ambulatory medicament device, detecting a charge connection to the charging station; initiating, via the charging circuit, charging of the battery over the charge connection; determining whether the charging station supports a data connection to the ambulatory medicament device; and in response to determining that the charging station supports the data connection: establishing the data connection to the charging station; automatically selecting, without user interaction with a user interface, ambulatory medicament device data using one or more of a plurality of data selection criteria; preparing the ambulatory medicament device data for transmission to the charging station via the data connection; and automatically transmitting, without user interaction with the user interface, the ambulatory medicament device data to the charging station enabling communication of the ambulatory medicament device data by the charging station to a remote system separate from the charging station without the ambulatory medicament device establishing communication with the remote system.

Clause 91. The computer-implemented method of clause 90, wherein the transceiver comprises a near-field transceiver or a short-range transceiver.

Clause 92. The computer-implemented method of clause 91, wherein the transceiver comprises a near-field communication transceiver, a Wi-Fi HaLow transceiver, a Zigbee transceiver, a Z-wave transceiver, or a custom short-range transceiver.

Clause 93. The computer-implemented method of clause 90, wherein the charging circuit comprises a wireless charging circuit configured to interface with a wireless charging assembly of the charging station.

Clause 94. The computer-implemented method of clause 90, wherein the charging circuit comprises an inductive charging circuit configured to inductively receive power from the charging station.

Clause 95. The computer-implemented method of clause 90, further comprising registering the ambulatory medicament device with the charging station.

Clause 96. The computer-implemented method of clause 95, wherein registering the ambulatory medicament device with the charging station comprises providing to the charging station a unique identifier associated with the ambulatory medicament device or a subject that receives therapy from the ambulatory medicament device.

Clause 97. The computer-implemented method of clause 95, wherein registering the ambulatory medicament device with the charging station comprises providing, to the charging station, access information for accessing the remote system on behalf of a subject that receives therapy from the ambulatory medicament device.

Clause 98. The computer-implemented method of clause 90, wherein the plurality of data selection criteria comprises: a determination that an alarm has occurred at the ambulatory medicament device; a determination that device data of the ambulatory medicament device indicates occurrence of a malfunction at the ambulatory medicament device; a determination that a time period has elapsed; a determination of a time of a previous transmission of previous ambulatory medicament device data; a determination that a request for the ambulatory medicament device data has been received; a determination that clinical data does not satisfy expected clinical data; a determination that user interface data does not satisfy an expected user interaction profile; and a determination that a backup therapy protocol has been generated or modified.

Clause 99. The computer-implemented method of clause 90, wherein the ambulatory medicament device data comprises one or more of clinical data corresponding to medicament therapy provided by the ambulatory medicament device, device data corresponding to operation of the ambulatory medicament device, or user interface data corresponding to user interaction with the ambulatory medicament device.

Clause 100. The computer-implemented method of clause 90, further comprising: detecting a charge connection to a second charging station; determining that a data connection to the second charging station is not authorized; and initiating, via the charging circuit, charging of the battery over the charge connection to the second charging station without permitting transmission of the ambulatory medicament device data to the second charging station.

Clause 101. The computer-implemented method of clause 90, wherein the ambulatory medicament device further comprises a universal serial bus plug or a universal serial bus port, wherein the transceiver comprises a universal serial bus transceiver in communication with the universal serial bus plug or the universal serial bus port, and wherein the charge connection is established via the universal serial bus plug or the universal serial bus port.

Clause 102. The computer-implemented method of clause 90, wherein transmitting the ambulatory medicament device data to the charging station further comprises transmitting a destination identifier corresponding to the remote system to the charging station.

Clause 103. The computer-implemented method of clause 102, wherein the destination identifier comprises an identification of an account at the remote system, and wherein the account is associated with a subject that receives medicament therapy from the ambulatory medicament device.

Clause 104. The computer-implemented method of clause 90, further comprising encoding the ambulatory medicament device data to obtain encoded data.

Clause 105. The computer-implemented method of clause 104, wherein transmitting the ambulatory medicament device data to the charging station comprises transmitting the encoded data.

Clause 106. The computer-implemented method of clause 104, wherein encoding the ambulatory medicament device data comprises encapsulating the ambulatory medicament device data in one or more transmit packets addressed to the remote system.

Clause 107. The computer-implemented method of clause 104, wherein encoding the ambulatory medicament device data comprises formatting the ambulatory medicament device data for transmission over the data connection.

Clause 108. The computer-implemented method of clause 90, further comprising: accessing account access information associated with an account of a subject at the remote system, wherein the subject receives medicament therapy from the ambulatory medicament device; and transmitting the account access information to the charging station enabling the charging station to access the account of the subject at the remote system.

Clause 109. The computer-implemented method of clause 108, further comprising storing the account access information in a memory of the ambulatory medicament device.

Clause 110. The computer-implemented method of clause 90, further comprising determining that the data connection to the charging station is authorized.

Clause 111. The computer-implemented method of clause 110, further comprising establishing the data connection to the charging station in response to determining that the charging station supports the data connection and that the data connection to the charging station is authorized.

Clause 112. The computer-implemented method of clause 110, wherein determining that the data connection to the charging station is authorized comprises: accessing a unique identifier of the charging station; and determining that the unique identifier of the charging station is registered with the ambulatory medicament device.

Clause 113. The computer-implemented method of clause 90, wherein the data connection comprises a secure connection using asymmetric encryption.

Clause 114. The computer-implemented method of clause 90, wherein at least one of the plurality of data selection criteria is based at least in part on a prior transmission time corresponding to a prior transmission of prior ambulatory medicament device data.

Clause 115. The computer-implemented method of clause 114, wherein the prior transmission of prior ambulatory medicament device data is to the remote system.

Clause 116. The computer-implemented method of clause 114, wherein the prior transmission of prior ambulatory medicament device data is to the charging station or a second charging station.

Clause 117. The computer-implemented method of clause 114, further comprising storing the prior transmission time in a memory of the ambulatory medicament device.

Clause 118. The computer-implemented method of clause 114, further comprising accessing the prior transmission time from the charging station.

Clause 119. The computer-implemented method of clause 114, further comprising transmitting a query request to the charging station to query the remote system for the prior transmission time.

Clause 120. The computer-implemented method of clause 90, wherein at least one of the plurality of data selection criteria is based at least in part on a determination of prior ambulatory medicament device data transmitted to the charging station or to the remote system.

Clause 121. The computer-implemented method of clause 120, wherein the determination of the prior ambulatory medicament device data transmitted to the charging station or to the remote system is determined by accessing a communication record stored in a memory of the ambulatory medicament device.

Clause 122. The computer-implemented method of clause 120, wherein the determination of the prior ambulatory medicament device data transmitted to the charging station or to the remote system is determined by transmitting a query to the charging station to receive an indicator corresponding to at least some of the prior ambulatory medicament device data.

Clause 123. The computer-implemented method of clause 122, wherein the indicator comprises a confirmation of successful transmission of the prior ambulatory medicament device data to the remote system.

Clause 124. An ambulatory medicament device configured to modify or enable modification of a control algorithm of the ambulatory medicament device based on configuration data accessed from a configuration chip, the ambulatory medicament device comprising: a chip reader configured to read data stored on the configuration chip; a memory configured to store specific computer-executable instructions; and a hardware processor in communication with the memory and configured to execute the specific computer-executable instructions to at least: cause the chip reader to emit an electromagnetic pulse; in response to the electromagnetic pulse, receive the configuration data from the configuration chip; decode the configuration data to obtain a value corresponding to a control parameter, wherein the control parameter is used by the control algorithm that generates a dose control signal to cause the ambulatory medicament device to administer a quantity of medicament from a medicament reservoir into a subject; and configure the control parameter based at least in part on the value decoded from the configuration data.

Clause 125. The ambulatory medicament device of clause 124, wherein the configuration chip comprises a radio frequency identification chip or a terahertz frequency identification chip.

Clause 126. The ambulatory medicament device of clause 124, wherein the configuration chip comprises a passive configuration chip or a semi-passive configuration chip.

Clause 127. The ambulatory medicament device of clause 124, wherein the configuration data comprises a configuration code that encodes the value corresponding to the control parameter.

Clause 128. The ambulatory medicament device of clause 124, wherein the configuration data comprises at least one of a plurality of encoded dosing parameters, and wherein the plurality of encoded dosing parameters correspond to a set of dosing parameters comprising: a correction dosing parameter used by the control algorithm to determine a correction bolus of medicament to administer to the subject; a food intake dosing parameter used by the control algorithm to determine a food intake bolus size of medicament to administer to the subject; and a basal dosing parameter used by the control algorithm to determine a basal rate of medicament to administer to the subject.

Clause 129. The ambulatory medicament device of clause 124, wherein configuring the control parameter comprises setting the control parameter to the value decoded from the configuration data.

Clause 130. The ambulatory medicament device of clause 124, wherein the hardware processor is further configured to decode the configuration data by decrypting the configuration data.

Clause 131. The ambulatory medicament device of clause 124, wherein the value comprises an initial value for the control parameter, and wherein the control parameter is adapted over a time period based at least in part on medicament therapy administered over the time period.

Clause 132. The ambulatory medicament device of clause 124, wherein the hardware processor is further configured to verify a checksum of the configuration data prior to decoding the configuration data.

Clause 133. The ambulatory medicament device of clause 124, wherein the hardware processor is further configured to verify that a reference code of the ambulatory medicament device matches a reference code of the configuration data prior to configuring the control parameter of the control algorithm.

Clause 134. The ambulatory medicament device of clause 133, wherein the reference code of the ambulatory medicament device comprises: at least a portion of a serial number, a software version number, or a model number.

Clause 135. The ambulatory medicament device of clause 124, wherein the hardware processor causes the chip reader to emit the electromagnetic pulse in response to a user interaction with a user interface of the ambulatory medicament device.

Clause 136. The ambulatory medicament device of clause 124, wherein the hardware processor causes the chip reader to emit the electromagnetic pulse in response to a proximity detection signal received in response to the configuration chip being within a threshold proximity of the chip reader.

Clause 137. The ambulatory medicament device of clause 124, wherein the configuration data corresponds to a backup therapy protocol.

Clause 138. The ambulatory medicament device of clause 124, wherein the configuration data is associated with the subject or a demographic that includes the subject.

Clause 139. The ambulatory medicament device of clause 124, wherein the chip reader comprises a near-field communication device.

Clause 140. The ambulatory medicament device of clause 124, wherein the configuration chip comprises a near-field communication device incorporated into an electronic device.

Clause 141. The ambulatory medicament device of clause 140, wherein the electronic device comprises a smartphone, a tablet, a smartwatch, smart glasses, a key fob, or portable memory device.

Clause 142. The ambulatory medicament device of clause 140, wherein the electronic device comprises a second ambulatory medicament device.

Clause 143. The ambulatory medicament device of clause 124, wherein the control parameter is associated with a counter-regulatory agent.

Clause 144. The ambulatory medicament device of clause 143, wherein configuring the control parameter triggers administering of a rescue dose of the counter-regulatory agent.

Clause 145. The ambulatory medicament device of clause 124, wherein configuring the control parameter based at least in part on the value decoded from the configuration data modifies execution or enables modification of execution of the control algorithm.

Clause 146. The ambulatory medicament device of clause 124, wherein configuring the control parameter based at least in part on the value decoded from the configuration data enables a previously unavailable feature of the ambulatory medicament device for at least a threshold period of time.

Clause 147. The ambulatory medicament device of clause 124, wherein the configuration data comprises a configuration code having a length of 16 characters or more.

Clause 148. The ambulatory medicament device of clause 124, wherein the configuration data comprises a configuration code having a length of 2 kilobytes or more.

Clause 149. A computer-implemented method of modifying or enabling modification of a control algorithm of an ambulatory medicament device based on configuration data accessed from a configuration chip using a chip reader of the ambulatory medicament device, the computer-implemented method comprising: by a hardware processor of the ambulatory medicament device, causing the chip reader to emit an electromagnetic pulse; in response to the electromagnetic pulse, receiving the configuration data from the configuration chip; decoding the configuration data to obtain a value corresponding to a control parameter, wherein the control parameter is used by the control algorithm that generates a dose control signal to cause the ambulatory medicament device to administer a quantity of medicament from a medicament reservoir into a subject; and configuring the control parameter based at least in part on the value decoded from the configuration data.

Clause 150. The computer-implemented method of clause 149, wherein the configuration chip comprises a radio frequency identification chip or a terahertz frequency identification chip.

Clause 151. The computer-implemented method of clause 149, wherein the configuration chip comprises a passive configuration chip or a semi-passive configuration chip.

Clause 152. The computer-implemented method of clause 149, wherein the configuration data comprises a configuration code that encodes the value corresponding to the control parameter.

Clause 153. The computer-implemented method of clause 149, wherein the configuration data comprises at least one of a plurality of encoded dosing parameters, and wherein the plurality of encoded dosing parameters correspond to a set of dosing parameters comprising: a correction dosing parameter used by the control algorithm to determine a correction bolus of medicament to administer to the subject; a food intake dosing parameter used by the control algorithm to determine a food intake bolus size of medicament to administer to the subject; and a basal dosing parameter used by the control algorithm to determine a basal rate of medicament to administer to the subject.

Clause 154. The computer-implemented method of clause 149, wherein configuring the control parameter comprises setting the control parameter to the value decoded from the configuration data.

Clause 155. The computer-implemented method of clause 149, wherein the configuration data received from the configuration chip is encrypted, and wherein the computer-implemented method further comprises decoding the configuration data by decrypting the configuration data.

Clause 156. The computer-implemented method of clause 149, wherein the value comprises an initial value for the control parameter, and wherein the computer-implemented method further comprises adapting the control parameter over a time period based at least in part on medicament therapy administered to a subject over the time period.

Clause 157. The computer-implemented method of clause 149, further comprising verifying a checksum of the configuration data prior to decoding the configuration data.

Clause 158. The computer-implemented method of clause 149, further comprising verifying that a reference code of the ambulatory medicament device matches a reference code of the configuration data prior to configuring the control parameter of the control algorithm.

Clause 159. The computer-implemented method of clause 158, wherein the reference code of the ambulatory medicament device comprises: at least a portion of a serial number, a software version number, or a model number.

Clause 160. The computer-implemented method of clause 149, further comprising causing the chip reader to emit the electromagnetic pulse in response to a user interaction with a user interface of the ambulatory medicament device.

Clause 161. The computer-implemented method of clause 149, further comprising causing the chip reader to emit the electromagnetic pulse in response to a proximity detection signal received in response to the configuration chip being within a threshold proximity of the chip reader.

Clause 162. The computer-implemented method of clause 149, wherein the configuration data corresponds to a backup therapy protocol.

Clause 163. The computer-implemented method of clause 149, wherein the configuration data is associated with the subject or a demographic that includes the subject.

Clause 164. The computer-implemented method of clause 149, wherein the chip reader comprises a near-field communication device.

Clause 165. The computer-implemented method of clause 149, wherein the configuration chip comprises a near-field communication device incorporated into an electronic device.

Clause 166. The computer-implemented method of clause 165, wherein the electronic device comprises a smartphone, a tablet, a smartwatch, smart glasses, a key fob, or portable memory device.

Clause 167. The computer-implemented method of clause 165, wherein the electronic device comprises a second ambulatory medicament device.

Clause 168. The computer-implemented method of clause 149, wherein the control parameter is associated with a counter-regulatory agent.

Clause 169. The computer-implemented method of clause 168, wherein configuring the control parameter triggers administering of a rescue dose of the counter-regulatory agent.

Clause 170. The computer-implemented method of clause 149, wherein configuring the control parameter based at least in part on the value decoded from the configuration data modifies execution or enables modification of execution of the control algorithm.

Clause 171. The computer-implemented method of clause 149, wherein configuring the control parameter based at least in part on the value decoded from the configuration data enables a previously unavailable feature of the ambulatory medicament device for at least a threshold period of time.

Clause 172. The computer-implemented method of clause 149, wherein the configuration data comprises a configuration code having a length of 16 characters or more.

Clause 173. The computer-implemented method of clause 149, wherein the configuration data comprises a configuration code having a length of 2 kilobytes or more.

In other embodiments, a system or systems may operate according to one or more of the methods and/or computer-readable media recited in the preceding paragraphs. In yet other embodiments, a method or methods may operate according to one or more of the systems and/or computer-readable media recited in the preceding paragraphs. In yet more embodiments, a computer-readable medium or media, excluding transitory propagating signals, may cause one or more computing devices having one or more processors and non-transitory computer-readable memory to operate according to one or more of the systems and/or methods recited in the preceding paragraphs.

### Terminology

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware. Further, the computing system may include, be implemented as part of, or communicate with an automated blood glucose system, an ambulatory medicament system, or an ambulatory medical device.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like A processor can include electrical circuitry configured to process computer-executable instructions. In another embodiment, a processor includes an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. A computing environment can include any type of computer system, including, but not limited to, a computer system based on a microprocessor, a mainframe computer, a digital signal processor, a portable computing device, a device controller, or a computational engine within an appliance, to name a few.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, etc., may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

It should be emphasized that many variations and modifications may be made to the above-described embodiments, the elements of which are to be understood as being among other acceptable examples. All such modifications and variations are intended to be included herein within the scope of this disclosure.

1-53. (canceled)

54. A computer-implemented method of transmitting ambulatory medicament device data to a charging station by an ambulatory medicament device comprising:
a charging circuit configured to receive power from the charging station and to charge a battery of the ambulatory medicament device, and a transceiver configured to at least transmit the ambulatory medicament device data to the charging station, the computer-implemented method further comprising: by a hardware processor of the ambulatory medicament device, detecting a charge connection to the charging station, initiating, via the charging circuit, charging of the battery over the charge connection; determining whether the charging station supports a data connection to the ambulatory medicament device; and in response to determining that the charging station supports the data connection: establishing the data connection to the charging station; automatically selecting, without user interaction with a user interface, ambulatory medicament device data using one or more of a plurality of data selection criteria; preparing the ambulatory medicament device data for transmission to the charging station via the data connection; and automatically transmitting, without user interaction with the user interface, the ambulatory medicament device data of the charging station enabling communication of the ambulatory medicament device data by the charging station to a remote system separate from the charging station without the ambulatory medicament device establishing communication with the remote system

55. The computer-implemented method of claim 54, wherein the transceiver comprises at least one of a near-field transceiver and a short-range transceiver.

56. The computer-implemented method of claim 55, wherein the transceiver comprises a near-field communication transceiver, a Wi-Fi HaLow transceiver, a Zigbee transceiver, a Z-wave transceiver, or a custom short-range transceiver.

57. The computer-implemented method of claim 54, wherein the charging circuit comprises a wireless charging circuit configured to interface with a wireless charging assembly of the charging station.

58. The computer-implemented method of claim 54, wherein the charging circuit comprises an inductive charging circuit configured to inductively receive power from the charging station.

59. The computer-implemented method of claim 54, further comprising registering the ambulatory medicament device with the charging station.

60. The computer-implemented method of claim 59, wherein registering the ambulatory medicament device with the charging station comprises providing to the charging station a unique identifier associated with the ambulatory medicament device or a subject that receives therapy from the ambulatory medicament device.

61. The computer-implemented method of claim 60, wherein registering the ambulatory medicament device with the charging station comprises providing, to the charging station, access information for accessing the remote system on behalf of a subject that receives therapy from the ambulatory medicament device.

62. The computer-implemented method of claim 54, wherein the plurality of data selection criteria comprises: a determination that an alarm has occurred at the ambulatory medicament device; a determination that device data of the ambulatory medicament device indicates occurrence of a malfunction at the ambulatory medicament device; a determination that a time period has elapsed; a determination of a time of a previous transmission of previous ambulatory medicament device data; a determination that a request for the ambulatory medicament device data has been received, a determination that clinical data does not satisfy expected clinical data; a determination that user interface data does not satisfy an expected user interaction profile; and a determination that a backup therapy protocol has been generated or modified.

63. The computer-implemented method of claim 54, wherein the ambulatory medicament device data comprises one or more of clinical data corresponding to medicament therapy provided by the ambulatory medicament device, device data corresponding to operation of the ambulatory medicament device, or user interface data corresponding to user interaction with the ambulatory medicament device.

64. The computer-implemented method of claim 54, further comprising: detecting a charge connection to a second charging station; determining that a data connection to the second charging station is not authorized, and initiating, via the charging circuit, charging of the battery over the charge connection to the second charging station without permitting transmission of the ambulatory medicament device data to the second charging station.

65. The computer-implemented method of claim 54, wherein transmitting the ambulatory medicament device data to the charging station further comprises transmitting a destination identifier corresponding to the remote system to the charging station.

66. The computer-implemented method of claim 54, wherein at least one of the plurality of data selection criteria is based at least in part on a determination of prior ambulatory medicament device data transmitted to the charging station or to the remote system.

67. The computer-implemented method of claim 54, wherein the determination of the prior ambulatory medicament device data transmitted to the charging station or to the remote system is determined by accessing a communication record stored in a memory of the ambulatory medicament device, further comprising at least:
Pump being on station;
Establishment of at least wireless and wiFi connection; and
Both local network interfaced connection and WAN to cloud.

68. The computer-implemented method of claim 67, wherein the determination of the prior ambulatory medicament device data transmitted to the charging station or to the remote system is determined by transmitting a query to the charging station to receive an indicator corresponding to at least some of the prior ambulatory medicament device data.

69. The computer-implemented method of claim 68, wherein the indicator comprises a confirmation of successful transmission of the prior ambulatory medicament device data to the remote system.

70. A computer-implemented method of transmitting ambulatory medicament device data to a charging station by an ambulatory medicament device comprising:
a charging circuit configured to receive power from the charging station and to charge a battery of the ambulatory medicament device, and a transceiver configured to at least transmit the ambulatory medicament device data to the charging station, the computer-implemented method further comprising: by a hardware processor of the ambulatory medicament device, detecting a charge connection to the charging station, initiating, via the charging circuit, charging of the battery over the charge connection; automatically selecting, without user interaction with a user interface, ambulatory medicament device data using one or more of a plurality of data selection criteria; preparing the ambulatory medicament device data for transmission to the charging station via the data connection; and automatically transmitting, without user interaction with the user interface, the ambulatory medicament device data of the charging station enabling communication of the ambulatory medicament device data by the charging station to a remote system separate from the charging station without the ambulatory medicament device establishing communication with the remote system;
whereby when pump is on station a plurality of communication channels and protocols are connected charge review, status and updating occurs with Pump being on station;
Establishment of at least wireless and wiFi connection; and
Both local network interfaced connection and WAN to cloud.

71. The method of claim 70, further comprising the second transceiver being dedicated to device data.
